(19) United States
(12) Patent Application Publication
(10) Pub. No.: US 2026/0000735 A1
(43) Pub. Date: Jan. 1, 2026

(54) ENHANCED DIFFERENTIATION OF BETA CELLS

(71) Applicant: Vertex Pharmaceuticals Incorporated, Boston, MA (US)

(72) Inventor: Bryce Carey, Cambridge, MA (US)

(73) Assignee: Vertex Pharmaceuticals Incorporated, Boston, MA (US)

(21) Appl. No.: 19/300,446

(22) Filed: Aug. 14, 2025

Related U.S. Application Data
(63) Continuation of application No. 17/560,680, filed on Dec. 23, 2021.
Application No. 17/560,680 is a continuation of application No. PCT/US2020/039487, filed on Jun. 25, 2020.
(60) Provisional application No. 62/866,100, filed on Jun. 25, 2019.

(51) Int. Cl. A61K 38/28; A61K 31/192; A61K 35/37; C12N 5/071

(52) U.S. Cl. CPC A61K 38/28; A61K 31/192; A61K 35/37; C12N 5/0676; C12N 2500/22; C12N 2500/33; C12N 2500/36; C12N 2506/03

(57) ABSTRACT

Provided herein are methods of manufacturing β cells in vitro. Also provided herein are methods of treating a disease in a subject comprising administering the β cells manufactured in vitro to the subject. Also provided herein are methods of differentiating stem cells into β cells.

### CROSS-REFERENCE TO RELATED APPLICATIONS

This is a continuation of U.S. patent application Ser. No. 17/560,680, filed on Dec. 23, 2021, which is a continuation of international Patent Application No. PCT/US2020/039487, filed Jun. 25, 2020, which claims the benefit of U.S. Provisional Patent Application No. 62/866,100, fled on Jun. 25, 2019, each of which is hereby incorporated by reference in its entirety.

### BACKGROUND

Transplantation of pancreas or pancreatic islets has been used for treating diabetes, such as type I diabetes. Pancreatic islet transplantation does not need major surgery and the function of the islet grafts can be maintained for years in a recipient. However, a shortage of pancreatic islets donors prevents this therapy from being effectively implemented. Artificial pancreas or pancreatic islets provide an alternative source of transplantable islets.

### SUMMARY

In some embodiments, the disclosure provides for a composition comprising dissociated cells. In some embodiments, the composition does not comprise any cell clusters. In some embodiments, the composition does not comprise any insulin-positive cell clusters. In some embodiments, the composition does not comprise any cell clusters comprising more than 5, 10, 20, 30, 50, 100, 200, 300, 400, 500, 600, 700, 800, 900, or 1000 cells. In some embodiments, the composition does not comprise any cell clusters comprising more than 50 cells. In some embodiments, the composition does not comprise any cell clusters comprising more than 100 cells. In some embodiments, the composition does not comprise any cell clusters comprising more than 500 cells. In some embodiments, the disclosure provides for a composition comprising a plurality of dissociated insulin-positive endocrine progenitor cells. In some embodiments, the dissociated cells are Ngn3-positive. In some embodiments, the dissociated cells are PDX.1 positive. In some embodiments, the dissociated cells are NKX6.1 positive. In some embodiments, the disclosure provides for a composition comprising dissociated cells (e.g., dissociated insulin-positive endocrine progenitor cells) and a BMP signaling pathway inhibitor. In some embodiments, the BMP signaling pathway inhibitor is LDN193189 or a derivative thereof. In some embodiments, the disclosure provides for a composition comprising a plurality of dissociated cells (e.g., dissociated insulin-positive endocrine progenitor cells) and a ROCK inhibitor. In some embodiments, the ROCK inhibitor is thiazovivin, Y-27632, Fasudil/HA1077, or 14-1152, or derivatives thereof. In some embodiments, the disclosure provides for a composition comprising a plurality of dissociated cells (e.g., dissociated insulin-positive endocrine progenitor cells) and a histone methyltransferase inhibitor. In some embodiments, the histone methyltransferase inhibitor is 3-Deazaneplanocin A hydrochloride, or a derivative thereof. In some embodiments, the disclosure provides for a composition comprising a plurality of dissociated cells (e.g., dissociated insulin-positive endocrine progenitor cells) and zinc. In some embodiments, the zinc is in the form of ZnSO₄. In some embodiments, the disclosure provides for a composition comprising a plurality of dissociated cells (e.g., dissociated insulin-positive endocrine progenitor cells) and a monoglyceride lipase (MGLL) inhibitor. In some embodiments, the MGLL inhibitor is JJKK048, KML29, NF1819, JW642, JZL184, JZL195, JZP361, pristimerin, or URB602, or a derivative of any of the foregoing. In some embodiments, the disclosure provides for a composition comprising a plurality of dissociated cells (e.g., dissociated insulin-positive endocrine progenitor cells) and a lipid. In some embodiments, the lipid is a saturated fatty acid. In some embodiments, the saturated fatty acid is palmitate. In some embodiments, the lipid is a unsaturated fatty acid. In some embodiments, the unsaturated fatty acid is oleic acid, linoleic acid, or palmitoleic acid. In some embodiments, the disclosure provides for a composition comprising a plurality of dissociated cells (e.g., dissociated insulin-positive endocrine progenitor cells) and glutamate. In some embodiments, the disclosure provides for a composition comprising a plurality of dissociated cells (e.g., dissociated insulin-positive endocrine progenitor cells) and acetate. In some embodiments, the disclosure provides for a composition comprising a plurality of dissociated cells (e.g., dissociated insulin-positive endocrine progenitor cells) and β-hydroxybutarate. In some embodiments, the disclosure provides for a composition comprising a plurality of dissociated cells (e.g., dissociated insulin-positive endocrine progenitor cells) and L-carnitine. In some embodiments, the disclosure provides for a composition comprising a plurality of dissociated cells (e.g., dissociated insulin-positive endocrine progenitor cells) and taurine. In some embodiments, the disclosure provides for a composition comprising a plurality of dissociated cells (e.g., dissociated insulin-positive endocrine progenitor cells) and formate. In some embodiments, the disclosure provides for a composition comprising a plurality of dissociated cells (e.g., dissociated insulin-positive endocrine progenitor cells) and biotin. In some embodiments, the composition further comprises a serum albumin protein. In some embodiments, the serum albumin protein is a human serum albumin protein. In some embodiments, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some embodiments, less than 90%, less than 85%, les thant 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some embodiments, the composition comprises a TGF-β pathway inhibitor. In some embodiments, the TGF-β pathway inhibitor is Alk5i (SB505124), or a derivative thereof. In some embodiments, the composition comprises a thyroid hormone signaling pathway activator. In some embodiments, the thyroid hormone signaling pathway activator is GC-1 or T3, or a derivative thereof. In some embodiments, the composition comprises a protein kinase inhibitor. In some embodiments, the protein kinase inhibitor is staurosporine. In some embodiments, the composition comprises vitamin C. In some embodiments, the composition comprises insulin. In particular embodiments, the composition is in vitro. In some embodiments, the composition does not comprise a γ secretase inhibitor (e.g., XXI). In some embodiments, the dissociated insulin-positive endocrine progenitor cells were previously frozen.

In some embodiments, the disclosure provides for a method comprising the step of contacting a plurality of dissociated insulin-positive endocrine progenitor cells with a BMP signaling pathway inhibitor. In some embodiments, the BMP signaling pathway inhibitor is LDN193189 or a derivative thereof. In some embodiments, the disclosure provides for a method comprising the step of contacting a plurality of dissociated insulin-positive endocrine progenitor cells with a ROCK inhibitor. In some embodiments, the ROCK inhibitor is thiazovivin, Y-27632, Fasudil/HA1077, or 14-1152, or derivatives thereof. In some embodiments, the disclosure provides for a method comprising the step of contacting a plurality of dissociated insulin-positive endocrine progenitor cells with a histone methyltransferase inhibitor. In some embodiments, the histone methyltransferase inhibitor is 3-Deazaneplanocin A hydrochloride, or a derivative thereof. In some embodiments, the disclosure provides for a method comprising the step of contacting a plurality of dissociated insulin-positive endocrine progenitor cells with zinc. In some embodiments, the zinc is in the form of ZnSO₄. In some embodiments, the disclosure provides for a method comprising the step of contacting a plurality of dissociated insulin-positive endocrine progenitor cells with a monoglyceride lipase (MGLL) inhibitor. In some embodiments, the MGLL inhibitor is JJKK048, KML29, NF1819, JW642, JZL184, JZL195, JZP361, pristimerin, or URB602, or a derivative of any of the foregoing. In some embodiments, the disclosure provides for a method comprising the step of contacting a plurality of dissociated insulin-positive endocrine progenitor cells with a lipid. In some embodiments, the lipid is a saturated fatty acid. In some embodiments, the saturated fatty acid is palmitate. In some embodiments, the lipid is an unsaturated fatty acid. In some embodiments, the unsaturated fatty acid is oleic acid, linoleic acid, or palmitoleic acid. In some embodiments, the disclosure provides for a method comprising the step of contacting a plurality of dissociated insulin-positive endocrine progenitor cells with glutamate. In some embodiments, the disclosure provides for a method comprising the step of contacting a plurality of dissociated insulin-positive endocrine progenitor cells with acetate. In some embodiments, the disclosure provides for a method comprising the step of contacting a plurality of dissociated insulin-positive endocrine progenitor cells with β-hydroxybutarate. In some embodiments, the disclosure provides for a method comprising the step of contacting a plurality of dissociated insulin-positive endocrine progenitor cells with L-carnitine. In some embodiments, the disclosure provides for a method comprising the step of contacting a plurality of dissociated insulin-positive endocrine progenitor cells with taurine. In some embodiments, the disclosure provides for a method comprising the step of contacting a plurality of dissociated insulin-positive endocrine progenitor cells with formate. In some embodiments, the disclosure provides for a method comprising the step of contacting a plurality of dissociated insulin-positive endocrine progenitor cells with biotin. In some embodiments, the method comprises contacting the plurality of dissociated insulin-positive endocrine progenitor cells with a serum albumin protein. In some embodiments, the serum albumin protein is a human serum albumin protein. In some embodiments, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some embodiments, less than 90%, less than 85%, less than 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some embodiments, the method comprises contacting the plurality of dissociated insulin-positive endocrine progenitor cells with a TGF-β pathway inhibitor. In some embodiments, the TGF-β pathway inhibitor is Alk5i (SB505124), or a derivative thereof. In some embodiments, the method comprises contacting the plurality of dissociated insulin-positive endocrine progenitor cells with a thyroid hormone signaling pathway activator. In some embodiments, the thyroid hormone signaling pathway activator is GC-1 or T3, or a derivative thereof. In some embodiments, the method comprises contacting the plurality of dissociated insulin-positive endocrine progenitor cells with a protein kinase inhibitor. In some embodiments, the protein kinase inhibitor is staurosporine. In some embodiments, the method comprises contacting the plurality of dissociated insulin-positive endocrine progenitor cells with vitamin C. In some embodiments, the method comprises contacting the plurality of dissociated insulin-positive endocrine progenitor cells with insulin. In some embodiments, the method does not comprise the step of contacting the plurality of dissociated insulin-positive endocrine cells with a γ secretase inhibitor (e.g., XXI). In some embodiments, the dissociated insulin-positive endocrine progenitor cells were previously frozen. In some embodiments, the method is performed over the course of 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2- 6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days. In some embodiments, the method results in the reaggregation of the dissociated cells into a plurality of cell clusters. In some embodiments, at least about 40%, 50%, 60%, 70%, 80%, or 90% of the plurality of cell clusters have a diameter from about 50 μm to about 250 μm, from about 75 μm to about 250 μm, or from about 100 μm to about 200 μm. In some embodiments, at least about 40%, 50%, 60%, 65%, 70%, 75%, 80%, 85%, 95%, or 99% of the cells of the plurality of cell clusters of the second cell population are viable. In some embodiments, the method results in the reaggregation of the dissociated cells into at least 2, 3, 4, 5, 10, 50, 100, 1000, 10000, 100000, or 1000000 cell clusters.

In some embodiments, the disclosure provides for a composition comprising a plurality of cell clusters. In some embodiments, the disclosure provides for a composition comprising a plurality of cell clusters; wherein the cell clusters comprise insulin-positive cells; wherein at least 30%, at least 35%, at least 40%, at least 45%, at least 50%, at least 55%, at least 60%, or at least 65% of the cells in the composition are viable following 11 days in culture in vitro. In some embodiments, the disclosure provides for a composition comprising a plurality of cell clusters; wherein the cell clusters comprise insulin-positive cells; wherein at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter. In some embodiments, the disclosure provides for a composition comprising a plurality of cell clusters; wherein the cell clusters comprise insulin-positive cells; wherein at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, the cell clusters comprise C-peptide positive cells. In some embodiments, the cell clusters comprise somatostatin positive cells. In some embodiments, the cell clusters comprise glucagon positive cells. In some embodiments, at least 30%, at least 35%, at least 40%, at least 45%, at least 50%, at least 55%, at least 60%, or at least 65% of the cells in the composition are viable following 11 days in culture in vitro. In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter. In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, at least 2, 3, 4, 5, 10, 50, 100, 1000, 10000, 100000, or 1000000 cell clusters. In some embodiments, the composition is prepared in accordance with any of the methods disclosed herein. In some embodiments, the disclosure provides for a device comprising any of the cell compositions disclosed herein. In some embodiments, the disclosure provides for a method of treating a subject with a disease characterized by high blood sugar levels over a prolonged period of time (e.g., diabetes), the method comprising administering any of the compositions disclosed herein or any of the devices disclosed herein to the subject.

In some embodiments, the disclosure provides for a composition comprising a plurality of cell clusters; wherein the cell clusters comprise insulin-positive cells; wherein at least 30%, at least 35%, at least 40%, at least 45%, at least 50%, at least 55%, at least 60%, or at least 65% of the cells in the composition are viable following 11 days in culture in vitro. In some embodiments, the disclosure provides for a composition comprising a plurality of cell clusters; wherein the cell clusters comprise insulin-positive cells; wherein at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter. In some embodiments, the disclosure provides for a composition comprising a plurality of cell clusters; wherein the cell clusters comprise insulin-positive cells; wherein at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, the cell clusters comprise C-peptide positive cells. In some embodiments, the cell clusters comprise somatostatin positive cells. In some embodiments, the cell clusters comprise glucagon positive cells. In some embodiments, at least 30%, at least 35%, at least 40%, at least 45%, at least 50%, at least 55%, at least 60%, or at least 65% of the cells in the composition are viable following 11 days in culture in vitro. In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter. In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, at least 2, 3, 4, 5, 10, 50, 100, 1000, 10000, 100000, or 1000000 cell clusters. In some embodiments, the composition is prepared in accordance with any of the methods disclosed herein. In some embodiments, the disclosure provides for a device comprising any of the cell compositions disclosed herein. In some embodiments, the disclosure provides for a method of treating a subject with a disease characterized by high blood sugar levels over a prolonged period of time (e.g., diabetes), the method comprising administering any of the compositions disclosed herein or any of the devices disclosed herein to the subject.

Provided herein are methods comprising: (a) obtaining a first population of cells comprising a plurality of cell clusters comprising insulin-positive cells; (b) dissociating at least a portion of the plurality of cell clusters in the first population of cells in vitro; (c) contacting the first population of cells comprising at least a portion of the dissociated cell clusters with a first composition in vitro, wherein the first composition comprises at least one of the following agents: a monoglyceride lipase (MGLL) inhibitor, a bone morphogenic protein (BMP) type 1 receptor inhibitor, a Rho-associated coiled-coil containing protein kinase (ROCK) inhibitor, a histone methyltransferase inhibitor, or a protein kinase inhibitor, to obtain a second population of cells comprising a plurality of cells clusters comprising a plurality of insulin-positive cells; and (d) contacting the second population of insulin-positive cells in vitro with a second composition, wherein the second composition is different from the first composition, thereby differentiating at least a portion of said second population of insulin-positive cells into a third population of cells comprising a plurality of β cells, wherein the third population of cells comprises a higher percentage of viable β cells as compared to a corresponding population of β cells comprising β cells derived from the first population of cells which is not contacted with the first composition.

Provided herein are methods comprising: (a) obtaining a first population of cells comprising a plurality of cell clusters comprising insulin-positive cells; (b) dissociating at least a portion of the plurality of cell clusters in the first population of cells in vitro; (c) contacting the first population of cells comprising at least a portion of the dissociated cell clusters with a first composition in vitro, wherein the first composition comprises a transforming growth factor β (TGF-β) signaling pathway inhibitor, a thyroid hormone signaling pathway activator, or both, and at least one of the following agents: a monoglyceride lipase (MGLL) inhibitor, a bone morphogenic protein (BMP) type 1 receptor inhibitor, a Rho-associated coiled-coil containing protein kinase (ROCK) inhibitor, a histone methyltransferase inhibitor, or a protein kinase inhibitor, to obtain a second population of cells comprising a plurality cell clusters comprising a plurality of insulin-positive endocrine cells; and (d) contacting the second population of insulin-positive cells in vitro with a second composition, wherein the second composition is different from the first composition, thereby differentiating at least a portion of said second population of insulin-positive cells into a third population of cells comprising a plurality of β cells, wherein the third population of cells comprises a higher percentage of viable β cells as compared to a corresponding population of β cells comprising β cells derived from the first population of cells which is not contacted with the first composition.

Provided herein are methods comprising: (a) obtaining a first population of cells comprising a plurality of cell clusters comprising insulin-positive cells; (b) dissociating at least a portion of the plurality of cell clusters in the first population of cells in vitro; (c) contacting the first population of cells comprising at least a portion of the dissociated cell clusters with a first composition in vitro, wherein the first composition comprises a monoglyceride lipase (MGLL) inhibitor, to obtain a second population of cells comprising a plurality of cells clusters comprising a plurality of insulin-positive endocrine cells; and (d) contacting the second population of insulin-positive cells in vitro with a second composition, wherein the second composition is different from the first composition, thereby differentiating at least a portion of said second population of insulin-positive cells into a third population of cells comprising a plurality of β cells, wherein the third population of cells comprises a higher percentage of viable β cells as compared to a corresponding population of β cells comprising β cells derived from the first population of cells which is not contacted with the first composition.

In some embodiments, the methods further comprise freezing at least a portion of the first population of cells comprising at least a portion of the dissociated cell clusters; thawing at least a portion of the frozen first population of cells; and contacting at least a portion of the first population of thawed cells in vitro with said first composition.

In some embodiments, at least a portion of the plurality of cell clusters of the second cell population have a diameter from about 50 μm to about 250 μm, from about 75 μm to about 250 μm, or from about 100 μm to about 200 μm. In some embodiments, at least about 40%, 50%, 60%, 70%, 80%, or 90% of the plurality of cell clusters of the second cell population have a diameter from about 50 μm to about 250 μm, from about 75 μm to about 250 μm, or from about 100 μm to about 200 μm. In some embodiments, at least about 40%, 50%, 60%, 65%, 70%, 75%, 80%, 85%, 95%, or 99% of the cells of the second cell population are viable. In some embodiments, at least about 40%, 50%, 60%, 65%, 70%, 75%, 80%, 85%, 95%, or 99% of the cells of the plurality of cell clusters of the second cell population are viable.

In some embodiments, the second population of cells comprises at least 2, 3, 4, 5, 10, 50, 100, 1000, 10000, 100000, or 1000000 cell clusters.

In some embodiments, the second population of cells comprises a higher percentage of said insulin-positive endocrine cells as compared to a corresponding population of cells comprising insulin-positive endocrine cells which is not contacted with the first composition. In some embodiments, the second population of cells comprises at least about 10%, 20%, 30%, 40%, 50%, 60%, 70%, 80%, or 90% more viable insulin-positive endocrine cells as compared to a corresponding population of cells comprising insulin-positive endocrine cells which is not contacted with the first composition. In some embodiments, the second population of cells comprises at least about 10%, 20%, 30%, 40%, 50%, 60%, 70%, 80%, or 90% more viable insulin-positive endocrine cells after about 1, 2, 3, 4, 5, 6, 7, 8, 9, or 10 days of contacting the first cell population with the first composition as compared to a corresponding population of cells comprising insulin-positive endocrine cells which is not contacted with the first composition. In some embodiments, the second population of cells comprises at least about 10%, 20%, 30%, 40%, 50%, 60%, 70%, 80%, or 90% more viable insulin-positive endocrine cells after from about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days of contacting the first cell population with the first composition as compared to a corresponding population of cells comprising insulin-positive endocrine cells which is not contacted with the first composition.

In some embodiments, at least a portion of the plurality of β cells forms a plurality of cell clusters. In some embodiments, at least portion of the plurality of cell clusters of the third cell population have a diameter from about 50 μm to about 250 μm, from about 50 μm to about 150 μm, from about 50 μm to about 100 μm, from about 75 μm to about 250 μm, from about 75 μm to about 150 μm, from about 75 μm to about 125 μm, from about 75 μm to about 100 μm, or from about 100 μm to about 200 μm.

In some embodiments, at least about 40%, 50%, 60%, 70%, 80%, or 90% of the plurality of cell clusters of the third cell population have a diameter from about 50 μm to about 250 μm, from about 50 μm to about 150 μm, from about 50 μm to about 100 μm, from about 75 μm to about 250 μm, from about 75 μm to about 150 μm, from about 75 μm to about 125 μm, from about 75 μm to about 100 μm, or from about 100 μm to about 200 μm, in the absence of a selection step. In some embodiments, at least about 40%, 50%, 60%, 70%, 75%, 80%, 90%, or 95% of the cell clusters have a diameter from about 50-150 μm, 75-12 μm, 80-120 μm, or 90-110 μm, in the absence of a selection step. In some embodiments, at least about 50%, 60%, 70%, 75%, 80%, 90%, or 95% of the cell clusters have a diameter of about 100 microns, in the absence of a selection step. In some embodiments, at least about 40%, 50%, 60%, 65%, 70%, 75%, 80%, 85%, 95%, or 99% of the cells of the third cell population are viable, in the absence of a selection step. In some embodiments, at least about 40%, 50%, 60%, 65%, 70%, 75%, 80%, 85%, 95%, or 99% of the cells of the plurality of cell clusters of the third cell population are viable, in the absence of a selection step.

In some embodiments, the third population of cells comprises at least 2, 3, 4, 5, 10, 50, 100, 1000, 10000, 100000, or 1000000 cell clusters. In some embodiments, the third population of cells comprises at least about 10%, 20%, 30%, 40%, 50%, 60%, 70%, 80%, or 90% more viable β cells as compared to a corresponding population of cells comprising β cells derived from the first population of cells which is not contacted with the first composition. In some embodiments, the third population of cells comprises at least about 10%, 20%, 30%, 40%, 50%, 60%, 70%, 80%, or 90% more viable β cells after about 1, 2, 3, 4, 5, 6, 7, 8, 9, or 10 days of contacting the first cell population with the first composition as compared to a corresponding population of cells comprising β cells derived from the first population of cells which is not contacted with the first composition. In some embodiments, the second population of cells comprises at least about 10%, 20%, 30%, 40%, 50%, 60%, 70%, 80%, or 90% more viable β cells after from about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4- 6 days, or 4-5 days of contacting the first cell population with the first composition as compared to a corresponding population of cells comprising β cells derived from the first population of cells which is not contacted with the first composition.

In some embodiments, at least a portion of the plurality of β cells of the third cell population display glucose stimulated insulin secretion (GSIS) in response to a glucose challenge in vitro. In some embodiments, at least a portion of the plurality of β cells of the third cell population express insulin.

In some embodiments, the first composition compromises two, three, four, or five of the agents. In some embodiments, the first composition compromises three, four, five, six, or seven of the agents. In some embodiments, the contacting with the first composition comprises contacting the first population of cells with the first composition for about 1 day, 2 days, 3 days, 4 days, 5 days, 6 days, 7 days, or 8 or more days. In some embodiments, the contacting with the first composition comprises contacting the first population of cells with the first composition for about 4 days. In some embodiments, the contacting with the first composition comprises contacting the first population of cells with the first composition for about 6 hours, 10 hours, 12 hours, 24 hours, 30 hours, 36 hours, 40 hours, 48 hours, 56 hours, 72 hours, or more hours. In some embodiments, the contacting with the first composition comprises contacting the first population of cells with the first composition for from about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2- 5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, 4-5 days, 5-10 days, 5-9 days, 5-8 days, 5-7 days, 5-6 days, 6- 10 days, 6-9 days, 6-8 days, 6-7 days, 7-10 days, 7-8 days, 8-10 days, 8-9, days, or 9-10 days. In some embodiments, the contacting with the first composition comprises contacting the first population of cells with the first composition for about 6-96 hours, 6-72 hours, 6-48 hours, 6-24 hours, 6-12 hours, 12-96 hours, 12-72 hours, 12-48 hours, 12-24 hours, 24-96 hours, 24-72 hours, 24-45 hours, 48-96 hours, or 48-72 hours. In some embodiments, the contacting with the first composition comprises contacting the first population of cells with the first composition for about 72 hours.

In some embodiments, the first composition further comprises a transforming growth factor 3 (TGF-β) signaling pathway inhibitor, a thyroid hormone signaling pathway activator, or both. In some embodiments, the first composition comprises a MGLL inhibitor. In some embodiments, the first composition comprises a TGF-β signaling pathway inhibitor. In some embodiments, the first composition comprises a thyroid hormone signaling pathway activator. In some embodiments, the first composition comprises a bone morphogenic protein (BMP) type 1 receptor inhibitor. In some embodiments, the first composition comprises a Rho-associated coiled-coil containing protein kinase (ROCK) inhibitor. In some embodiments, the first composition comprises a histone methyltransferase inhibitor. In some embodiments, the first composition comprises a protein kinase inhibitor.

In some embodiments, the first composition comprises a TGF-β signaling pathway inhibitor, a thyroid hormone signaling pathway activator, a bone morphogenic protein (BMP) type 1 receptor inhibitor, a Rho-associated coiled-coil containing protein kinase (ROCK) inhibitor, a histone methyltransferase inhibitor, and a protein kinase inhibitor.

In some embodiments, the first composition comprises a MGLL inhibitor, a TGF-β signaling pathway inhibitor, a thyroid hormone signaling pathway activator, a bone morphogenic protein (BMP) type 1 receptor inhibitor, a Rho-associated coiled-coil containing protein kinase (ROCK) inhibitor, a histone methyltransferase inhibitor, and a protein kinase inhibitor.

In some embodiments, the TGF-β signaling pathway inhibitor is Alk5i (SB505124).

In some embodiments, the thyroid hormone signaling pathway activator is a T3 or analog or a derivative thereof.

In some embodiments, the thyroid hormone signaling pathway activator is a TRβ selective agonist-GC-1.

In some embodiments, the thyroid hormone signaling pathway activator is 3,5-dimethyl-4-[(4′-hydroxy-3′-isopropylbenzyl)-phenoxy]acetic acid.

In some embodiments, the bone morphogenic protein (BMP) type 1 receptor inhibitor is LDN193189 or a derivative thereof.

In some embodiments, the Rho-associated coiled-coil containing protein kinase (ROCK) inhibitor is thiazovivin.

In some embodiments, the histone methyltransferase inhibitor is 3-deazaneplanocin A.

In some embodiments, the protein kinase inhibitor is staurosporine (SSP)).

In some embodiments, the first composition does not comprise a γ secretase inhibitor (e.g., XXI), zinc sulfate, or both.

In some embodiments, the first composition further comprises a lipid. In some embodiments, the lipid is a saturated fatty acid. In some embodiments, the saturated fatty acid is palmitate. In some embodiments, the lipid is a unsaturated fatty acid. In some embodiments, the non-saturated fatty acid is oleic acid, linoleic acid, or palmitoleic acid.

In some embodiments, the first composition comprises human serum albumin (HSA). In some embodiments, the first composition comprises from about 0.01-5%, 0.01-4%, 0.01-3%, 0.01-2%, 0.01-1%, 0.01-0.5%, 0.01-0.06%, or 0.01-0.05% HSA. In some embodiments, the first composition comprises more than about 0.01%, 0.02%, 0.03%, 0.04%, 0.05%, 0.06%, 0.07%, 0.08%, 0.09%, or 0.1%, HSA. In some embodiments, the first composition comprises less than about 5%, 4%, 3%, 2%, 1%, 0.5%, 0.06%, or 0.05% HSA. In some embodiments, the first composition comprises about 0.01%, 0.02%, 0.03%, 0.04%, 0.05%, 0.06%, 0.07%, 0.08%, 0.9%, 1%, 2%, 3%, 4%, or 5% HSA. In some embodiments, the first composition comprises about 0.05% HSA.

In some embodiments, the first composition comprises MCDB 131. In some embodiments, the first composition comprises DMEM/F12. In some embodiments, the first composition comprises zinc. In some embodiments, the first composition comprises ZnSO4.

In some embodiments, the first composition comprises at least one metabolite. In some embodiments, the at least one metabolite is glutamate, acetate, β-hydroxybutarate, L-carnitine, taurine, formate, or biotin. In some embodiments, the first composition comprises one, two, three, four, five, six, or seven of glutamate, acetate, β-hydroxybutarate, L-carnitine, taurine, formate, or biotin.

In some embodiments, the second composition comprises at least one amino acid. In some embodiments, the at least one amino acid is alanine, glutamate, glycine, proline, threonine, or tryptophan. In some embodiments, the at least one amino acid is arginine, histidine, lysine, aspartic acid, glutamic acid, serine, asparagine, glutamine, cysteine, selenocysteine, alanine, valine, isoleucine, leucine, methionine, phenylalanine, tyrosine, glutamate, glycine, proline, threonine, or tryptophan.

In some embodiments, the second composition comprises at least one vitamin. In some embodiments, the at least one vitamin is biotin or riboflavin.

In some embodiments, contacting with the second composition comprises contacting the second population of cells with the second composition for about 1 day, 2 days, 3 days, 4 days, 5 days, 6 days, 7 days, or 8 or more days. In some embodiments, the contacting with the second composition comprises contacting the second population of cells with the second composition for about 6 hours, 10 hours, 12 hours, 24 hours, 30 hours, 36 hours, 40 hours, 48 hours, 56 hours, 72 hours, or more hours. In some embodiments, the contacting with the second composition comprises contacting the second population of cells with the second composition for from about 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, 4-5 days, 5-10 days, 5-9 days, 5-8 days, 5-7 days, 5-6 days, 6-10 days, 6-9 days, 6-8 days, 6-7 days, 7-10 days, 7-8 days, 8-10 days, 8-9, days, or 9-10 days. In some embodiments, the contacting with the second composition comprises contacting the second population of cells with the second composition for about 6-96 hours, 6-72 hours, 6-48 hours, 6-24 hours, 6-12 hours, 12-96 hours, 12-72 hours, 12-48 hours, 12-24 hours, 24-96 hours, 24-72 hours, 24-45 hours, 48-96 hours, or 48-72 hours. In some embodiments, the contacting with the second composition comprises contacting the second population of cells with the second composition for about 7 days.

In some embodiments, the second composition does not comprise one or more of a MGLL inhibitor, a TGF-β signaling pathway inhibitor, a thyroid hormone signaling pathway activator, a bone morphogenic protein (BMP) type 1 receptor inhibitor, a Rho-associated coiled-coil containing protein kinase (ROCK) inhibitor, a histone methyltransferase inhibitor, or a protein kinase inhibitor.

In some embodiments, the second composition does not comprise a MGLL inhibitor.

In some embodiments, the second composition does not comprise a TGF-β signaling pathway inhibitor.

In some embodiments, the second composition does not comprise a thyroid hormone signaling pathway activator.

In some embodiments, the second composition does not comprise a bone morphogenic protein (BMP) type 1 receptor inhibitor.

In some embodiments, the second composition does not comprise a Rho-associated coiled-coil containing protein kinase (ROCK) inhibitor.

In some embodiments, the second composition does not comprise a histone methyltransferase inhibitor.

In some embodiments, the second composition does not comprise a protein kinase inhibitor.

In some embodiments, the second composition does not comprise a TGF-β signaling pathway inhibitor, a thyroid hormone signaling pathway activator, a bone morphogenic protein (BMP) type 1 receptor inhibitor, a Rho-associated coiled-coil containing protein kinase (ROCK) inhibitor, a histone methyltransferase inhibitor, and a protein kinase inhibitor.

In some embodiments, the second composition does not comprise a MGLL inhibitor, a TGF-β signaling pathway inhibitor, a thyroid hormone signaling pathway activator, a bone morphogenic protein (BMP) type 1 receptor inhibitor, a Rho-associated coiled-coil containing protein kinase (ROCK) inhibitor, a histone methyltransferase inhibitor, and a protein kinase inhibitor.

In some embodiments, the second composition comprises a lipid. In some embodiments, the lipid is a saturated fatty acid. In some embodiments, the saturated fatty acid is palmitate. In some embodiments, the lipid is a unsaturated fatty acid. In some embodiments, the unsaturated fatty acid is oleic acid, linoleic acid, or palmitoleic acid.

In some embodiments, the second composition comprises a MGLL inhibitor.

In some embodiments, the second composition does not comprise human serum albumin (HSA). In some embodiments, the second composition comprises human serum albumin (HSA). In some embodiments, the second composition comprises about 0.1-5%, 0.1-4%, 0.1-3%, 0.1-2%, 0.1-1%, 0.1-0.5% HSA. In some embodiments, the second composition comprises less than about 5%, 4%, 3%, 2%, 1%, 0.6%, 0.5% HSA. In some embodiments, the second composition comprises about 0.1%, 0.2%, 0.3%, 0.4%, 0.5%, 0.6%, 0.7%, 0.8%, 0.9%, 1%, 2%, 3%, 4%, or 5% HSA. In some embodiments, the second composition comprises about 1% HSA.

In some embodiments, the second composition comprises MCDB 131. In some embodiments, the second composition comprises DMEM/F12.

In some embodiments, the second composition comprises zinc. In some embodiments, the second composition comprises ZnSO4.

In some embodiments, the second composition comprises at least one metabolite. In some embodiments, the at least one metabolite is glutamate, acetate, β-hydroxybutarate, L-carnitine, taurine, formate, or biotin. In some embodiments, the second composition comprises one, two, three, four, five, six, or seven of glutamate, acetate, β-hydroxybutarate, L-carnitine, taurine, formate, or biotin.

In some embodiments, the second composition comprises at least one amino acid. In some embodiments, the at least one amino acid is alanine, glutamate, glycine, proline, threonine, or tryptophan.

In some embodiments, the second composition comprises at least one vitamin. In some embodiments, the at least one vitamin is biotin or riboflavin.

In some embodiments, the dissociating does not comprise subjecting the population of cells to flow cytometry.

Provided herein are methods comprising: (a) obtaining a first population of cells comprising a plurality of cell clusters comprising insulin-positive endocrine cells; (b) dissociating at least a portion of the plurality of cell clusters in the first population of cells in vitro; (c) freezing at least a portion of the first population of cells comprising at least a portion of the dissociated cell clusters; (d) thawing at least a portion of the frozen first population of cells; (e) contacting the at least a portion of the thawed first population of cells with a first composition in vitro, wherein the first composition comprises one, two, three, four, or five of the following agents: a monoglyceride lipase (MGLL) inhibitor, a bone morphogenic protein (BMP) type 1 receptor inhibitor, a Rho-associated coiled-coil containing protein kinase (ROCK) inhibitor, a histone methyltransferase inhibitor, or a protein kinase inhibitor, to obtain a second population of cells comprising a plurality of insulin-positive endocrine cells comprising a plurality of cell clusters; and (f) contacting the second population of insulin-positive endocrine cells in vitro with a second composition, wherein the second composition is different from the first composition, thereby differentiating at least a portion of said second population of insulin-positive endocrine cells into a third population of cells comprising a plurality of β cells comprising a plurality of cell clusters, wherein the third population of cells comprises a higher percentage of viable β cells as compared to a corresponding population of β cells comprising β cells derived from the first population of cells which is not contacted with the first composition.

In some embodiments, the first composition comprises a bone morphogenic protein (BMP) type 1 receptor inhibitor, a Rho-associated coiled-coil containing protein kinase (ROCK) inhibitor, a histone methyltransferase inhibitor, and a protein kinase inhibitor.

In some embodiments, the first composition further comprises a transforming growth factor 3 (TGF-β) signaling pathway inhibitor.

In some embodiments, the first composition further comprises a thyroid hormone signaling pathway activator.

In some embodiments, the first composition further comprises a monoglyceride lipase (MGLL) inhibitor.

Provided herein are methods comprising: (a) obtaining a first population of cells comprising a plurality of cell clusters comprising insulin-positive endocrine cells; (b) dissociating at least a portion of the plurality of cell clusters in the first population of cells in vitro; (c) freezing at least a portion of the first population of cells comprising at least a portion of the dissociated cell clusters; (d) thawing at least a portion of the frozen first population of cells; (e) contacting the at least a portion of the thawed first population of cells with a first composition in vitro, wherein the first composition comprises a transforming growth factor β (TGF-β) signaling pathway inhibitor, a thyroid hormone signaling pathway activator, or both, and one, two, three, four, or five of the following agents: a monoglyceride lipase (MGLL) inhibitor, a bone morphogenic protein (BMP) type 1 receptor inhibitor, a Rho-associated coiled-coil containing protein kinase (ROCK) inhibitor, a histone methyltransferase inhibitor, or a protein kinase inhibitor, to obtain a second population of cells comprising a plurality of insulin-positive endocrine cells comprising a plurality of cell clusters; and (f) contacting the second population of insulin-positive endocrine cells in vitro with a second composition, wherein the second composition is different from the first composition, thereby differentiating at least a portion of said second population of insulin-positive endocrine cells into a third population of cells comprising a plurality of β cells comprising a plurality of cell clusters, wherein the third population of cells comprises a higher percentage of viable β cells as compared to a corresponding population of β cells comprising β cells derived from the first population of cells which is not contacted with the first composition.

In some embodiments, the first composition comprises a monoglyceride lipase (MGLL) inhibitor, a bone morphogenic protein (BMP) type 1 receptor inhibitor, a Rho-associated coiled-coil containing protein kinase (ROCK) inhibitor, a histone methyltransferase inhibitor, and a protein kinase inhibitor.

Provided herein are methods comprising: (a) obtaining a first population of cells comprising a plurality of cell clusters comprising insulin-positive endocrine cells; (b) dissociating at least a portion of the plurality of cell clusters in the first population of cells in vitro; (c) contacting the first population of cells comprising at least a portion of the dissociated cell clusters with a first composition in vitro, to obtain a second population of cells comprising a plurality of cells clusters comprising a plurality of insulin-positive endocrine cells; and (d) contacting the second population of insulin-positive endocrine cells in vitro with a second composition, wherein the second composition is different from the first composition and the second composition comprises at least one metabolite, thereby differentiating at least a portion of said second population of insulin-positive endocrine cells into a third population of cells comprising a plurality of β cells, wherein the third population of cells comprises a higher percentage of viable cells as compared to a corresponding population of cells comprising β cells derived from the first population of cells which is not contacted with the second composition.

Provided herein are methods comprising: (a) obtaining a first population of cells comprising a plurality of cell clusters comprising insulin-positive endocrine cells; (b) dissociating at least a portion of the plurality of cell clusters in the first population of cells in vitro; (c) contacting the first population of cells comprising at least a portion of the dissociated cell clusters with a first composition in vitro, to obtain a second population of cells comprising a plurality of cells clusters comprising a plurality of insulin-positive endocrine cells; and (d) contacting the second population of insulin-positive endocrine cells in vitro with a second composition, wherein the second composition is different from the first composition and the second composition comprises at least one metabolite, thereby differentiating at least a portion of said second population of insulin-positive endocrine cells into a third population of cells comprising a plurality of β cells, wherein the plurality of β cells exhibit improved glucose stimulated insulin secretion relative to a corresponding population of cells comprising β cells derived from the first population of cells which is not contacted with the second composition.

In some embodiments, the at least one metabolite is glutamate, acetate, β-hydroxybutarate, L-carnitine, taurine, formate, or biotin.

In some embodiments, the second composition comprises at least two, three, four, five, six, or seven of the following metabolites glutamate, acetate, β-hydroxybutarate, L-carnitine, taurine, formate, or biotin.

In some embodiments, the second composition comprises DMEM/F12.

In some embodiments, the second composition comprises from about 0.05-2% HSA.

In some embodiments, the second composition comprises about 1% HSA.

In some embodiments, the second composition comprises zinc.

In some embodiments, the second composition comprises ZnSO4.

In some embodiments, the second composition comprises at least one amino acid.

In some embodiments, the at least one amino acid is alanine, glutamate, glycine, proline, threonine, or tryptophan.

In some embodiments, the second composition comprises at least one vitamin.

In some embodiments, the at least one vitamin is biotin or riboflavin.

In some embodiments, the plurality of β cells exhibit improved glucose stimulated insulin secretion relative to a corresponding population of cells comprising β cells derived from the first population of cells which is not contacted with the second composition.

In some embodiments, the third population of cells comprises a higher percentage of viable cells as compared to a corresponding population of cells comprising β cells derived from the first population of cells which is not contacted with the second composition.

In some embodiments, the third population of cells comprises a plurality of cell clusters each with a diameter of about 50-150 microns. In some embodiments, the third population of cells comprises a plurality of cell clusters each with a diameter of about 100 microns. In some embodiments, the third population of cells comprises a plurality of cells clusters, wherein at least 50%, 60%, 70%, 75%, 80%, 90%, or 95% of the cell clusters have a diameter of about 100 microns, in the absence of a selection step. In some embodiments, the third population of cells comprises a plurality of cells clusters, wherein at least 50%, 60%, 70%, 75%, 80%, 90%, or 95% of the cell clusters have a diameter of about 50-150 microns, 75-125 microns, 80-120 microns, or 90-110 microns, in the absence of a selection step.

In some embodiments, the first composition comprises at least one of the following agents: a monoglyceride lipase (MGLL) inhibitor, a bone morphogenic protein (BMP) type 1 receptor inhibitor, a Rho-associated coiled-coil containing protein kinase (ROCK) inhibitor, a histone methyltransferase inhibitor, or a protein kinase inhibitor.

In some embodiments, the method further comprises: (a) freezing at least a portion of the first population of cells comprising at least a portion of the dissociated cell clusters; (b) thawing at least a portion of the frozen first population of cells; (c) contacting at least a portion of the first population of thawed cells in vitro with said first composition.

Provided herein are compositions comprising at least a portion of the second population of cells comprising insulin-positive endocrine cells described herein or made by a method described herein.

Provided herein are compositions comprising at least a portion of the third population of cells of β cells described herein or made by a method described herein.

Provided herein are compositions comprising at least a portion of the third population of cells of β cells described herein or made by a method described herein and at least a portion of the second population of cells comprising insulin-positive endocrine cells described herein or made by a method described herein.

Provided herein are devices comprising a composition of β cells described herein or made by a method described herein.

Provided herein are devices comprising a composition of insulin-positive endocrine cells described herein or made by a method described herein.

Provided herein are devices comprising a composition of insulin-positive endocrine cells and β cells described herein or made by a method described herein.

Provided herein are methods of treating a subject with a disease characterized by high blood sugar levels over a prolonged period of time (e.g., diabetes), the method comprising administering a composition of cells described herein or made by a method described herein, to the subject.

Provided herein are methods of treating a subject with a disease characterized by high blood sugar levels over a prolonged period of time (e.g., diabetes), the method comprising administering a composition of cells described herein or made by a method described herein, to the subject.

Provided herein are methods of treating a subject with a disease characterized by high blood sugar levels over a prolonged period of time (e.g., diabetes), the method comprising administering a composition of cells described herein or made by a method described herein, to the subject.

Provided herein are methods of treating a subject with a disease characterized by high blood sugar levels over a prolonged period of time (e.g., diabetes), the method comprising implanting a device of described herein into the subject.

Provided herein are methods of treating a subject with a disease characterized by high blood sugar levels over a prolonged period of time (e.g., diabetes), the method comprising implanting a device described herein into the subject.

Provided herein are methods of treating a subject with a disease characterized by high blood sugar levels over a prolonged period of time (e.g., diabetes), the method comprising implanting a device described herein into the subject.

In some embodiments, wherein the disease is diabetes. In some embodiments, the disease is type I diabetes. In some embodiments, the disease is type II diabetes.

Provided herein are compositions comprising isolated insulin-positive endocrine cells that have been contacted with an agent that inhibits expression or function of monoglyceride lipase (MGLL) in vitro and exhibit a decreased conversion rate of monoglycerides to free fatty acids compared to a corresponding population of isolated insulin-positive endocrine cells that have not been contacted with the agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit an increased ratio of monoglycerides to free fatty acids compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit a decreased ratio of free fatty acids to monoglycerides compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit a decreased level of free fatty acids compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit an increased level of monoglycerides compared to a corresponding population of isolated insulin-positive endocrine cells that have not been contacted with an agent that inhibits expression or function of monoglyceride lipase (MGLL) in vitro.

Provided herein are compositions comprising a population of insulin-positive endocrine cells and an agent that inhibits the conversion of monoglycerides to free fatty acids.

In some embodiments, said agent inhibits the expression or function of monoglyceride lipase (MGLL).

Provided herein are compositions comprising a population of insulin-positive cells and an agent inhibits the expression or function of monoglyceride lipase (MGLL). In some embodiments, said agent that inhibits expression or function of monoglyceride lipase (MGLL) is JJKK048, KML29, NF1819, JW642, JZL184, JZL195, JZP361, pristimerin, or URB602.

Provided herein are compositions comprising a population of β cells that have been contacted in vitro with at least one agent selected from the group consisting of glutamate, acetate, β-hydroxybutarate, L-carnitine, taurine, formate, or biotin, wherein said population of β cells exhibit increased glucose stimulated insulin secretion compared to a corresponding population of β cells that have not been contacted with said at least one agent. In some embodiments, said population of cells that have been contacted with at least two, three, four, five, six, or seven of the agents selected from the group consisting of glutamate, acetate, β-hydroxybutarate, L-carnitine, taurine, formate, or biotin.

Provided herein are compositions comprising a population of β cells and at least one, two, three, four, five, six, or seven of the agents selected from the group consisting of glutamate, acetate, 3-hydroxybutarate, L-carnitine, taurine, formate, or biotin.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 is a graph showing the percent of stage 6 viable cells recovered post thaw of cryopreserved stage 5 cells using a thaw medium comprising DMEM F12 and 1% human serum albumin (HSA).

FIG. 2 is an illustration showing an outline of one of the experimental protocols described in Example 1.

FIG. 3 is a bar graph showing the aggregate percent of cells recovered from seeded cells at day 4 of stage 6 (S6d4). The cells were cultured in stage 6 (S6) medium, modified stage 5 (S5) medium with XXI, or modified stage 5 (S5) medium without XXI as indicated, with indicated supplements, i.e. glucose (Glc) and pyruvate (Pyr).

FIG. 4 is a bar graph showing the percent of CHGA positive cells recovered at day 4 of stage 6 (S6d4). The cells were cultured in stage 6 (S6) medium, modified stage 5 (S5) medium with XXI, or modified stage 5 (S5) medium without XXI as indicated, with indicated supplements, i.e. glucose (Glc) and pyruvate (Pyr).

FIG. 5A is a FACS plot showing the percentage of stem cell derived β cells (Nkx6.1/Isl1 double positive cells) at day 4 of stage 6 (S6d4). The cells were cultured in stage 6 (S6) medium, modified stage 5 (S5) medium with XXI, or modified stage 5 (S5) medium without XXI as indicated.

FIG. 5B is a table showing the percent of stem cell derived β cells (Nkx6.1/Isl1 double positive cells) at day 4 of stage 6 (S6d4). The cells were cultured in stage 6 (S6) medium, modified stage 5 (S5) medium with XXI, or modified stage 5 (S5) medium without XXI as indicated, with indicated supplements, i.e. glucose (Glc) and pyruvate (Pyr).

FIG. 6 is a bar graph showing the fold improvement in the recovery of stem cell derived β cells at day 4 of stage 6 (S6d4). The cells were cultured in stage 6 (S6) medium, modified stage 5 (S5) medium with XXI, or modified stage 5 (S5) medium without XXI as indicated, with indicated supplements, i.e. glucose (Glc) and pyruvate (Pyr).

FIG. 7 is a bar graph showing the glucose stimulated insulin secretion (GSIS) of the cells at day 11 of stage 6 (S6d11). GSIS is measured by the level of human C-peptide (pM) per 1000 cells. The cells were cultured in either stage 6 (S6) medium or modified stage 5 (S5) medium during days 1-4 of stage 6 (approximately 72 hours) as indicated, and cultured in stage 6 (S6) medium for days 4-7 or 4-11 as indicated.

FIG. 8 shows microscopy images of cultured cells at day 7 of stage 6 (S6d7). The cells were cultured in stage 6 (S6) medium, modified stage 5 (S5) medium with XXI, or modified stage 5 (S5) medium without XXI, for days 1-4 of stage 6 (approximately 72 hours) as indicated. The cells were subsequently cultured in S6 (S6) medium for days 4-7 of stage 6. As indicated, the modified stage 5 base medium was MCDB 131 with 0.05% HSA. The stage 6 base medium was DMEM F12 with 1% HSA.

FIG. 9 is a microscopy image of stage 6 cell clusters produced from cells cultured in stage 6 (S6) medium with 1.0% HSA for the length of stage 6 (left) or modified stage 5 (S5) medium with 0.05% HSA for days 1-4 (approximately 72 hours) of stage 6 (right).

FIG. 10A is a bar graph showing the level of human C-peptide (pM) per 1000 cells at day 11 of stage 6 (S6d11). The cells were cultured in either stage 6 (S6) medium or modified stage 5 (S5) medium during days 1-4 (approximately 72 hours) of stage 6, as indicated, and subsequently cultured in stage 6 (S6) medium for days 4-11 of stage 6.

FIG. 10B is a FACS plot showing the percentage of stem cell derived β cells (Nkx6.1/Isl1 double positive cells) at day 4 of stage 6 (S6d4). The cells were cultured in either stage 6 (S6) medium or modified stage 5 (S5) medium during days 1-4 (approximately 72 hours) of stage 6, as indicated, and subsequently cultured in stage 6 (S6) medium for days 4-11 of stage 6.

FIG. 11 is a graph showing the percent of cells recovered from seed when the cells are cultured for days 1-4 (approximately 72 hours) of stage 6 in stage 6 medium (left) or modified stage 5 medium (minus XXI) (right).

FIG. 12 a table depicting a summary of the results of Example 1, showing the effect of stage 6 medium or stage 5 day 6 medium (S5d6 medium) for the culture of cells in days 1-4 (approximately 72 hours) of stage 6, on total cell recovery, yield of cells, the percentage of β cells (composition), insulin content of the β cells recovered, and GSIS of the cells.

FIG. 13 is an illustration showing an outline of one of the experimental protocols described in Example 2.

FIG. 14 is a bar graph showing the percentage of total cells and the percentage of stem cell derived β cells recovered at day 4 of stage 6 (S6d4). The cells were cultured in medium comprising 1% HSA or 0.05% HSA as indicated with the indicated modified stage 5 medium factors.

FIG. 15 is a bar graph showing the number of stem cell derived β cells at day 4 of stage 6 (S6d4). The cells were cultured in medium comprising 1% HSA or 0.05% HSA as indicated with the indicated modified stage 5 medium factors.

FIG. 16 is a bar graph showing the number of stem cell derived β cells at day 12 of stage 6 (S6d12). The cells were cultured in medium comprising 1% HSA or 0.05% HSA as indicated with the indicated modified stage 5 medium factors.

FIG. 17 is a bar graph showing the level of human C-peptide (pM) per 1000 cells at day 12 of stage 6 (S6d12). The cells were cultured in medium comprising 1% HSA or 0.05% HSA as indicated with the indicated modified stage 5 medium factors.

FIG. 18A is a bar graph showing the glucose stimulated insulin secretion (GSIS) of cells at day 12 of stage 6 (S6d12). GSIS was measured as the level of human C-peptide (pM) per 1000 cells after glucose stimulation. The cells were cultured in stage 6 (S6) medium+1% HSA for days 1-12 of stage 6.

FIG. 18B is a bar graph showing the glucose stimulated insulin secretion (GSIS) of cells at day 12 of stage 6 (S6d12). GSIS was measured as the level of human C-peptide (pM) per 1000 cells after glucose stimulation. The cells were cultured in modified stage 5 (S5) medium+0.05% HSA for days 1-4 (approximately 72 hours) of stage 6, and base stage 5 (S5) medium+0.05% HSA for days 4-12 of stage 6.

FIG. 19 is a table illustrating the results of Example 2, showing the effect of stage 6 medium or stage 5 day 6 medium (S5d6 medium) for the culture of cells in days 1-4 of stage 6, on total cell recovery, yield of cells, composition of the cells recovered, insulin content of the cells, and GSIS of the cells.

FIG. 20 is an illustration showing an outline of one of the experimental protocols described in Example 3.

FIG. 21 is a bar graph showing the percent of SC-β cells (Nkx6.1/Isl1 double positive cells) recovered with stage 5 cells cultured in the indicated medium for days 1-4 (approximately 72 hours) of stage 6.

FIG. 22 is a bar graph showing the number of SC-β cells at day 10 of stage 6 (S6d10), where the stage 5 cells have been cultured in the indicated culture medium for days 1-4 (approximately 72 hours) of stage 6.

FIG. 23 shows a microscopy image day 4 stage 6 (S6d4) cell clusters produced from stage 5 cells cultured in modified stage 5 (S5) medium with 0.5% HSA (left), modified stage 5 (S5) medium with 0.5% HSA and palmitate (center), or modified stage 5 (S5) medium with 0.5% HSA and linoleic acid (right) during days 1-4 (approximately 72 hours) of stage 6.

FIG. 24A is a bar graph showing the number of cells at day 10 of stage 6 (S6d10), wherein the stage 5 cells have been cultured in the indicated medium during days 1-4 (approximately 72 hours) of stage 6 and stage 6 (S6) medium during days 4-10 of stage 6.

FIG. 24B is a bar graph showing the number of stem cell derived β cells (SC-β) cells at day 10 of stage 6 (S6d10), wherein the stage 5 cells have been cultured in the indicated medium during days 1-4 (approximately 72 hours) of stage 6 and stage 6 (S6) medium during days 4-10 of stage 6.

FIG. 25 is a bar graph showing the C-peptide content across 16 different samples of stage 6 day 14 (S6d14) cells cultured in a medium comprising an MGLL inhibitor (1 μM JJKK 048, 10 μM KML-29, 10 μM NF1819).

FIG. 26A is a bar graph showing the glucose stimulated insulin secretion (GSIS) of day 10 stage 6 (S6d10) cells that were cultured during days 1-10 of stage 6 in Stage 6 (S6) medium.

FIG. 26B is a bar graph showing the glucose stimulated insulin secretion (GSIS) of day 10 stage 6 (S6d10) cells that were cultured during days 1-4 of stage 6 in modified stage 5 (S5) medium with 0.05% HSA, and cultured in stage 6 (S6) medium during days 4-10.

FIG. 27A is a bar graph showing the glucose stimulated insulin secretion (GSIS) of day 10 stage 6 (S6d10) cells that were cultured during days 1-4 (approximately 72 hours) of stage 6 in modified stage 5 (S5) medium with 0.5% HSA.

FIG. 27B is a bar graph showing the glucose stimulated insulin secretion (GSIS) of day 10 stage 6 (S6d10) cells that were cultured during days 1-4 of stage 6 in modified stage 5 (S5) medium with 0.5% HSA and palmitate.

FIG. 28 shows a series of FACS plots showing the percentage of stem cell derived β cells (SC-β) cells (Nkx6.1/Isl1 double positive cells) recovered in stage 6 when the cells have been cultured in the modified S5d6 medium and an MGLL inhibitors (days 1-10 of stage 6) (two right plots), compared to cells cultured in S6 control medium (left) or modified S5 medium with factors (center).

FIG. 29 is a table depicting a summary of the results of Example 3 and Example 4, showing the effect of 1% HSA, fatty acids, and MGLL inhibitor supplementation (in stage 5 medium MCBD with factors and 0.05% HSA) in the culture of cells in days 1-4 (approximately 72 hours) of stage 6, on yield of stem cell derived β cells, the percent of stem cell derived β cells (composition), the insulin content of the recovered stem cell derived β cells (content), and the glucose stimulated insulin secretion (GSIS) of the stem cell derived β cells.

FIG. 30 shows a depiction of stage 6, wherein day 1 (D1) starts with the thaw of cryopreserved stage 5 cells, day 4 (D4) is the process intermediate, day 7 (D7) is drug substance, and day 11 (D11) are cells with GSIS activity and insulin content. The DS2 medium is an optional stage 6 media used throughout days 1-11 that contains DMDM/F12 and 1% HSA. The DS3 medium is a second optional stage 6 medium. For days 1-4 of stage 6, the DS3 medium contains MCDB 131 supplemented with S5d6 factors (Alk5i (10 μM), GC-1 (1 μM), LDN-193189 (100 nM), thiazovinin (2.5 μM), SSP (3 nM), DZNEP (100 nM)), 0.05% HSA, ITS-X, glutamax, VitC, and optionally additional agents such as lipids and MGLL inhibitors. For days 5-11 of stage 6, the DS3 medium contains MCDB 131 supplemented with 0.05% HSA.

FIG. 31 shows a table reciting the composition of the stage 6 media DS2 and the stage 6 media DS3.

FIG. 32 shows a table illustrating the recovery and functional properties of the DS2 stage 6 media compared to DS3 stage 6 media. As shown, the DS3 media improved cell recovery at day 4 of stage 6 (S6d4) and day 7 of stage 6 (S6d7) compared to the DS2 stage 6 media. The DS3 stage 6 media does not improve cell recovery at day 11 of stage 6 (S6d11) compared to the DS2 stage 6 media, nor does it improve the insulin content of the cells or percentage of SC-β cells. The DS3 stage 6 media decreases glucose stimulated insulin secretion (GSIS) function, compared to the DS2 stage 6 media.

FIG. 33 is a bar graph showing the percent of cells recovered from viable seeded cells at S6d4, S6d7, or S6d11 from either DS2 cultured stage 6 cells or DS3 cultured stage 6 cells. The results shows on average an improvement in percent cell recovery through stage 6 with the use of DS3 media.

FIG. 34A is a bar graph showing the percent of SC-β cells (Nkx6.1/Isl1 cells) at S6d4 or S6d11 from either DS2 cultured stage 6 cells or DS3 cultured stage 6 cells. The results shows the DS3 stage 6 media retains similar percent SC-β cell to that of the DS2 stage 6 media.

FIG. 34B shows the percent endocrine cells (chga+ cells) at S6d4 or S6d11 from either DS2 cultured stage 6 cells or DS3 cultured stage 6 cells. The results show the DS3 stage 6 media retains similar percent of endocrine cells to that of the DS2 stage 6 media.

FIG. 35 is a bar graph showing the total level of insulin content of SC-islets at S6d4 or S6d11 from either DS2 cultured stage 6 cells or DS3 cultured stage 6 cells. The results shows the cells cultured in DS3 stage 6 media have similar insulin content to cells cultured in the DS2 stage 6 media.

FIG. 36 is a bar graph showing the glucose stimulated insulin secretion of cells cultured in DS2 stage 6 media or DS3 stage 6 media and either high glucose stimulation (HG), low glucose stimulation (LG), or KLC treatment (positive control). The results shows that the DS3 media generates SC-islets with less GSIS function compared to the DS2 stage 6 media.

FIG. 37 is a line graph showing the blood glucose level (mg/dL) of mice implanted with a device containing SC-islets cultured in DS3 stage 6 media or DS2 stage 6 media. The results show that DS2 and DS3 SC-islets are capable of functioning in vivo.

FIG. 38 is a line graph showing the blood glucose level (mg/dL) of mice implanted with a device containing SC-islets cultured in DS3 stage 6 media and indicate control of blood glucose in vivo.

FIG. 39 shows a third stage 6 media (“DS6”) and the components compared to the DS2 stage 6 media and the DS3 stage 6 media.

FIG. 40 is a scatter plot showing the ratio of amino acids in human plasma-like media (HPLM) to MCDB 131 (circle), HPLM to DMEM/F12 (square), and HPLM to CRML (triangle). The results show that the MCDB 131 media contains lower levels of specific amino acids, including e.g., alanine, glutamate, and glycine.

FIG. 41 is a scatter plot showing the ration of vitamins in human plasma-like media (HPLM) to MCDB 131 (circle), HPLM to DMEM/F12 (square), and HPLM to CRML (triangle). The results show that the MCDB 131 media contains lower levels of specific vitamins, including e.g., biotin and riboflavin.

FIG. 42 is a table showing the total number of viable cells at S6d4, S6d7, and S6d11 cultured in DS2 stage 6 media, DS3 stage 6 media, or DS3 stage 6 media with ZnSO4. The results show that the inclusion of ZnSO4 in DS3 media greatly improves S6d4 viable cell number, but cell loss remains postS6d4.

FIG. 43 is a bar graph showing the percent of cells recovered at S6d4, S6d7, and S6d11, wherein the cells are cultured in DS2, DS3, or DS3 plus ZnSO4. The results show that the inclusion of ZnSO4 in DS3 media greatly improves S6d4 cell recovery, but cell loss remains postS6d4.

FIG. 44 is a FACS plot showing the number of β cells (chga+/Nkx6.1+) recovered at S6d4 using DS3 stage 6 media supplemented with ZnSO4.

FIG. 45A is a table showing the total number of viable cells at S6d11 cultured in DS2 stage 6 media, DS3 stage 6 media, DS3 stage 6 media supplemented with metabolites, or MCDB 131 media without vitamins but supplemented with amino acids and metabolites. The results show that the inclusion of metabolites and vitamins in DS3 media greatly improves S6d11 viable cell number.

FIG. 45B is a bar graph showing the percent of cells recovered at S6d4 or S6d11 cultured in DS2, DS3, or DS3 supplemented with metabolites. The results show that the inclusion of metabolites in DS3 media greatly improves cell recovery at S6d11.

FIG. 46 is a bar graph showing the percent of SC-β cells (Nkx6.1/Isl1+ cells) at S6d11 cultured in DS2 media, DS3 media with the recited supplements, and MCDB 131 media with the recited supplements. The data shows that additional media supplements (e.g., vitamins, amino acids, metabolites, and lipids) can improve the percentage of SC-β cells through S6 d11.

FIG. 47 is a bar graph showing the glucose stimulated insulin secretion (GSIS) of cells cultures in DS2, DS3, or MCDB 131 media with the indicated supplements. The results show that the supplements are insufficient to improve the high glucose GSIS function of cells cultured in MCDB 131 to reach the level of GSIS in DS2 cultured cells, although it improves some aspects such as the magnitude of KCL-induced insulin secretion.

FIG. 48A is a bar graph showing the number of viable cells recovered at S6d7 and S6d11 from DS2, DS3, DS6 (without metabolites), or DS6 (with metabolites) cultures. The data show the DS6 media using DMEM/F12 further improves viable cell count throughout stage 6.

FIG. 48B is a bar graph showing the percent of cells recovered at S6d7 and S6d11 from DS2, DS3, DS6 (without metabolites), or DS6 (with metabolites) cultures. The data show the DS6 media using DMEM/F12 further improves cell recovery throughout stage 6.

FIG. 49 is a microscopy image of SC-islet clusters at S6d7 and S6d11 from DS2, DS3, or DS6 (with metabolites) cultures. The images show that the DS6 clusters exhibit more homogeneity throughout stage 6.

FIG. 50 shows a series of area graphs showing the frequency and cluster size of cell clusters at S6d4, S6d7, and S6d11 from DS2, DS3, and DS6 cultures. The results show that the DS6 clusters are smaller and exhibit greater homogeneity at S6d11.

FIG. 51 is a bar graph showing the GSIS function of S6d11 cells from DS2 culture.

FIG. 52 is a bar graph showing the GSIS function of S6d11 cells from DS3 culture. The results show that SC-islets in DS3 media do not exhibit GSIS function up to that of DS2 (compare to FIG. 51).

FIG. 53 is a bar graph showing the GSIS function of S6d11 cells from DS6 (without metabolites) culture. The results show that SC-islets in DS6 (without metabolites) media exhibit improved GSIS function (compare to FIG. 51).

FIG. 54 is a bar graph showing the GSIS function of S6d11 cells from DS6 (with metabolites) culture. The results show that SC-islets in DS6 media exhibit identical GSIS function to DS2 cultures (compare to FIG. 51).

FIG. 55 is a table showing the effect of the DS5 stage 6 media. The DS6 stage 6 media shows improved cell recovery at S6d11 compared to DS3, and improved GSIS function compared to the DS2 media.

FIG. 56 is an area graph showing the cluster size SC-islets cultured in stage 5 media with and without lipid supplementation. The results show that cluster size increases with lipid supplementation.

FIG. 57 is a bar graph showing the percent of blood glucose control in mice implanted with SC-islets cultured in. The results show that the modified formulation not only improves post-cryopreservation cluster re-aggregation, but the cells also exhibit efficacy in vivo.

### DETAILED DESCRIPTION OF THE DISCLOSURE

Provided herein are, inter alia, methods of increasing re-aggregation efficiency, optimizing cluster size, and function of SC-β cells in vitro. The specification discloses the identification of novel signaling requirements that not only enhance CDGA positive endocrine cell re-aggregation efficiency, but also improve SC-islet composition and cluster size, and SC-β cell function in vitro. The novel methods can be employed in the large scale manufacture of SC-islets for human therapeutic use.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### Definitions

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “diabetes” and its grammatical equivalents as used herein can refer to is a disease characterized by high blood sugar levels over a prolonged period. For example, the term “diabetes” and its grammatical equivalents as used herein can refer to all or any type of diabetes, including, but not limited to, type 1, type 2, cystic fibrosis-related, surgical, gestational diabetes, and mitochondrial diabetes. In some cases, diabetes can be a form of hereditary diabetes.

The term “endocrine cell(s),” if not particularly specified, can refer to hormone-producing cells present in the pancreas of an organism, such as “islet”, “islet cells”, “islet equivalent”, “islet-like cells”, “pancreatic islets” and their grammatical equivalents. In an embodiment, the endocrine cells can be differentiated from pancreatic progenitor cells or precursors. Islet cells can comprise different types of cells, including, but not limited to, pancreatic α cells, pancreatic β cells, pancreatic δ cells, pancreatic F cells, and/or pancreatic E cells. Islet cells can also refer to a group of cells, cell clusters, or the like.

The terms “progenitor” and “precursor” cell are used interchangeably herein and refer to cells that have a cellular phenotype that is more primitive (e.g., is at an earlier step along a developmental pathway or progression than is a fully differentiated cell) relative to a cell which it can give rise to by differentiation. Often, progenitor cells can also have significant or very high proliferative potential. Progenitor cells can give rise to multiple distinct differentiated cell types or to a single differentiated cell type, depending on the developmental pathway and on the environment in which the cells develop and differentiate.

A “precursor thereof” as the term related to an insulin-positive endocrine cell can refer to any cell that is capable of differentiating into an insulin-positive endocrine cell, including for example, a pluripotent stem cell, a definitive endoderm cell, a primitive gut tube cell, a pancreatic progenitor cell, or endocrine progenitor cell, when cultured under conditions suitable for differentiating the precursor cell into the insulin-positive endocrine cell.

The term “exocrine cell” as used herein can refer to a cell of an exocrine gland, i.e. a gland that discharges its secretion via a duct. In particular embodiments, an exocrine cell can refer to a pancreatic exocrine cell, which is a pancreatic cell that can produce enzymes that are secreted into the small intestine. These enzymes can help digest food as it passes through the gastrointestinal tract. Pancreatic exocrine cells are also known as islets of Langerhans, which can secrete two hormones, insulin and glucagon. A pancreatic exocrine cell can be one of several cell types; α-2 cells (which can produce the hormone glucagon); or β cells (which can manufacture the hormone insulin); and α-1 cells (which can produce the regulatory agent somatostatin). Non-insulin-producing exocrine cells, as the term is used herein, can refer to α-2 cells or α-1 cells. The term pancreatic exocrine cells encompasses “pancreatic endocrine cells” which can refer to a pancreatic cell that produces hormones (e.g., insulin (produced from β cells), glucagon (produced by alpha-2 cells), somatostatin (produced by delta cells) and pancreatic polypeptide (produced by F cells) that are secreted into the bloodstream.

The terms “stem cell-derived β cell,” “SC-β cell,” “functional β cell,” “functional pancreatic β cell,” “mature SC-β cell,” and their grammatical equivalents can refer to cells (e.g., non-native pancreatic β cells) that display at least one marker indicative of a pancreatic β cell (e.g., PDX-1 or NKX6.1), expresses insulin, and display a glucose stimulated insulin secretion (GSIS) response characteristic of an endogenous mature β cell. In some embodiments, the terms “SC-β cell” and “non-native β cell” as used herein are interchangeable. In some embodiments, the “SC-β cell” comprises a mature pancreatic cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). In some embodiments, the SC-β cells exhibit a response to multiple glucose challenges (e.g., at least one, at least two, or at least three or more sequential glucose challenges). In some embodiments, the response resembles the response of endogenous islets (e.g., human islets) to multiple glucose challenges. In some embodiments, the morphology of the SC-β cell resembles the morphology of an endogenous β cell. In some embodiments, the SC-β cell exhibits an in vitro GSIS response that resembles the GSIS response of an endogenous β cell. In some embodiments, the SC-β cell exhibits an in vivo GSIS response that resembles the GSIS response of an endogenous β cell. In some embodiments, the SC-β cell exhibits both an in vitro and in vivo GSIS response that resembles the GSIS response of an endogenous β cell. The GSIS response of the SC-β cell can be observed within two weeks of transplantation of the SC-β cell into a host (e.g., a human or animal). In some embodiments, the SC-β cells package insulin into secretory granules. In some embodiments, the SC-β cells exhibit encapsulated crystalline insulin granules. In some embodiments, the SC-β cells exhibit a stimulation index of greater than 1. In some embodiments, the SC-β cells exhibit a stimulation index of greater than 1.1. In some embodiments, the SC-β cells exhibit a stimulation index of greater than 2. In some embodiments, the SC-β cells exhibit cytokine-induced apoptosis in response to cytokines. In some embodiments, insulin secretion from the SC-β cells is enhanced in response to known antidiabetic drugs (e.g., secretagogues). In some embodiments, the SC-β cells are monohormonal. In some embodiments, the SC-β cells do not abnormally co-express other hormones, such as glucagon, somatostatin or pancreatic polypeptide. In some embodiments, the SC-β cells exhibit a low rate of replication. In some embodiments, the SC-β cells increase intracellular Ca2+ in response to glucose.

As used herein, the term “insulin producing cell” and its grammatical equivalent refer to a cell differentiated from a pancreatic progenitor, or precursor thereof, which secretes insulin. An insulin-producing cell can include pancreatic β cell as that term is described herein, as well as pancreatic β-like cells (e.g., insulin-positive, endocrine cells) that synthesize (e.g., transcribe the insulin gene, translate the proinsulin mRNA, and modify the proinsulin mRNA into the insulin protein), express (e.g., manifest the phenotypic trait carried by the insulin gene), or secrete (release insulin into the extracellular space) insulin in a constitutive or inducible manner. A population of insulin producing cells e.g., produced by differentiating insulin-positive, endocrine cells or a precursor thereof into SC-β cells according to the methods of the present disclosure can be pancreatic β cell or (β-like cells (e.g., cells that have at least one, or at least two least two) characteristic of an endogenous β cell and exhibit a glucose stimulated insulin secretion (GSIS) response that resembles an endogenous adult β cell. The population of insulin-producing cells, e.g. produced by the methods as disclosed herein can comprise mature pancreatic β cell or SC-β cells, and can also contain non-insulin-producing cells (e.g., cells of cell like phenotype with the exception they do not produce or secrete insulin).

The terms “insulin-positive β-like cell,” “insulin-positive endocrine cell,” and their grammatical equivalents can refer to cells (e.g., pancreatic endocrine cells) that displays at least one marker indicative of a pancreatic β cell and also expresses insulin but lack a glucose stimulated insulin secretion (GSIS) response characteristic of an endogenous β cell.

The term “β cell marker” refers to, without limitation, proteins, peptides, nucleic acids, polymorphism of proteins and nucleic acids, splice variants, fragments of proteins or nucleic acids, elements, and other analyte which are specifically expressed or present in pancreatic β cells. Exemplary β cell markers include, but are not limited to, pancreatic and duodenal homeobox 1 (Pdx1) polypeptide, insulin, c-peptide, amylin, E-cadherin, Hnf3β, PCI/3, B2, Nkx2.2, GLUT2, PC2, ZnT-8, ISL1, Pax6, Pax4, NeuroD, 1 Inf1b, Hnf-6, Hnf-3beta, and MafA, and those described in Zhang et al., Diabetes. 50(10):2231-6 (2001). In some embodiment, the β cell marker is a nuclear 3-cell marker. In some embodiments, the β cell marker is Pdx1 or PH3.

The term “pancreatic endocrine marker” can refer to without limitation, proteins, peptides, nucleic acids, polymorphism of proteins and nucleic acids, splice variants, fragments of proteins or nucleic acids, elements, and other analyte which are specifically expressed or present in pancreatic endocrine cells. Exemplary pancreatic endocrine cell markers include, but are not limited to, Ngn-3, NeuroD and Islet-1.

The term “pancreatic progenitor,” “pancreatic endocrine progenitor,” “pancreatic precursor,” “pancreatic endocrine precursor” and their grammatical equivalents are used interchangeably herein and can refer to a stem cell which is capable of becoming a pancreatic hormone expressing cell capable of forming pancreatic endocrine cells, pancreatic exocrine cells or pancreatic duct cells. These cells are committed to differentiating towards at least one type of pancreatic cell, e.g. β cells that produce insulin; a cells that produce glucagon; δ cells (or D cells) that produce somatostatin; and/or F cells that produce pancreatic polypeptide. Such cells can express at least one of the following markers: NGN3, NKX2.2, NeuroD, ISL-1, Pax4, Pax6, or ARX.

The term “Pdx1-positive pancreatic progenitor” as used herein can refer to a cell which is a pancreatic endoderm (PE) cell which has the capacity to differentiate into SC-β cells, such as pancreatic β cells. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. In some cases, a Pdx1-positive pancreatic progenitor cell lacks expression of NKX6.1. In some cases, a Pdx1-positive pancreatic progenitor cell can also be referred to as Pdx1-positive, NKX6.1-negative pancreatic progenitor cell due to its lack of expression of NKX6.1. In some cases, the Pdx1-positive pancreatic progenitor cells can also be termed as “pancreatic foregut endoderm cells.”

The term “Pdx1-positive, NKX6-1-positive pancreatic progenitor” as used herein can refer to a cell which is a pancreatic endoderm (PE) cell which has the capacity to differentiate into insulin-producing cells, such as pancreatic β cells. A Pdx1-positive, NKX6-1-positive pancreatic progenitor expresses the markers Pdx1 and NKX6-1. Other markers include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR. As used herein, the terms “NKX6.1” and “NKX6-1” are equivalent and interchangeable. In some cases, the Pdx1-positive, NKX6-1-positive pancreatic progenitor cells can also be termed as “pancreatic foregut precursor cells.”

The term “Ngn3-positive endocrine progenitor” as used herein can refer to precursors of pancreatic endocrine cells expressing the transcription factor Neurogenin-3 (Ngn3). Progenitor cells are more differentiated than multipotent stem cells and can differentiate into only few cell types. In particular, Ngn3-positive endocrine progenitor cells have the ability to differentiate into the five pancreatic endocrine cell types (a, β, 6, E and PP). The expression of Ngn3 may be assessed by any method known by the skilled person such as immunochemistry using an anti-Ngn3 antibody or quantitative RT-PCR.

The terms “NeuroD” and “NeuroD1” are used interchangeably and identify a protein expressed in pancreatic endocrine progenitor cells and the gene encoding it.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. The term “selectable marker” as used herein can refer to a gene or to an expression product of the gene, e.g., an encoded protein. In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The term “epigenetics” refers to heritable changes in gene function that do not involve changes in the DNA sequence. Epigenetics most often denotes changes in a chromosome that affect gene activity and expression, but can also be used to describe any heritable phenotypic change that does not derive from a modification of the genome. Such effects on cellular and physiological phenotypic traits can result from external or environmental factors, or be part of normal developmental program. Epigenetics can also refer to functionally relevant changes to the genome that do not involve a change in the nucleotide sequence. Examples of mechanisms that produce such changes are DNA methylation and histone modification, each of which alters how genes are expressed without altering the underlying DNA sequence. Gene expression can be controlled through the action of repressor proteins that attach to silencer regions of the DNA. These epigenetic changes can last through cell divisions for the duration of the cell's life, and can also last for multiple generations even though they do not involve changes in the underlying DNA sequence of the organism. One example of an epigenetic change in eukaryotic biology is the process of cellular differentiation. During morphogenesis, totipotent stem cells become the various pluripotent cells, which in turn can become fully differentiated cells.

The term “epigenetic modifying compound” refers to a chemical compound that can make epigenetic changes genes, i.e., change gene expression(s) without changing DNA sequences. Epigenetic changes can help determine whether genes are turned on or off and can influence the production of proteins in certain cells, e.g., beta-cells. Epigenetic modifications, such as DNA methylation and histone modification, alter DNA accessibility and chromatin structure, thereby regulating patterns of gene expression. These processes are crucial to normal development and differentiation of distinct cell lineages in the adult organism. They can be modified by exogenous influences, and, as such, can contribute to or be the result of environmental alterations of phenotype or pathophenotype. Importantly, epigenetic modification has a crucial role in the regulation of pluripotency genes, which become inactivated during differentiation. Non-limiting exemplary epigenetic modifying compound include a DNA methylation inhibitor, a histone acetyltransferase inhibitor, a histone deacetylase inhibitor, a histone methyltransferase inhibitor, a bromodomain inhibitor, or any combination thereof.

The term “differentiated cell” or its grammatical equivalents is meant any primary cell that is not, in its native form, pluripotent as that term is defined herein. Stated another way, the term “differentiated cell” can refer to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ^{˜}98% of the cells become exocrine, ductular, or matrix cells, and ˜2% become endocrine cells. Early endocrine cells are islet progenitors, which can then differentiate further into insulin-producing cells (e.g. functional endocrine cells) which secrete insulin, glucagon, somatostatin, or pancreatic polypeptide. Endoderm cells can also be differentiate into other cells of endodermal origin, e.g. lung, liver, intestine, thymus etc.

As used herein, the term “somatic cell” can refer to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for converting at least one insulin-positive endocrine cell or precursor thereof to an insulin-producing, glucose responsive cell can be performed both in vivo and in vitro (where in vivo is practiced when at least one insulin-positive endocrine cell or precursor thereof are present within a subject, and where in vitro is practiced using an isolated at least one insulin-positive endocrine cell or precursor thereof maintained in culture).

As used herein, the term “adult cell” can refer to a cell found throughout the body after embryonic development.

The term “endoderm cell” as used herein can refer to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of the respiratory and digestive tracts (e.g. the intestine), the liver and the pancreas.

The term “a cell of endoderm origin” as used herein can refer to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) are develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates. Interest in the development and regeneration of the organs has been fueled by the intense need for hepatocytes and pancreatic β cells in the therapeutic treatment of liver failure and type I diabetes. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

The term “definitive endoderm” as used herein can refer to a cell differentiated from an endoderm cell and which can be differentiated into a SC-β cell (e.g., a pancreatic β cell). A definitive endoderm cell expresses the marker Sox17. Other markers characteristic of definitive endoderm cells include, but are not limited to MIXL2, GATA4, HNF3b, GSC, FGF17, VWF, CALCR, FOXQ1, CXCR4, Cerberus, OTX2, goosecoid, C-Kit, CD99, CMKOR1 and CRIP1. In particular, definitive endoderm cells herein express Sox17 and in some embodiments Sox17 and HNF3B, and do not express significant levels of GATA4, SPARC, APF or DAB. Definitive endoderm cells are not positive for the marker Pdx1 (e.g. they are Pdx1-negative). Definitive endoderm cells have the capacity to differentiate into cells including those of the liver, lung, pancreas, thymus, intestine, stomach and thyroid. The expression of Sox17 and other markers of definitive endoderm may be assessed by any method known by the skilled person such as immunochemistry, e.g., using an anti-Sox17 antibody, or quantitative RT-PCR.

The term “pancreatic endoderm” can refer to a cell of endoderm origin which is capable of differentiating into multiple pancreatic lineages, including pancreatic β cells, but no longer has the capacity to differentiate into non-pancreatic lineages.

The term “primitive gut tube cell” or “gut tube cell” as used herein can refer to a cell differentiated from an endoderm cell and which can be differentiated into a SC-β cell (e.g., a pancreatic β cell). A primitive gut tube cell expresses at least one of the following markers: HNP1-β, HNF3-β or HNF4-α. Primitive gut tube cells have the capacity to differentiate into cells including those of the lung, liver, pancreas, stomach, and intestine. The expression of HNF1-β and other markers of primitive gut tube may be assessed by any method known by the skilled person such as immunochemistry, e.g., using an anti-HNF1-β antibody.

The term “stem cell” as used herein, can refer to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” can refer to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retro-differentiation” by persons of ordinary skill in the art. As used herein, the term “pluripotent stem cell” includes embryonic stem cells, induced pluripotent stem cells, placental stem cells, etc.

The term “pluripotent” as used herein can refer to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and can refer to a pluripotent stem cell artificially derived (e.g., induced or by complete reversal) from a non-pluripotent cell, typically an adult somatic cell, for example, by inducing a forced expression of one or more genes.

The term “phenotype” can refer to one or a number of total biological characteristics that define the cell or organism under a particular set of environmental conditions and factors, regardless of the actual genotype.

The terms “subject,” “patient,” or “individual” are used interchangeably herein, and can refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject can refer to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g, dog, cat, horse, and the like, or production mammal, e.g. cow, sheep, pig, and the like. “Patient in need thereof” or “subject in need thereof” is referred to herein as a patient diagnosed with or suspected of having a disease or disorder, for instance, but not restricted to diabetes.

“Administering” used herein can refer to providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise the cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The terms “treat,” “treating,” “treatment,” and their grammatical equivalents, as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” can refer to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “therapeutically effective amount”, therapeutic amount”, or its grammatical equivalents can refer to an amount effective, at dosages and for periods of time necessary, to achieve a desired therapeutic result. The therapeutically effective amount can vary according to factors such as the disease state, age, sex, and weight of the individual and the ability of a composition described herein to elicit a desired response in one or more subjects. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject of can be “prophylactic,” e.g., the effect completely or partially prevents a disease or symptom thereof. A “prophylactically effective amount” can refer to an amount effective, at dosages and for periods of time necessary, to achieve a desired prophylactic result (e.g., prevention of disease onset).

Some numerical values disclosed throughout are referred to as, for example, “X is at least or at least about 100; or 200 [or any numerical number].” This numerical value includes the number itself and all of the following:
a. X is at least 100;
b. X is at least 200;
c. X is at least about 100; and
d. X is at least about 200.

All these different combinations are contemplated by the numerical values disclosed throughout. All disclosed numerical values should be interpreted in this manner, whether it refers to an administration of a therapeutic agent or referring to days, months, years, weight, dosage amounts, etc., unless otherwise specifically indicated to the contrary.

The ranges disclosed throughout are sometimes referred to as, for example, “X is administered on or on about day 1 to 2; or 2 to 3 [or any numerical range].” This range includes the numbers themselves (e.g., the endpoints of the range) and all of the following:
i) X being administered on between day 1 and day 2;
ii) X being administered on between day 2 and day 3;
iii) X being administered on between about day 1 and day 2;
iv) X being administered on between about day 2 and day 3;
v) X being administered on between day 1 and about day 2;
vi) X being administered on between day 2 and about day 3;
vii) X being administered on between about day 1 and about day 2; and
viii) X being administered on between about day 2 and about day 3.

All these different combinations are contemplated by the ranges disclosed throughout. All disclosed ranges should be interpreted in this manner, whether it refers to an administration of a therapeutic agent or referring to days, months, years, weight, dosage amounts, etc., unless otherwise specifically indicated to the contrary.

### Stages of Differentiation

In some embodiments, pancreatic differentiation as disclosed herein is carried out in a step-wise manner. In the step-wise progression, “Stage 1” or “S1” refers to the first step in the differentiation process, the differentiation of pluripotent stem cells into cells expressing markers characteristic of definitive endoderm cells (“DE”, “Stage 1 cells” or “S1 cells”). “Stage 2” refers to the second step, the differentiation of cells expressing markers characteristic of definitive endoderm cells into cells expressing markers characteristic of gut tube cells (“GT”, “Stage 2 cells” or “S2 cells”). “Stage 3” refers to the third step, the differentiation of cells expressing markers characteristic of gut tube cells into cells expressing markers characteristic of pancreatic progenitor 1 cells (“PP1”, “Stage 3 cells” or “S3 cells”). “Stage 4” refers to the fourth step, the differentiation of cells expressing markers characteristic of pancreatic progenitor 1 cells into cells expressing markers characteristic of pancreatic progenitor 2 cells (“PP2”, “Stage 4 cells” or “S4 cells”). “Stage 5” refers to the fifth step, the differentiation of cells expressing markers characteristic of pancreatic progenitor 2 cells into cells expressing markers characteristic of pancreatic endoderm cells and/or pancreatic endocrine progenitor cells (“EN”, “Stage 5 cells” or “S5 cells”). “Stage 6” refers to the differentiation of cells expressing markers characteristic of pancreatic endocrine progenitor cells into cells expressing markers characteristic of pancreatic endocrine β cells (“SC-β cells”) or pancreatic endocrine α cells (“SC-α cells”). It should be appreciated, however, that not all cells in a particular population progress through these stages at the same rate, i.e., some cells may have progressed less, or more, down the differentiation pathway than the majority of cells present in the population.

### Culture Medium and Agents

### TGF-β Signaling Pathway Inhibitor

Exemplary TGF-β signaling pathway inhibitors include, without limitation, ALK5 inhibitor II (CAS 446859-33-2, an ATP-competitive inhibitor of TGF-B Ri kinase, also known as RepSox, IIJPAC Name: 2-[5-(6-methylpyridin-2-yl)-1H-pyrazol-4-yl]-1,5-naphthyridine, an analog or derivative of ALK5 inhibitor II, such as an analog or derivative of ALK5 inhibitor II described in U.S. Pub. No. 2012/0021519, a TGF-β receptor inhibitor described in U.S. Pub. No. 2010/0267731, an ALK5 inhibitor described in U.S. Pub Nos. 2009/0186076 and 2007/0142376, including e.g., A83-01, 431542, D4476, GW788388, LY364947, LY580276, SB525334, SB505124, SD208, GW6604, or GW788388.

In some embodiments, the TGF-3 signaling pathway inhibitor can have the following structure:

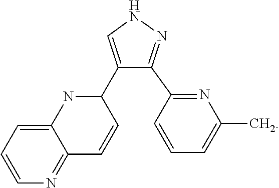

In some embodiments, the concentration of the TGF-β signaling pathway inhibitor can be from about 0.1-110 μM, 0.1-50 μM, 0.1-25 μM, or 0.1-10 μM. In some embodiments, the concentration of the TGF-β signaling pathway inhibitor can be about 10 μM. In some embodiments, the TGF-β signaling pathway inhibitor is an Alk5 inhibitor II and concentration of the inhibitor is about 10 μM.

### Thyroid Hormone Signaling Pathway Activator

Exemplary thyroid hormone signaling pathway activators include, without limitation, triiodothyronine (T3), an analog or derivative of T3, for example, selective and non-selective thyromimetics, TRJ selective agonist-GC-1, GC-24,4-Hydroxy-PCB 106, MB0781 1, MB07344,3,5-diiodothyropropionic acid (DITPA); the selective TR-β agonist GC-1; 3-Iodothyronamine (T(1)AM) and 3,3′,5-triiodothyroacetic acid (Triac) (bioactive metabolites of the hormone thyroxine (T(4)); KB-21 15 and KB-141; thyronamines; SKF L-94901; DIBIT; 3′-AC-T2; tetraiodothyroacetic acid (Tetrac) and triiodothyroacetic acid (Triac) (via oxidative deamination and decarboxylation of thyroxine [T4] and triiodothyronine [T3]alanine chain), 3,3′,5′-triiodothyronine (rT3) (via T4 and T3 deiodination), 3,3′-diiodothyronine (3,3′-T2) and 3,5-diiodothyronine (T2) (via T4, T3, and rT3 deiodination), and 3-iodothyronamine (T1AM) and thyronamine (TOAM) (via T4 and T3 deiodination and amino acid decarboxylation), as well as for TH structural analogs, such as 3,5,3′-triiodothyropropionic acid (Triprop), 3,5-dibromo-3-pyridazinone-1-thyronine (L-940901), N-[3,5-dimethyl-4-(4′-hydroxy-3^{f}-isopropylphenoxy)-phenyl]-oxamic acid (CGS 23425), 3,5-dimethyl-4-[(4′-hydroxy-3′-isopropylbenzyl)-phenoxy]acetic acid (GC-1), 3,5-dichloro-4-[(4-hydroxy-3-isopropylphenoxy)phenyl]acetic acid (KB-141), and 3,5-diiodothyropropionic acid (DITPA).

In some embodiments, the thyroid hormone signaling pathway activator can comprise a prodrug or prohormone of T3, such as T4 thyroid hormone (e.g., thyroxine or L-3,5,3′,5′-tetraiodothyronine). In some embodiments, the thyroid hormone signaling pathway activator can be an iodothyronine composition described in U.S. Pat. No. 7,163,918. In some embodiments, the thyroid hormone signaling pathway activator can be 2-[4-[[4-Hydroxy-3-(1-methylethyl)phenyl]methyl]-3,5-dimethylphenoxy]acetic acid (GC-1). GC-1 is a thyromimetic, high affinity agonist at thyroid hormone receptor (TR) β and TRoc receptors (K_{D} values are 67 and 440 p respectively). GC-1 displays 5- and 100-fold greater potency than the endogenous agonist T3 in vitro at TRoti and TR i receptors respectively.

In some embodiments, the thyroid hormone signaling pathway activators can have the following structure:

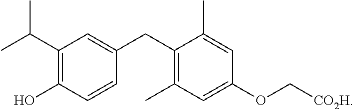 [SMILES: Cc1cc(OCC(=O)O)cc(C)c1Cc1ccc(O)c(C(C)C)c1]

In some embodiments, the concentration of the thyroid hormone signaling pathway activator can be from about 0.1-110 M, 0.1-50 μM, 0.1-25 μM, or 0.1-10 μM. In some embodiments, the concentration of the thyroid hormone signaling pathway activator can be about 1 μM. In some embodiments, the thyroid hormone signaling pathway activator is GC-1 and the concentration of the activator is about 1 μM.

### Protein Kinase Inhibitor

Exemplary protein kinase inhibitors include, without limitation, staurosporine, an analog of staurosporine, such as Ro-31-8220, a bisindolylmaleimide (Bis) compound, 10′-{5″-[(methoxycarbonyl)amino]-2″-methyl}-phenylaminocarbonylstaurosporine, a staralog (see, e.g., Lopez et al., “Staurosporine-derived inhibitors broaden the scope of analog-sensitive kinase technology”, J. Am. Chem. Soc. 2013; i 35(48): 1 8153-18159), and cgp4125 1. In some embodiments, the protein kinase inhibitor can be staurosporine.

In some embodiments, the concentration of the protein kinase inhibitor can be from about 0.1-110 nM, 0.1-50 nM, 0.1-25 nM, 0.1-10 nM, or 0.1-5 nM. In some embodiments, the concentration of the protein kinase inhibitor can be about 3 nM. In some embodiments, the protein kinase inhibitor is staurosporine (SSP) and the concentration of the activator is about 3 nM.

In some embodiments, the protein kinase inhibitor can have the following structure:

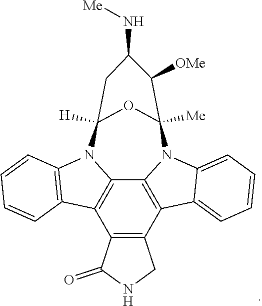 [SMILES: CN[C@@H]1C[C@H]2O[C@@](C)([C@@H]1OC)n1c3ccccc3c3c4c(c5c6ccccc6n2c5c31)C(=O)NC4]

### Bone Morphogenic Protein (BMP) Signaling Pathway Inhibitor

Exemplary BMP signaling pathway inhibitors include, without limitation, 4-[6-(4-piperazin-1-ylphenyl)pyrazolo [1,5-a]pyrimidin-3-yl]quinolone (LDN 193 189; also known as LDN1931 89, 1062368-24-4, LDN-193189, DM 3189, DM-3189, and referred to herein as LDN), an analog or derivative of LDN193189, e.g., a salt (e.g., LDN193189 hydrochloride), hydrate, solvent, ester, or prodrug of LDN193189, or a compound of Formula I from U.S. Patent Publication No. 2011/0053930. In accordance with aspects of the present invention, the BMP signaling pathway inhibitor comprises LDN193189.

In some embodiments, the BMP signaling pathway inhibitor can have the following structure:

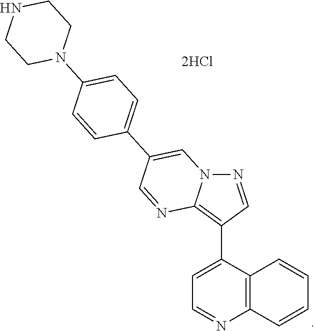 [SMILES: c1ccc2c(-c3cnn4cc(-c5ccc(N6CCNCC6)cc5)cnc34)ccnc2c1]

In some embodiments, the concentration of the BMP signaling pathway inhibitor can be from about 0.1-110 nM, 0.1-100 nM, or 0.1-50 nM. In some embodiments, the concentration of the BMP signaling pathway inhibitor can be about 100 nM. In some embodiments, the protein BMP signaling pathway inhibitor is LDN193189 and the concentration of the activator is about 100 nM.

### Rho-Associated Protein Kinase (ROCK) Inhibitor

Exemplary ROCK inhibitors include, but are not limited to a small organic molecule ROCK inhibitor selected from the group consisting of N-[(15)-2-Hydroxy-1-phenylethyl]-iV-[4-(4-pyridinyl)phenyl]-urea (AS 1892802), fasudil hydrochloride (also known as HA 1077), -[3-[[2-(4-Amino-J,2,5-oxadiazol-3-yl)-1-ethyl-1H-imidazo[4,5-c]pyridin-6-yl]oxy]phenyl]-4-[2-(4-morpholinyl)ethoxy]benzamide (GS 269962), 4-[4-(Trifluoromethyl)phenyl]-N-(6-Fluoro-1H-indazol-5-yl)-2-methyl-6-oxo-1,4,5,6-tetrahydro-3-pyridinecarboxamide (GSK 429286), (5)-(+)-2-Methyl-1-[(4-methyl-5-isoquinolinyl)sulfonyl]-hexahydro-1H-1,4-diazepine dihydrochloride (H1 152 dihydrochloride), (5)-(+)-4-Glycyl-2-methyl-1-[(4-methyl-5-isoquinolinyl)sulfonyl]-hexahydro-1H-1,4-diazepine dihydrochloride (glycyl-M 1 152 dihydrochloride), N-[(3-Hydroxyphenyl)methyl]-V-[4-(4-pyridinyl)-2-thiazolyl]urea dihydrochloride (RKI 1447 dihydrochloride), (35)-1-[[2-(4-Amino-1,2,5-oxadiazol-3-yl)-1-ethyl-1H-imidazo[4,5-c]pyridin-7-yl]carbonyl]-3-pyrrolidinamine dihydrochloride (SB772077B dihydrochloride), N-[2-[2-(Dimethylamino)ethoxyJ-4-(1H-pyrazol-4-yl)phenyl-2,3-dihydro-1,4-benzodioxin-2-carboxamide dihydrochloride (SR 3677 dihydrochloride), and tra«5′-4-[(1/?)-1-Aminoethyl]-N-4-pyridinylcyclohexanecarboxamide dihydrochloride (Y-27632 dihydrochloride), N-Benzyl-[2-(pyrimidin-4-yl)amino]thiazole-4-carboxamide (Thiazovivin), Rock Inhibitor, a isoquinolinesulfonamide compound (Rho Kinase Inhibitor), N-(4-Pyridyl)-N′-(2,4,6-trichlorophenyl)urea (Rho Kinase Inhibitor II), 3-(4-Pyridyl)-1H-indole (Rho Kinase Inhibitor III, Rockout), and 4-pyrazoleboronic acid pinacol ester; a Rock antibody commercially available from Santa Cruz Biotechnology selected from the group consisting of Rock-1 (B 1), Rock-1 (C-19), Rock-1 (H-11), Rock-1 (G-6), Rock-1 (H-85), Rock-1 (K-18), Rock-2 (C-20), Rock-2 (D-2), Rock-2 (D-11), Rock-2 (N-19), Rock-2 (H-85), Rock-2 (30-J); a ROCK CRISPR/Cas9 knockout plasmid selected from the group consisting of Rock-1 CRISPR/Cas9 KO plasmid (h), Rock-2 CRISPR/Cas9 KO plasmid (h), Rock-1 CR1SPR/Cas9 KO plasmid (m), Rock-2 CRISPR/Cas9 KO plasmid (m); a ROCK siRNA, shRNA plasmid and/or shRNA lentiviral particle gene silencer selected from the group consisting of Rock-1 siRNA (h): sc-29473, Rock-1 siRNA (m): sc-36432, Rock-1 siRNA (r): sc-72179, Rock-2 siRNA (h): sc-29474, Rock-2 siRNA (m): sc-36433, Rock-2 siRNA (r): sc-108088.

In some embodiments, the ROCK inhibitor comprises Y-27632. In some embodiments, the ROCK inhibitor is thiazovivin.

In some embodiments, the ROCK inhibitor can have the following structure:

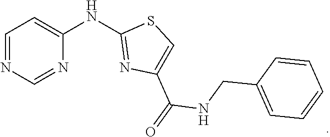 [SMILES: O=C(NCc1ccccc1)c1csc(Nc2ccncn2)n1]

In some embodiments, the concentration of the ROCK inhibitor can be from about 0.1-110 μM, 0.1-50 μM, 0.1-25 μM, or 0.1-10 μM. In some embodiments, the concentration of the ROCK inhibitor can be about 2.5 μM. In some embodiments, the ROCK inhibitor is thiazovivin and the concentration of the inhibitor is about 2.5 μM.

In some embodiments, the concentration of the ROCK inhibitor (e.g., Y-27632 or Thiazovivin), can be about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM.

### Histone Methyltransferase Inhibitors

In some embodiments, a histone methyltransferase inhibitor may be used as an epigenetic modifier. Exemplary histone methyltransferase inhibitors can include, but are not limited to, e.g., 3-Deazaneplanocin A hydrochloride (DZNep-(1S,2R,5R)-5-(4-amino-1H-imidazo[4,5-c]pyridin-1-yl)-3-(hydroxymethyl)cyclopent-3-ene-1,2-diol); Bix-01294, UNC0638, BRDD4770, EPZ004777, AZ505, PDB4e47, alproic acid, vorinostat, romidepsin, entinostat abexinostat, givinostat, and mocetinostat, butyrate, a serine protease inhibitor (serpin) family member. In some embodiments, the histone methyltransferase inhibitor can be DZNep. In some embodiments, the histone methyltransferase inhibitor can have the following structure:

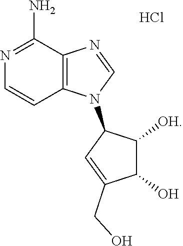 [SMILES: Nc1nccc2c1ncn2[C@@H]1C=C(CO)[C@@H](O)[C@H]1O ; Cl]

In some embodiments, the concentration of the histone methyltransferase inhibitor can be from about 0.1-110 nM, 0.1-100 nM, or 0.1-50 nM. In some embodiments, the concentration of the histone methyltransferase inhibitor can be about 100 nM. In some embodiments, the histone methyltransferase inhibitor is DZNep and the concentration of the inhibitor is about 100 nM.

In some embodiments, the concentration of the histone methyltransferase inhibitor can be about 0.01 μM, about 0.025 μM, about 0.05 μM, about 0.075 μM, about 0.1 μM, about 0.15 μM, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 15 μM, about 20 μM, about 25 μM, about 30 μM, about 35 μM, about 40 μM, about 50 μM, or about 100 μM.

### MGLL Inhibitors

Exemplary MGLL (Monoglyceride Lipase) inhibitors include, but are not limited to, e.g., JJKK048, KML29, NF1819, JW642, JZL184, JZL195, JZP361, pristimerin, or URB602.

In some embodiments, the MGLL inhibitor can be JJKK048. In some embodiments, the MGLL inhibitor can be KML29. In some embodiments, the MGLL inhibitor can be NF1819.

In some embodiments, the MGLL inhibitor can have the following structure:

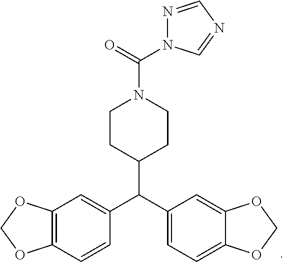 [SMILES: O=C(N1CCC(C(c2ccc3c(c2)OCO3)c2ccc3c(c2)OCO3)CC1)n1cncn1]

In some embodiments the MGLL inhibitor can have the following structure:

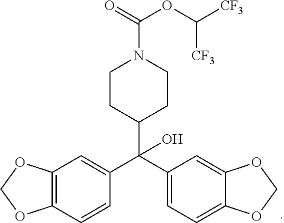 [SMILES: O=C(OC(C(F)(F)F)C(F)(F)F)N1CCC(C(O)(c2ccc3c(c2)OCO3)c2ccc3c(c2)OCO3)CC1]

In some embodiments the MGLL inhibitor can have the following structure:

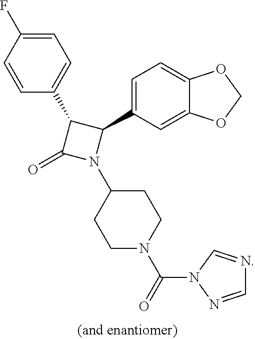 [SMILES: O=C1[C@H](c2ccc(F)cc2)[C@@H](c2ccc3c(c2)OCO3)N1C1CCN(C(=O)n2cncn2)CC1]

In some embodiments, the concentration of the MGLL inhibitor is from about 0.1 μM-100 μM. In some embodiments, the concentration of the MGLL inhibitor is about 0.1 μM, 1 μM, 10 μM, 20 PM, 30 μM, 40 μM, 50 μM, 60 μM, 70 μM, 80 μM, 90 μM, 100 μM.

In some embodiments, the MGLL inhibitor is JJKK048 and the concentration is from about 0.1 μM-100 μM. In some embodiments, the MGLL inhibitor is KML29 and the concentration is from about 0.1 μM-100 μM. In some embodiments, the MGLL inhibitor is NF1819 and the concentration is from about 0.1 μM-100 μM.

In some embodiments, the MGLL inhibitor is JJKK048 and the concentration is 1 μM. In some embodiments, the MGLL inhibitor is KML29 and the concentration is 10 μM. In some embodiments, the MGLL inhibitor is NF1819 and the concentration is 10 μM.

### Lipids

Exemplary lipids include, but are not limited to, fatty acids, e.g., a saturated fatty acid or a unsaturated fatty acid. In some embodiments, the lipid is a saturated fatty acid. In some embodiments, the lipid is a unsaturated fatty acid. Exemplary saturated fatty acids include, but are not limited to, e.g., palmitate, palmitic acid, butyric acid, valeric acid, caproic acid, enanthic acid, caprylic acid, pelargonic acid, capric acid, undecylic acid, lauric acid, tridecylic acid, myristic acid, pentadecylic acid, margaric acid, Stearic acid, Nonadecylic acid, Arachidic acid, Heneicosylic acid, Behenic acid, Tricosylic acid, Lignoceric acid, Pentacosylic acid, Cerotic acid, Heptacosylic acid, Montanic acid, Nonacosylic acid, Melissic acid, Hentriacontylic acid, Lacceroic acid, Psyllic acid, Geddic acid, Ceroplastic acid, Hexatriacontylic acid, Heptatriacontanoic acid, Octatriacontanoic acid, Nonatriacontanoic acid, or Tetracontanoic acid; or a salt or ester thereof.

In some embodiments, the saturated fatty acid is palmitic acid, or a salt or ester thereof. In some embodiments, the saturated fatty acid is palmitate.

Exemplary unsaturated fatty acids include, but are not limited to, e.g., oleic acid, linoleic acid, palmitoleic acid, stearidonic acid, eicosapentaenoic acid, docosahexaenoic acid, linolelaidic acid, γ-Linolenic acid, dihomo-γ-linolenic acid, arachidonic acid, docosatetraenoic acid, vaccenic acid, paullinic acid, elaidic acid, gondoic acid, erucic acid, nervonic acid, or mead acid, or a salt or ester thereof.

In some embodiments, the unsaturated fatty acid is oleic acid. In some embodiments, the unsaturated fatty acid is linoleic acid. In some embodiments, the unsaturated fatty acid is palmitoleic acid.

### Reprogramming

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

As used herein, the term “reprogramming factor” is intended to refer to a molecule that is associated with cell “reprogramming”, that is, differentiation, and/or de-differentiation, and/or transdifferentiation, such that a cell converts to a different cell type or phenotype. Reprogramming factors generally affect expression of genes associated with cell differentiation, de-differentiation and/or transdifferentiation. Transcription factors are examples of reprogramming factors.

The term “differentiation” and their grammatical equivalents as used herein refers to the process by which a less specialized cell (i.e., a more naive cell with a higher cell potency) becomes a more specialized cell type (i.e., a less naive cell with a lower cell potency); and that the term “de-differentiation” refers to the process by which a more specialized cell becomes a less specialized cell type (i.e., a more naive cell with a higher cell potency); and that the term “transdifferentiation” refers to the process by which a cell of a particular cell type converts to another cell type without significantly changing its “cell potency” or “naivety” level. Without wishing to be bound by theory, it is thought that cells “transdifferentiate” when they convert from one lineage-committed cell type or terminally differentiated cell type to another lineage-committed cell type or terminally differentiated cell type, without significantly changing their “cell potency” or “naivety” level.

As used herein, the term “cell potency” is to be understood as referring to the ability of a cell to differentiate into cells of different lineages. For example, a pluripotent cell (e.g., a stem cell) has the potential to differentiate into cells of any of the three germ layers, that is, endoderm (interior stomach lining, gastrointestinal tract, the lungs), mesoderm (muscle, bone, blood, urogenital), or ectoderm (epidermal tissues and nervous system), and accordingly has high cell potency; a multipotent cell (e.g., a stem cell or an induced stem cell of a certain type) has the ability to give rise to cells from a multiple, but limited, number of lineages (such as hematopoietic stem cells, cardiac stem cells, or neural stem cells, etc) comparatively has a lower cell potency than pluripotent cells. Cells that are committed to a particular lineage or are terminally differentiated can have yet a lower cell potency. Specific examples of transdifferentiation known in the art include the conversion of e.g., fibroblasts beta cells or from pancreatic exocrine cells to beta cells etc.

Accordingly, the cell may be caused to differentiate into a more naive cell (e.g., a terminally differentiated cell may be differentiated to be multipotent or pluripotent); or the cell may be caused to de-differentiate into a less naive cell (e.g., a multipotent or pluripotent cell can be differentiated into a lineage-committed cell or a terminally differentiated cell). However, in an embodiment, the cell may be caused to convert or transdifferentiate from one cell type (or phenotype) to another cell type (or phenotype), for example, with a similar cell potency level. Accordingly, in an embodiment of the present disclosure, the inducing steps of the present disclosure can reprogram the cells of the present disclosure to differentiate, de-differentiate and/or transdifferentiate. In an embodiment of the present disclosure, the inducing steps of the present disclosure may reprogram the cells to transdifferentiate.

Methods of reprogramming or inducing a particular type of cell to become another type of cell, for example, by differentiation, de-differentiation and/or transdifferentiation using one or more exogenous polynucleotide or polypeptide reprogramming factors are known to the person skilled in the art. Such methods may rely on the introduction of genetic material encoding one or more transcription factor(s) or other polypeptide(s) associated with cell reprogramming. For example, Pdx1, Ngn3 and MafA, or functional fragments thereof are all known to encode peptides that can induce cell differentiation, de-differentiation and/or transdifferentiation of the cells of the present disclosure. In some methods known to the person skilled in the art, exogenous polypeptides (e.g. recombinant polypeptides) encoded by reprogramming genes (such as the above genes) are contacted with the cells to induce, for example, cells of the present disclosure. The person skilled in the art will appreciate that other genes may be associated with reprogramming of cells, and exogenous molecules encoding such genes (or functional fragments thereof) and the encoded polypeptides are also considered to be polynucleotide or polypeptide reprogramming factors (e.g. polynucleotides or polypeptides that in turn affect expression levels of another gene associated with cell reprogramming). For example, it has been shown that the introduction of exogenous polynucleotide or polypeptide epigenetic gene silencers that decrease p53 inactivation increase the efficiency of inducing induced pluripotent stem cells (iPSC). Accordingly, exogenous polynucleotides or polypeptides encoding epigenetic silencers and other genes or proteins that may be directly or indirectly involved in cell reprogramming or increasing cell programming efficiency would be considered to constitute an exogenous polynucleotide or polypeptide reprogramming factor. The person skilled in the art will appreciate that other methods of influencing cell reprogramming exist, such as introducing RNAi molecules (or genetic material encoding RNAi molecules) that can knock down expression of genes involved in inhibiting cell reprogramming. Accordingly, any exogenous polynucleotide molecule or polypeptide molecule that is associated with cell reprogramming, or enhances cell reprogramming, is to be understood to be an exogenous polynucleotide or polypeptide reprogramming factor as described herein.

In some embodiments of the present disclosure, the method excludes the use of reprogramming factor(s) that are not small molecules. However, it will be appreciated that the method may utilize “routine” tissue culture components such as culture media, serum, serum substitutes, supplements, antibiotics, etc, such as RPMI, Renal Epithelial Basal Medium (REBM), Dulbecco's Modified Eagle Medium (DMEM), MCDB131 medium, CMRL 1066 medium, F12, fetal calf serum (FCS), foetal bovine serum (FBS), bovine serum albumin (BSA), D-glucose, L-glutamine, GlutaMAX™-1 (dipeptide, L-alanine-L-glutamine), B27, heparin, progesterone, putrescine, laminin, nicotinamide, insulin, transferrin, sodium selenite, selenium, ethanolamine, human epidermal growth factor (hEGF), basic fibroblast growth factor (bFGF), hydrocortisone, epinephrine, normacin, penicillin, streptomycin, gentamicin and amphotericin, etc. It is to be understood that these typical tissue culture components (and other similar tissue culture components that are routinely used in tissue culture) are not small molecule reprogramming molecules for the purposes of the present disclosure. Indeed, these components are either not small molecules as defined herein and/or are not reprogramming factors as defined herein.

Accordingly, in an embodiment, the present disclosure does not involve a culturing step of the cell(s) with one or more exogenous polynucleotide or polypeptide reprogramming factor(s). Accordingly, in an embodiment, the method of the present disclosure does not involve the introduction of one or more exogenous polynucleotide or polypeptide reprogramming factor(s), e.g., by introducing transposons, viral transgenic vectors (such as retroviral vectors), plasmids, mRNA, miRNA, peptides, or fragments of any of these molecules, that are involved in producing induced beta cells or, otherwise, inducing cells of the present disclosure to differentiate, de-differentiation and/or transdifferentiate.

That is, in an embodiment, the method occurs in the absence of one or more exogenous polynucleotide or polypeptide reprogramming factor(s). Accordingly, it is to be understood that in an embodiment, the method of the present disclosure utilizes small molecules to reprogram cells, without the addition of polypeptide transcription factors; other polypeptide factors specifically associated with inducing differentiation, de-differentiation, and/or transdifferentiation; polynucleotide sequences encoding polypeptide transcription factors, polynucleotide sequences encoding other polypeptide factors specifically associated with inducing differentiation, de-differentiation, and/or transdifferentiation; mRNA; interference RNA; microRNA and fragments thereof.

### Stem Cells

The term “stem cell” is used herein to refer to a cell (e.g., plant stem cell, vertebrate stem cell) that has the ability both to self-renew and to generate a differentiated cell type (Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny. In an embodiment, the host cell is an adult stem cell, a somatic stem cell, a non-embryonic stem cell, an embryonic stem cell, hematopoietic stem cell, an include pluripotent stem cells, and a trophoblast stem cell.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs can be in the form of an established cell line, they can be obtained directly from primary embryonic tissue, or they can be derived from a somatic cell.

By “embryonic stem cell” (ESC) is meant a PSC that is isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells can be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806, each of which is incorporated herein by its entirety. Methods for proliferating hESCs in the undifferentiated form are described in WO 99/20741, WO 01/51616, and WO 03/020920, each of which is incorporated herein by its entirety.

By “embryonic germ stem cell” (EGSC) or “embryonic germ cell” or “EG cell”, it is meant a PSC that is derived from germ cells and/or germ cell progenitors, e.g. primordial germ cells, i.e. those that can become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, each of which are incorporated herein by its entirety.

By “induced pluripotent stem cell” or “iPSC”, it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs can be found in, for example, U.S. Patent Publication Nos. US20090047263, US20090068742, US20090191159, US20090227032, US20090246875, and US20090304646, each of which are incorporated herein by its entirety. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell”, it is meant any cell in an organism that, in the absence of experimental manipulation, does not ordinarily give rise to all types of cells in an organism. In other words, somatic cells are cells that have differentiated sufficiently that they do not naturally generate cells of all three germ layers of the body, i.e. ectoderm, mesoderm and endoderm. For example, somatic cells can include both neurons and neural progenitors, the latter of which is able to naturally give rise to all or some cell types of the central nervous system but cannot give rise to cells of the mesoderm or endoderm lineages

In certain examples, the stem cells can be undifferentiated (e.g. a cell not committed to a specific lineage) prior to exposure to at least one β cell maturation factor according to the methods as disclosed herein, whereas in other examples it may be desirable to differentiate the stem cells to one or more intermediate cell types prior to exposure of the at least one cell maturation factor (s) described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of) suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, FISF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature, insulin positive cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al, (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al, (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. In an embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

In some embodiments, the SC-β cell can be derived from one or more of trichocytes, keratinocytes, gonadotropes, corticotropes, thyrotropes, somatotropes, lactotrophs, chromaffin cells, parafollicular cells, glomus cells melanocytes, nevus cells, Merkel cells, odontoblasts, cementoblasts corneal keratocytes, retina Muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, ECL cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells (e.g., PP cells), pancreatic ε cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, splenocytes (e.g., T lymphocytes, B lymphocytes, dendritic cells, microphages, leukocytes), trophoblast stem cells, or any combination thereof.

### Pancreatic Progenitor Cells or Precursors

In some aspects, the present disclosure provides a method of producing a NKX6.1-positive pancreatic progenitor cell from a Pdx1-positive pancreatic progenitor cell comprising contacting a population of cells comprising Pdx1-positive pancreatic progenitor cells or precursors under conditions that promote cell clustering with at least two β cell-maturation factors comprising a) at least one growth factor from the fibroblast growth factor (FGF) family, b) a sonic hedgehog pathway inhibitor, and optionally c) a low concentration of a retinoic acid (RA) signaling pathway activator, for a period of at least five days to induce the differentiation of at least one Pdx1-positive pancreatic progenitor cell in the population into NKX6.1-positive pancreatic progenitor cells, wherein the NKX6.1-positive pancreatic progenitor cells express NKX6.1.

In some embodiments, at least 10% of the Pdx1-positive pancreatic progenitor cells in the population are induced to differentiate into NKX6-1-positive pancreatic progenitor cells. In some embodiments, at least 95% of the Pdx1-positive pancreatic progenitor cells in the population are induced to differentiate into NKX6.1-positive pancreatic progenitor cells. In some embodiments, the NKX6.1-positive pancreatic progenitor cells express Pdx1, NKX6.1, and FoxA2. In some embodiments, the Pdx1-positive pancreatic progenitor cells are produced from a population of pluripotent stem cells selected from the group consisting of embryonic stem cells and induced pluripotent stem cells.

### Stem Cell Derived Beta Cells

In some embodiments, provided herein are methods of using of stem cells to produce SC-beta cells (e.g., mature pancreatic β cells or β-like cells) or precursors thereof. In an embodiment, germ cells may be used in place of, or with, the stem cells to provide at least one SC-β cell, using similar protocols as described in U.S. Patent Application Publication No. US20150240212 and US20150218522, each of which is herein incorporated by reference in its entirety. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

In some embodiments, provided herein are compositions and methods of generating SC-β cells (e.g., pancreatic β cells). Generally, the at least one SC-β cell or precursor thereof, e.g., pancreatic progenitors produced according to the methods disclosed herein can comprise a mixture or combination of different cells, e.g., for example a mixture of cells such as a Pdx1-positive pancreatic progenitors, pancreatic progenitors co-expressing Pdx1 and NKX6.1, a Ngn3-positive endocrine progenitor cell, an insulin-positive endocrine cell (e.g., a β-like cell), and an insulin-positive endocrine cell, and/or other pluripotent or stem cells.

The at least one SC-β cell or precursor thereof can be produced according to any suitable culturing protocol to differentiate a stem cell or pluripotent cell to a desired stage of differentiation. In some embodiments, the at least one SC-β cell or the precursor thereof are produced by culturing at least one pluripotent cell for a period of time and under conditions suitable for the at least one pluripotent cell to differentiate into the at least one SC-β cell or the precursor thereof.

In some embodiments, the at least one SC-β cell or precursor thereof is a substantially pure population of SC-β cells or precursors thereof. In some embodiments, a population of SC-β cells or precursors thereof comprises a mixture of pluripotent cells or differentiated cells. In some embodiments, a population SC-β cells or precursors thereof are substantially free or devoid of embryonic stem cells or pluripotent cells or iPS cells.

In some embodiments, a somatic cell, e.g., fibroblast can be isolated from a subject, for example as a tissue biopsy, such as, for example, a skin biopsy, and reprogrammed into an induced pluripotent stem cell for further differentiation to produce the at least one SC-β cell or precursor thereof for use in the compositions and methods described herein. In some embodiments, a somatic cell, e.g., fibroblast is maintained in culture by methods known by one of ordinary skill in the art, and in some embodiments, propagated prior to being converted into SC-β cells by the methods as disclosed herein.

In some embodiments, the at least one SC-β cell or precursor thereof are maintained in culture by methods known by one of ordinary skill in the art, and in some embodiments, propagated prior to being converted into SC-β cells by the methods as disclosed herein.

Further, at least one SC-β cell or precursor thereof, e.g., pancreatic progenitor can be from any mammalian species, with non-limiting examples including a murine, bovine, simian, porcine, equine, ovine, or human cell. For clarity and simplicity, the description of the methods herein refers to a mammalian at least one SC-β cell or precursor thereof but it should be understood that all of the methods described herein can be readily applied to other cell types of at least one SC-β cell or precursor thereof. In some embodiments, the at least one SC-β cell or precursor thereof is derived from a human individual.

### Cell Clusters of Stem Cell Derived Beta Cells

In some aspects, provided herein are cell clusters that resemble the functions and characteristics of endogenous pancreatic islets. Such cell clusters can mimic the function of endogenous pancreatic islets in regulating metabolism, e.g., glucose metabolism in a subject. Thus, the cell clusters can be transplanted to a subject for treating disease resulting from insufficient pancreatic islet function, e.g., diabetes. The terms “cluster” and “aggregate” can be used interchangeably, and refer to a group of cells that have close cell-to-cell contact, and in some cases, the cells in a cluster can be adhered to one another.

A cell cluster comprises a plurality of cells. In some embodiments, a cell cluster comprises at least 10, at least 50, at least 200, at least 500, at least 750, at least 1000, at least 1500, at least 2000, at least 2500, at least 3000, at least 3500, at least 4000, at least 4500, at least 5000, at least 6000, at least 7000, at least 8000, at least 9000, at least 10,000, at least 20,000, at least 30,000, or at least 50,000 cells. In some embodiments, a cell cluster comprises between 10-10,000 cells, between 50-10,000, between 100-10,000, between 100-10,000, between 1,000-10,000, between 500 and 10,000, between 500 and 5,000, between 500 and 2,500, between 500 and 2,000, between 1,000 and 100,000, between 1,000 and 50,000, between 1,000 and 40,000, between 1,000 and 20,000, between 1,000 and 10,000, between 1,000 and 5,000 and between 1,000 and 3,000 cells. In some embodiments, a cell cluster comprises at least 500 cells. In some embodiments, a cell cluster comprises at least 1,000 cells. In some embodiments, a cell cluster comprises at least 2,000 cells. In some embodiments, a cell cluster comprises at least 5,000 cells. In some embodiments, a cell cluster comprises no more than 100,000, no more than 90,000, no more than 80,000, no more than 70,000, no more than 60,000, no more than 50,000, no more than 40,000, no more than 30,000, no more than 20,000, no more than 10,000, no more than 7,000, no more than 5,000, no more than 3,000, no more than 2,000 cells, or no more than 1,000 cells.

A cell cluster herein can comprise at least one non-native cell, e.g., a non-native pancreatic 3 cell. A non-native cell (e.g., a non-native pancreatic β cell) can share characteristics of an endogenous cell (e.g., an endogenous mature pancreatic β cell), but is different in certain aspects (e.g., gene expression profiles). A non-native cell can be a genetically modified cell. A non-native cell can be a cell differentiated from a progenitor cell, e.g., a stem cell. The stem cell can be an embryonic stem cell (ESC) or induced pluripotent stem cell (iPSC). In some cases, the non-native cell can be a cell differentiated from a progenitor cell in vitro. In some cases, the non-native cell can be a cell differentiated from a progenitor cell in in vivo. For example, a cell cluster can comprise at least one non-native pancreatic 3 cell. The non-native pancreatic β cells can be those described in U.S. patent application Ser. Nos. 14/684,129 and 14/684,101, which are incorporated herein in their entireties. A cell cluster can comprise a plurality of non-native pancreatic β cells. In some cases, at least about 20%, 30%, 40%, 50%, 60%, 70%, 80%, 90%, 99% cells in a cell cluster are non-native pancreatic β cells. A cell cluster can comprise one or more native cells. For example, a cell cluster can comprise one or more primary cells, e.g., primary cells from an endogenous pancreatic islet.

A cell cluster can comprise one or more cells expressing at least one marker of an endogenous cell, e.g., an endogenous mature pancreatic β cell. The term “marker” can refer to a molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein, or a small molecule. In many cases, a marker can refer to a molecule that can be characteristic of a particular type of cell, so that the marker can be called as a marker of the type of cell. For instance, Insulin gene can be referred to as a marker of β cells. In some cases, a marker is a gene. Non-limiting of markers of an endogenous mature pancreatic β cell include insulin, C-peptide, PDX1, NKX6.1, CHGA, MAFA, ZNT8, PAX6, NEUROD1, glucokinase (GCK), SLC2A, PCSK1, KCNJ11, ABCC8, SLC30A8, SNAP25, RAB3A, GAD2, and PTPRN.

A cell cluster can comprise one more cells expressing one or multiple markers of an endogenous cell, e.g., an endogenous mature pancreatic β cell. For example, cell cluster can comprise one or more cells co-expressing at least 2, 3, 4, 5, 6, 7, 8, 9, 10, 15, or 20 marker(s) of an endogenous cell, e.g., an endogenous mature pancreatic β cell. In some cases, a cell cluster comprises cells that express NKX6.1 and C-peptide, both of which can be markers of a β cell.

A cell cluster can comprise a plurality of cells expressing at least one marker of an endogenous cell. For example, at least about 1%, 5%, 10%, 20%, 30%, 40%, 50%, 60%, 70%, 80%, 90%, 95%, 99% cells in a cell cluster can express at least one marker of an endogenous cell. In some cases, all cells in a cell cluster can express a marker of an endogenous cell. In some cases, the endogenous cell can be a pancreatic cell, e.g., a pancreatic β cell, pancreatic α cells, pancreatic β cells, pancreatic Δ cells, or pancreatic γ cells. A cell cluster as provided herein can comprise a heterogeneous group of cells, e.g., cells of different types. For example, the cell cluster can comprises a cell expressing insulin/C-peptide, which can be a marker of a pancreatic β cell, a cell expressing glucagon, which can be a marker of a pancreatic α cell, a cell expressing somatostatin, which can be a marker of a pancreatic Δ cell, a cell expressing pancreatic polypeptides, or any combination thereof.

For example, the cell cluster herein can comprise a plurality of cells expressing one or more markers of an endogenous mature pancreatic β cell. For example, at least about 10%, 20%, 30%, 40%, 50%, 60%, 70%, 80%, 90%, 95%, or 99% cells in the cell cluster can express one or more markers of an endogenous mature pancreatic β cell.

The cell cluster can comprise a plurality of cells expressing CHGA. In some cases, at least about 30%, 40%, 50%, 60%, 70%, 80%, 90%, 95%, or 99% cells in the cell cluster express CHGA. In some cases, at least about 85% cells in a cell cluster can express CHGA. In some cases, a cell cluster can comprise about 90% cell expressing CHGA. In some cases, a cell cluster can comprise about 95% cells expressing CHGA. In certain cases, all cells in a cell cluster can express CHGA.

The cell cluster can comprise a plurality of cells expressing NKX6.1. For example, at least about 30%, 40%, 50%, 60%, 70%, 80%, 90%, 95%, 99% cells in a cell cluster can express NKX6.1. In some cases, at least about 50% cells in a cell cluster can express NKX6.1. In some cases, all cells in a cell cluster can express NKX6.1.

The cell cluster can comprise a plurality of cells expressing C-peptide. For example, at least about 30%, 40%, 50%, 60%, 70%, 80%, 90%, 95%, or 99% cells in a cell cluster can express C-peptide. In some cases, at least about 60% cells in a cell cluster can express C-peptide. In some cases, all cells in a cell cluster can express C-peptide.

The cell cluster can comprise a plurality of cells expressing both NKX6.1 and C-peptide. For example, at least about 10%, 20%, 30%, 40%, 50%, 60%, 70%, 80%, 90%, 95%, or 99% cells in a cell cluster can express C-peptide. In some cases, at least about 35% cells in a cell cluster can express NKX6.1 and C-peptide. In some cases, at least about 40% cells in a cell cluster can express NKX6.1 and C-peptide. In some cases, at least about 35% cells in a cell cluster can express NKX6.1 and C-peptide. In some cases, a cell cluster can comprise about 60% cells expressing NKX6.1 and C-peptide. In some cases, a cell cluster can comprise about 75% cell expressing NKX6.1 and C-peptide. In some cases, all cells in a cell cluster can express NKX6.1 and C-peptide.

The cell cluster can comprise very few to none of stem cells or progenitor cells, e.g., pancreatic progenitor cells. For example, a cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing LIN28. In some examples, a cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing Ki67.

In some cases, a cell cluster can comprise at most 3% cells, at most about 2% cells, at most about 1% cells, at most about 0.5% cells, at most about 0.1% cells, at most about 0.05% cells, at most about 0.01% cells, or no cells expressing SOX2. In some cases, a cell cluster can comprise about 1% cells expressing SOX2. In some cases, a cell cluster can comprise about 0.6% cells expressing SOX2. In some cases, a cell cluster can comprise about 0.3% cells expressing SOX2. In some cases, a cell cluster can comprise about 0.1% cells expressing SOX2.

In some examples, a cell cluster can comprise at most 10% cells, at most about 8% cells, at most about 6% cells, at most about 5% cells, at most about 2% cells, at most about 1% cells, at most about 0.5% cells, at most about 0.1% cells, at most about 0.05% cells, at most about 0.01% cells, or no cells expressing SOX9. In some cases, a cell cluster can comprise about 2% cells expressing SOX9. In some cases, a cell cluster can comprise about 6% cells expressing SOX9. In some cases, a cell cluster can comprise about 1.2% cells expressing SOX9.

A cell cluster herein can exhibit one or multiple glucose stimulated insulin secretion (GSIS) response(s) in vitro when exposed to glucose challenge(s). The GSIS responses can resemble the GSIS responses of an endogenous pancreatic islet. In some cases, the cell cluster exhibits an in vitro GSIS response to a glucose challenge. In some cases, the cell cluster exhibits in vitro GSIS responses to multiple glucose challenges, such as sequential glucose challenges. For example, the cell cluster can exhibit in vitro GSIS responses to at least 2, 3, 4, 5, 6, 7, 8, 9, 10 sequential glucose challenges.

A cell cluster as provided herein can comprise at least one cell exhibiting in vitro GSIS. For example, at least one cell in the cell cluster can be referred to as a mature pancreatic β cell. In some cases, the at least one cell is a non-native pancreatic β cell. In some cases, the at least one cell is a pancreatic β cell resembling a native/endogenous β cell. In some cases, the cell exhibits an in vitro glucose stimulated insulin secretion (GSIS) response. In some cases, the at least one cell exhibits a GSIS response to at least one glucose challenge. In some cases, the cell exhibits a GSIS response to at least two sequential glucose challenges. In some cases, the cell exhibits a GSIS response to at least three sequential glucose challenges

As provided herein, a cell cluster can exhibit GSIS stimulation index similar to an endogenous pancreatic islet. Stimulation index of a cell cluster or a cell can be characterized by the ratio of insulin secreted in response to high glucose concentrations compared to low glucose concentrations. For example, a stimulation index of a cell cluster or a cell as provided herein can be calculated as a ration of insulin secreted in response to 20 mM glucose stimulation versus insulin secreted in response to 2.8 mM glucose stimulation. In some examples, the stimulation index of a cell cluster or a cell as provided herein is greater than or equal to 1, or greater than or equal to 1.1, or greater than or equal to 1.3, or greater than or equal to 2, or greater than or equal to 2.3, or greater than or equal to 2.6. In some instances, the cell cluster or the cell exhibits cytokine-induced apoptosis in response to a cytokine. In some cases, the cytokine comprises interleukin-β (IL-β), interferon-γ (INF-γ), tumor necrosis factor-α (TNF-α), or any combination thereof. In some cases, insulin secretion from the cell cluster or the cell is enhanced in response to an anti-diabetic agent. In some cases, the anti-diabetic agent comprises a secretagogue selected from the group consisting of an incretin mimetic, a sulfonylurea, a meglitinide, and combinations thereof. In some cases, the cell cluster or the cell is monohormonal. In some cases, the cell cluster or the cell exhibits a morphology that resembles the morphology of an endogenous mature pancreatic β cell. In some cases, the cell cluster or the cell exhibits encapsulated crystalline insulin granules under electron microscopy that resemble insulin granules of an endogenous mature pancreatic β cell. In some cases, the cell cluster or the cell exhibits a low rate of replication. In some cases, the cell cluster or the cell exhibits a glucose stimulated Ca²⁺ flux (GSCF) that resembles the GSCF of an endogenous mature pancreatic β cell. In some cases, the cell cluster or the cell exhibits a GSCF response to at least one glucose challenge. In some cases, the cell cluster or the cell exhibits a GSCF response to at least two glucose challenges. In some cases, the cell cluster or the cell exhibits a GSCF response to at least three glucose challenges. In some cases, the cell cluster or the cell exhibits an increased calcium flux. In some cases, the increased calcium flux comprises an increased amount of influx or a ratio of influx at low relative to high glucose concentrations.

A cell cluster as provided herein can exhibit biphasic insulin secretion in response to a high glucose concentration stimulation similar to an endogenous pancreatic islet, e.g., a human pancreatic islet. A biphasic insulin secretion can be a phenomenon characteristic of an endogenous pancreatic islet, e.g., human islet. In some embodiments, response to a high glucose concentration challenge, e.g., 10 mM, 15 mM, 20 mM, or 30 mM, a cell cluster as provided herein, e.g., a reaggregated pancreatic cell cluster, can exhibit a transient increase in insulin secretion to a peak value followed by a rapid decrease to a relatively elevated insulin secretion level, e.g., a level that is higher than an insulin secretion level in response to a lower glucose concentration, e.g., 2.8 mM glucose. Such a transient increase and decrease process can be termed as a first phase of the biphasic insulin secretion pattern. With a persistent high glucose challenge, the first phase can be thus followed by a second phase, in which the insulin secretion by the cell cluster can be maintained at the relatively elevated level. The second phase can last for an extended period, e.g., as long as the high glucose concentration challenge lasts, or relatively longer than the first phase. Such a biphasic insulin secretion pattern can be due to intrinsic cellular signaling changes that are characteristic of a mature native pancreatic β cell.

When transplanted to a subject, a cell cluster can exhibit one or more in vivo GSIS responses when exposed to glucose challenge(s). The cell cluster herein can be capable of exhibiting an in vivo GSIS response within a short period of time after transplanted to a subject. For example, the cell cluster can exhibit an in vivo GSIS within about 6, 12, or 24 hours after transplantation. In some cases, the cell cluster exhibits an in vivo GSIS within about 2 days, 4 days, 6 days, 8 days, 10 days, 12 days, 14 days, 21 days, 28 days, 35 days, or 42 days after transplantation. The amount of insulin secreted by the cell cluster can be similar or higher than an endogenous pancreatic islet. The term “about” in relation to a reference numerical value as used through the application can include a range of values plus or minus 10% from that value. For example, the amount “about 10” includes amounts from 9 to 11. For example, the term “about” in relation to a reference numerical value can also include a range of values plus or minus 10%, 9%, 8%, 7%, 6%, 5%, 4%, 3%, 2%, or 1% from that value.

The cell cluster can maintain the ability of exhibiting in vivo GSIS responses for a period of time after transplanted into a subject. For example, an in vivo GSIS response of the cell cluster can be observed up to at least 2 weeks, 3 weeks, 4 weeks, 5 weeks, 10 weeks, 15 weeks, 20 weeks, 6 months, 7 months, 8 months, 9 months, 10 months, 11 months, 1 year, 2 years, 3 years, 4 years, 5 years, 10 years, 20 years, 30 years, 40 years, 60 years, 80 years, or 100 years after transplantation of the cell cluster into a subject (e.g., a human).

The GSIS of a cell cluster can be measured by a stimulation index. A stimulation index of a cell cluster can equal to the ratio of insulin secreted in response to a high glucose concentration compared to insulin secreted in response to a low glucose concentration. A cell cluster can have a stimulation index similar to an endogenous pancreatic islet. In some cases, a cell cluster has a stimulation index of at least 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

The amount of insulin secreted by a cell cluster in response to a glucose challenge (e.g., a high concentration, such as 20 mM, of glucose) can range from about 0.1 μIU/10³ cells to about 5 μIU/10³ cells, from about 0.2 μIU/10³ cells to about 4 μIU/10³ cells, from about 0.2 μIU/10³ cells to about 3 μIU/10³ cells, or from about 0.23 μIU/10³ cells to about 2.7 μIU/10³ cells. In some cases, the amount of insulin secreted by a cell cluster in response to a glucose challenge (e.g., a high concentration, such as 20 mM, of glucose) is at least 0.05, 0.1, 0.15, 0.2, 0.21, 0.22, 0.23, 0.24, 0.25, 0.26, 0.27, 0.28, 0.29, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μIU/10³ cells.

A cell cluster can secrete both pro-insulin and insulin. For example, a cell cluster can secrete pro-insulin and insulin at a proinsulin-to-insulin ratio substantially the same as the ratio of pro-insulin to insulin secreted by an endogenous pancreatic islet. In some cases, a cell cluster secretes pro-insulin and insulin at a proinsulin-to-insulin ratio of from about 0.01 to about 0.05, from about 0.02 to about 0.04, from about 0.02 to about 0.03, or from 0.029 to about 0.031. In some cases, a cell cluster secretes pro-insulin and insulin at a proinsulin-to-insulin ratio of about 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, or 0.04.

A cell cluster can be in a size similar to an endogenous pancreatic islet. For example, a cell cluster can have a diameter similar to an endogenous pancreatic islet. A diameter of a cell cluster can refer to the largest linear distance between two points on the surface of the cell cluster. In some cases, the diameter of a cell cluster is at most 300 μm, 200 μm, 150 μm, 100 μm, 90 μm, 80 μm, 70 μm, 60 μm, 50 μm, or 40 μm. The diameter of a cell cluster can be from about 75 μm to about 250 μm. The diameter of a cell cluster can be at most 100 μm.

A cell cluster can comprise very few or no dead cells. The cell cluster can be in a size that allows effective diffusion of molecules (e.g., nutrition and gas) from surrounding environment into the core of the cell cluster. The diffused molecule can be important for the survival and function of the cells in the core. In some cases, the cell cluster can have less than about 1%, 2%, 3%, 4%, 5%, 6%, 7%, 8%, 9%, 10% of dead cells, e.g., dead cells in its core. In some cases, a cell cluster can have no dead cell. The dead cells can be apoptotic cells, narcotic cells or any combination thereof.

A cell cluster can comprise one or multiple types of cells. In some cases, a cell cluster comprises one or more types of pancreatic cells. For example, the cell cluster can comprise one or more pancreatic β cell, pancreatic α cells, pancreatic Δ cells, pancreatic γ cells, and any combination thereof. In some cases, the pancreatic cells can be non-native pancreatic cells, e.g., cells derived from stem cells, such as ESCs and/or iPSCs. In some cases, the cell cluster can also comprise one or more progenitor cells of mature pancreatic cells, including iPSCs, ESCs, definitive endoderm cells, primitive gut tube cells, Pdx1-positive pancreatic progenitor cells, Pdx1-positive/NKX6.1-positive pancreatic progenitor cells, Ngn3-positive endocrine progenitor cells, and any combination thereof.

A cell cluster can exhibit cytokine-induced apoptosis in response to cytokines. For example, the cell cluster can exhibit cytokine-induced apoptosis in response to a cytokine such as interleukin-1β (IL-β), interferon-γ (INF-γ), tumor necrosis factor-α (TNF-α), and combinations thereof.

Insulin secretion from a cell cluster herein can be enhanced by an anti-diabetic drug (e.g., an anti-diabetic drug acting on pancreatic β cells ex vivo, in vitro, and/or in vivo). The disclosure can contemplate any known anti-diabetic drug. In some cases, insulin secretion from a cell cluster can be enhanced by a secretagogue. The secretagogue can be an incretin mimetic, a sulfonylurea, a meglitinide, and combinations thereof.

A cell cluster can comprise a monohormonal. For example, the cell cluster can comprise a pancreatic cell (e.g., a pancreatic β cell, pancreatic α cells, pancreatic β cells, pancreatic Δ cells, or pancreatic γ cells) that is monohormonal. In some cases, the cell cluster comprises an insulin-secreting non-native pancreatic cell that is monohormonal. A cell cluster can comprise a polyhormonal. In some case, a cell cluster comprises a monohormonal cell and a polyhormonal cell.

A cell cluster can comprise a cell (e.g., a non-native pancreatic cell) having a morphology that resembles the morphology of an endogenous mature pancreatic β cell. In some cases, the cell cluster can comprise cell encapsulating crystalline insulin granules that resemble insulin granules of an endogenous mature pancreatic β cell, e.g., as detected by electron microscopy. A cell cluster can comprise a plurality cells having a morphology that resembles the morphology of an endogenous mature pancreatic β cell. For example, at least about 10%, 20%, 30%, 40%, 50%, 60%, 70%, 80%, 90% cells in a cell cluster can encapsulate crystalline insulin granules that resemble insulin granules of an endogenous mature pancreatic β cell. In some cases, 100% cells in a cell cluster encapsulate crystalline insulin granules that resemble insulin granules of an endogenous mature pancreatic β cell.

A cell cluster can exhibit glucose-stimulated calcium (Ca²⁺) flux to one or more glucose challenges. In some cases, a cell cluster exhibits a glucose-stimulated Ca²⁺ flux (GSCF) that resembles the GSCF of an endogenous pancreatic islet. In some cases, a cell cluster exhibits a GSCF response to at least 1, 2, 3, 4, 5, 6, 8, or 10 sequential glucose challenges in a manner that resembles the GSCF response of an endogenous pancreatic islet to multiple glucose challenges. A cell cluster can exhibit an in vitro and/or in vivo GSCF response when exposed to a glucose challenge.

A cell cluster can comprise cells originated from any species. For example, a cell cluster can comprise cells from a mammalian species, with non-limiting examples including a murine, bovine, simian, porcine, equine, ovine, or human cell. In some cases, at least one cell in the cell cluster is a human cell.

Provided herein also include compositions comprising a cell clusters disclosed through the application. In addition to the cell cluster, the compositions can further comprise a scaffold or matrix that can be used for transplanting the cell clusters to a subject. A scaffold can provide a structure for the cell cluster to adhere to. The cell cluster can be transplanted to a subject with the scaffold. The scaffold can be biodegradable. In some cases, a scaffold comprises a biodegradable polymer. The biodegradable polymer can be a synthetic polymer, such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates, and biodegradable polyurethanes. The biodegradable polymer can also be a natural polymer, such as albumin, collagen, fibrin, polyamino acids, prolamines, and polysaccharides (e.g., alginate, heparin, and other naturally occurring biodegradable polymers of sugar units). Alternatively, the scaffold can be non-biodegradable. For example, a scaffold can comprise a non-biodegradable polymer, such as polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, and polyethylene oxide.

### Dissociated Cell Compostions and Methods for Making Cell Clusters of Stem Cell Derived Beta Cells

Further disclosed herein are methods for making cell clusters that resemble the function and characteristics of an endogenous tissue or cell cluster, e.g., an endogenous pancreatic islet. The methods can comprise dissociating a first cell cluster and re-aggregating the dissociated cells to a second cell cluster, where the second cell cluster more closely resembles the function and characteristics of an endogenous tissue or cell cluster, e.g., an endogenous pancreatic islet, compared to the first cell cluster. The term “re-aggregating” and its grammatical equivalences as used herein can refer to, when clusters are dissociated into smaller clusters or single cells, the dissociated cells then form new cell-to-cell contacts and form new clusters. The methods can be used for producing a cell cluster in vitro by a) dissociating a plurality of cells from a first cell cluster; and b) culturing the plurality of cells from a) in a medium, thereby allowing the plurality of cells to form a second cell cluster. In some cases, the second cell cluster is an in vitro cell cluster. The first cell cluster can be an in vitro cell cluster, e.g., a cluster formed by a suspension of single cells in vitro in a culture medium. In some cases, the first cell cluster can be an ex vivo cell cluster, e.g., a cell cluster that is formed in a body of a live organism and isolated from said organism. For example, a first cell cluster that the method provided herein is applicable to can be a human pancreatic islet. In some cases, the first cell cluster can be a cadaveric pancreatic islet.

A method provided herein can enrich pancreatic cells in a cell cluster, e.g., a pancreatic β cell, an endocrine cell, or an endocrine progenitor cell. In some examples, the method can reduce or eliminate stem cells or pancreatic progenitor cells from a cell cluster. In some cases, the second cell cluster comprises a higher percentage cells that express chromogranin A as compared the first cell cluster. In some cases, the second cell cluster comprises a higher percentage cells that express NKX6.1 and C-peptide as compared the first cell cluster. In some cases, the second cell cluster comprises a lower percentage cells that express SOX2 as compared the first cell cluster. In some cases, the second in vitro cell cluster comprises a lower percentage of cells that express SOX9 as compared the first cell cluster.

In some cases, the medium comprises a thyroid hormone signaling pathway activator and a transforming growth factor β (TGF-β) signaling pathway inhibitor. In some cases, the medium comprises a) serum, and b) one or both of a thyroid hormone signaling pathway activator and a TGF-β signaling pathway inhibitor. In some cases, the medium for reaggregation as provided herein (reaggregation medium) can comprise no small molecule compounds. For example, the reaggregation medium can comprise no thyroid hormone signaling pathway activator. In some cases, the reaggregation medium does not comprise triiodothyronine (T3), or merely a trace amount of T3. The reaggregation medium can comprise no TGFβ signaling pathway inhibitor. In some cases, the reaggregation medium does not comprise an Alk5 inhibitor (Alk5i), or merely a trace amount of Alk5i.

Dissociating of the first cell cluster can be performed using methods known in the art. Non-limiting exemplary methods for dissociating cell clusters include physical forces (e.g., mechanical dissociation such as cell scraper, trituration through a narrow bore pipette, fine needle aspiration, vortex disaggregation and forced filtration through a fine nylon or stainless steel mesh), enzymatic dissociation using enzymes such as trypsin, collagenase, TrypLE™, and the like, or a combination thereof. After dissociation, cells from the first cell cluster can be in a cell suspension, e.g., a single cell suspension. The term “suspension” as used herein can refer to cell culture conditions in which cells are not attached to a solid support. Cells proliferating in suspension can be stirred while proliferating using apparatus well known to those skilled in the art.

In some embodiments, the disclosure provides for a composition comprising dissociated cells. In some embodiments, the composition does not comprise any cell clusters. In some embodiments, the composition does not comprise any insulin-positive cell clusters. In some embodiments, the composition does not comprise any cell clusters comprising more than 5, 10, 20, 30, 50, 100, 200, 300, 400, 500, 600, 700, 800, 900, or 1000 cells. In some embodiments, the composition does not comprise any cell clusters comprising more than 50 cells. In some embodiments, the composition does not comprise any cell clusters comprising more than 100 cells. In some embodiments, the composition does not comprise any cell clusters comprising more than 500 cells. In some embodiments, the disclosure provides for a composition comprising a plurality of dissociated insulin-positive endocrine progenitor cells. In some embodiments, the dissociated cells are Ngn3-positive. In some embodiments, the dissociated cells are PDX.1 positive. In some embodiments, the dissociated cells are NKX6.1 positive. In some embodiments, the disclosure provides for a composition comprising dissociated cells (e.g., dissociated insulin-positive endocrine progenitor cells) and a BMP signaling pathway inhibitor. In some embodiments, the BMP signaling pathway inhibitor is LDN193189 or a derivative thereof. In some embodiments, the disclosure provides for a composition comprising a plurality of dissociated cells (e.g., dissociated insulin-positive endocrine progenitor cells) and a ROCK inhibitor. In some embodiments, the ROCK inhibitor is thiazovivin, Y-27632, Fasudil/HA1077, or 14-1152, or derivatives thereof. In some embodiments, the disclosure provides for a composition comprising a plurality of dissociated cells (e.g., dissociated insulin-positive endocrine progenitor cells) and a histone methyltransferase inhibitor. In some embodiments, the histone methyltransferase inhibitor is 3-Deazaneplanocin A hydrochloride, or a derivative thereof. In some embodiments, the disclosure provides for a composition comprising a plurality of dissociated cells (e.g., dissociated insulin-positive endocrine progenitor cells) and zinc. In some embodiments, the zinc is in the form of ZnSO₄. In some embodiments, the disclosure provides for a composition comprising a plurality of dissociated cells (e.g., dissociated insulin-positive endocrine progenitor cells) and a monoglyceride lipase (MGLL) inhibitor. In some embodiments, the MGLL inhibitor is JJKK048, KML29, NF1819, JW642, JZL184, JZL195, JZP361, pristimerin, or URB602, or a derivative of any of the foregoing. In some embodiments, the disclosure provides for a composition comprising a plurality of dissociated cells (e.g., dissociated insulin-positive endocrine progenitor cells) and a lipid. In some embodiments, the lipid is a saturated fatty acid. In some embodiments, the saturated fatty acid is palmitate. In some embodiments, the lipid is a unsaturated fatty acid. In some embodiments, the unsaturated fatty acid is oleic acid, linoleic acid, or palmitoleic acid. In some embodiments, the disclosure provides for a composition comprising a plurality of dissociated cells (e.g., dissociated insulin-positive endocrine progenitor cells) and glutamate. In some embodiments, the disclosure provides for a composition comprising a plurality of dissociated cells (e.g., dissociated insulin-positive endocrine progenitor cells) and acetate. In some embodiments, the disclosure provides for a composition comprising a plurality of dissociated cells (e.g., dissociated insulin-positive endocrine progenitor cells) and β-hydroxybutarate. In some embodiments, the disclosure provides for a composition comprising a plurality of dissociated cells (e.g., dissociated insulin-positive endocrine progenitor cells) and L-carnitine. In some embodiments, the disclosure provides for a composition comprising a plurality of dissociated cells (e.g., dissociated insulin-positive endocrine progenitor cells) and taurine. In some embodiments, the disclosure provides for a composition comprising a plurality of dissociated cells (e.g., dissociated insulin-positive endocrine progenitor cells) and formate. In some embodiments, the disclosure provides for a composition comprising a plurality of dissociated cells (e.g., dissociated insulin-positive endocrine progenitor cells) and biotin. In some embodiments, the composition further comprises a serum albumin protein. In some embodiments, the serum albumin protein is a human serum albumin protein. In some embodiments, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some embodiments, less than 90%, less than 85%, les thant 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some embodiments, the composition comprises a TGF-β pathway inhibitor. In some embodiments, the TGF-β pathway inhibitor is Alk5i (SB505124), or a derivative thereof. In some embodiments, the composition comprises a thyroid hormone signaling pathway activator. In some embodiments, the thyroid hormone signaling pathway activator is GC-1 or T3, or a derivative thereof. In some embodiments, the composition comprises a protein kinase inhibitor. In some embodiments, the protein kinase inhibitor is staurosporine. In some embodiments, the composition comprises vitamin C. In particular embodiments, the composition is in vitro. In some embodiments, the composition comprises insulin. In some embodiments, the composition does not comprise a γ secretase inhibitor (e.g., XXI). In some embodiments, the dissociated insulin-positive endocrine progenitor cells were previously frozen.

In some embodiments, the disclosure provides for a composition comprising a plurality of cell clusters. In some embodiments, the disclosure provides for a composition comprising a plurality of cell clusters; wherein the cell clusters comprise insulin-positive cells; wherein at least 30%, at least 35%, at least 40%, at least 45%, at least 50%, at least 55%, at least 60%, or at least 65% of the cells in the composition are viable following 11 days in culture in vitro. In some embodiments, the disclosure provides for a composition comprising a plurality of cell clusters; wherein the cell clusters comprise insulin-positive cells; wherein at at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter. In some embodiments, the disclosure provides for a composition comprising a plurality of cell clusters; wherein the cell clusters comprise insulin-positive cells; wherein at at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, the cell clusters comprise C-peptide positive cells. In some embodiments, the cell clusters comprise somatostatin positive cells. In some embodiments, the cell clusters comprise glucagon positive cells. In some embodiments, at least 30%, at least 35%, at least 40%, at least 45%, at least 50%, at least 55%, at least 60%, or at least 65% of the cells in the composition are viable following 11 days in culture in vitro. In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter. In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, at least 2, 3, 4, 5, 10, 50, 100, 1000, 10000, 100000, or 1000000 cell clusters. In some embodiments, the composition is prepared in accordance with any of the methods disclosed herein. In some embodiments, the disclosure provides for a device comprising the any of the cell compositions disclosed herein. In some embodiments, the disclosure provides for a method of treating a subject with a disease characterized by high blood sugar levels over a prolonged period of time (e.g., diabetes), the method comprising administering any of the compositions disclosed herein or any of the devices disclosed herein to the subject.

In some embodiments, the disclosure provides for a method comprising the step of contacting a plurality of dissociated insulin-positive endocrine progenitor cells with a BMP signaling pathway inhibitor. In some embodiments, the BMP signaling pathway inhibitor is LDN193189 or a derivative thereof. In some embodiments, the disclosure provides for a method comprising the step of contacting a plurality of dissociated insulin-positive endocrine progenitor cells with a ROCK inhibitor. In some embodiments, the ROCK inhibitor is thiazovivin, Y-27632, Fasudil/HA1077, or 14-1152, or derivatives thereof. In some embodiments, the disclosure provides for a method comprising the step of contacting a plurality of dissociated insulin-positive endocrine progenitor cells with a histone methyltransferase inhibitor. In some embodiments, the histone methyltransferase inhibitor is 3-Deazaneplanocin A hydrochloride, or a derivative thereof. In some embodiments, the disclosure provides for a method comprising the step of contacting a plurality of dissociated insulin-positive endocrine progenitor cells with zinc. In some embodiments, the zinc is in the form of ZnSO₄. In some embodiments, the disclosure provides for a method comprising the step of contacting a plurality of dissociated insulin-positive endocrine progenitor cells with a monoglyceride lipase (MGLL) inhibitor. In some embodiments, the MGLL inhibitor is JJKK048, KML29, NF1819, JW642, JZL184, JZL195, JZP361, pristimerin, or URB602, or a derivative of any of the foregoing. In some embodiments, the disclosure provides for a method comprising the step of contacting a plurality of dissociated insulin-positive endocrine progenitor cells with a lipid. In some embodiments, the lipid is a saturated fatty acid. In some embodiments, the saturated fatty acid is palmitate. In some embodiments, the lipid is an unsaturated fatty acid. In some embodiments, the unsaturated fatty acid is oleic acid, linoleic acid, or palmitoleic acid. In some embodiments, the disclosure provides for a method comprising the step of contacting a plurality of dissociated insulin-positive endocrine progenitor cells with glutamate. In some embodiments, the disclosure provides for a method comprising the step of contacting a plurality of dissociated insulin-positive endocrine progenitor cells with acetate. In some embodiments, the disclosure provides for a method comprising the step of contacting a plurality of dissociated insulin-positive endocrine progenitor cells with β-hydroxybutarate. In some embodiments, the disclosure provides for a method comprising the step of contacting a plurality of dissociated insulin-positive endocrine progenitor cells with L-carnitine. In some embodiments, the disclosure provides for a method comprising the step of contacting a plurality of dissociated insulin-positive endocrine progenitor cells with taurine. In some embodiments, the disclosure provides for a method comprising the step of contacting a plurality of dissociated insulin-positive endocrine progenitor cells with formate. In some embodiments, the disclosure provides for a method comprising the step of contacting a plurality of dissociated insulin-positive endocrine progenitor cells with biotin. In some embodiments, the method comprises contacting the plurality of dissociated insulin-positive endocrine progenitor cells with a serum albumin protein. In some embodiments, the serum albumin protein is a human serum albumin protein. In some embodiments, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some embodiments, less than 90%, less than 85%, less than 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some embodiments, the method comprises contacting the plurality of dissociated insulin-positive endocrine progenitor cells with a TGF-β pathway inhibitor. In some embodiments, the TGF-β pathway inhibitor is Alk5i (SB505124), or a derivative thereof. In some embodiments, the method comprises contacting the plurality of dissociated insulin-positive endocrine progenitor cells with a thyroid hormone signaling pathway activator. In some embodiments, the thyroid hormone signaling pathway activator is GC-1 or T3, or a derivative thereof. In some embodiments, the method comprises contacting the plurality of dissociated insulin-positive endocrine progenitor cells with a protein kinase inhibitor. In some embodiments, the protein kinase inhibitor is staurosporine. In some embodiments, the method comprises contacting the plurality of dissociated insulin-positive endocrine progenitor cells with vitamin C. In some embodiments, the method comprises contacting the plurality of dissociated insulin-positive endocrine progenitor cells with insulin. In some embodiments, the method does not comprise the step of contacting the plurality of dissociated insulin-positive endocrine cells with a γ secretase inhibitor (e.g., XXI). In some embodiments, the dissociated insulin-positive endocrine progenitor cells were previously frozen. In some embodiments, the method is performed over the course of 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2- 6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days. In some embodiments, the method results in the reaggregation of the dissociated cells into a plurality of cell clusters. In some embodiments, at least about 40%, 50%, 60%, 70%, 80%, or 90% of the plurality of cell clusters have a diameter from about 50 μm to about 250 μm, from about 75 μm to about 250 μm, or from about 100 μm to about 200 μm. In some embodiments, at least about 40%, 50%, 60%, 65%, 70%, 75%, 80%, 85%, 95%, or 99% of the cells of the plurality of cell clusters of the second cell population are viable. In some embodiments, the method results in the reaggregation of the dissociated cells into at least 2, 3, 4, 5, 10, 50, 100, 1000, 10000, 100000, or 1000000 cell clusters.

In some cases, the method provided herein does not comprise an active cell sorting process, e.g., flow cytometry. In some cases, a cell cluster as described herein can be an unsorted cell cluster. In some cases, a method provided herein does not rely on an active cell sorting for the enrichment or elimination of a particular type of cells in the first cell cluster. In some cases, a method merely requires dissociating the first cell cluster and culturing the plurality of cells dissociated from the first cell cluster in a medium, thereby allowing formation of a second cell cluster.

In some cases, the method provided herein can be applied to dissociate a cell cluster and reaggregate into a new cluster for more than once. For instance, a first cell cluster can be dissociated and reaggregated to form a second cell cluster according to the method provided herein, and the second cell cluster can be further dissociated and reaggregated to form a third cell cluster, and so on. Reaggregation as provided herein can be performed sequentially to a cell cluster for at least 1, 2, 3, 4, 5, 6, 7, 8, 9, or 10 times.

Cell sorting as described herein can refer to a process of isolating a group of cells from a plurality of cells by relying on differences in cell size, shape (morphology), surface protein expression, endogenous signal protein expression, or any combination thereof. In some cases, cell sorting comprises subjecting the cells to flow cytometry. Flow cytometry can be a laser- or impedance-based, biophysical technology. During flow cytometry, one can suspend cells in a stream of fluid and pass them through an electronic detection apparatus. In one type of flow cytometry, fluorescent-activated cell sorting (FACS), based on one or more parameters of the cells' optical properties (e.g., emission wave length upon laser excitation), one can physically separate and thereby purify cells of interest using flow cytometry. As described herein, an unsorted cell cluster can be cell cluster that formed by a plurality of cells that have not been subject to an active cell sorting process, e.g., flow cytometry. An unsorted cell cluster, in some cases referred to as “reaggregated cell cluster,” can be formed by a plurality of cells that are dissociated from an existing cell cluster, and before their reaggregation into the new cell cluster, there can be no active cell sorting process, e.g., flow cytometry or other methods, to isolate one or more particular cell types for the reaggregation as provided herein. In some cases, flow cytometry as discussed herein can be based on one or more signal peptides expressed in the cells. For example, a cell cluster can comprise cells that express a signal peptide (e.g., a fluorescent protein, e.g., green fluorescent protein (GFP) or tdTomato). In some cases, the signal peptide is expressed as an indicator of insulin expression in the cells. For instance, a cell cluster can comprise cell harboring an exogenous nucleic acid sequence coding for GFP under the control of an insulin promoter. The insulin promoter can be an endogenous or exogenous promoter. In some cases, the expression of GFP in these cells can be indicative of insulin expression in said cells. The GFP signal can thus be a marker of a pancreatic β cell. In some cases, cell sorting as described herein can comprise magnetic-activated flow cytometry, where magnetic antibody or other ligand is used to label cells of different types, and the differences in magnetic properties can be used for cell sorting.

The cells dissociated from the first cell cluster can be cultured in a medium for re-aggregating to a second cell cluster. The medium can comprise Connought Medical Research Laboratories 1066 supplemented islet media (CMRLS). In some cases, the suitable culture medium comprises a component of CMRLS (e.g., supplemental zinc). The CMRLS can be supplemented, e.g., with serum (e.g., human serum, human platelet lysate, fetal bovine serum, or serum replacements such as Knockout Serum Replacement).

The medium can comprise one or more compounds that regulate certain signaling pathways in cells. For example, the medium can comprise a thyroid hormone signaling pathway activator, a transforming growth factor β (TGF-β) signaling pathway inhibitor, or both.

The thyroid hormone signaling pathway activator in the medium used herein can be triiodothyronine (T3). In some cases, the thyroid hormone signaling pathway activator can be an analog or derivative of T3. Non-limiting exemplary analogs of T3 include selective and non-selective thyromimetics, TRβ selective agonist-GC-1, GC-24,4-Hydroxy-PCB 106, MB07811, MB07344,3,5-diiodothyropropionic acid (DITPA); the selective TR-β agonist GC-1; 3-Iodothyronamine (T(1)AM) and 3,3′,5-triiodothyroacetic acid (Triac) (bioactive metabolites of the hormone thyroxine (T(4)); KB-2115 and KB-141; thyronamines; SKF L-94901; DIBIT; 3′-AC-T2; tetraiodothyroacetic acid (Tetrac) and triiodothyroacetic acid (Triac) (via oxidative deamination and decarboxylation of thyroxine (T4) and triiodothyronine (T3) alanine chain), 3,3′,5′-triiodothyronine (rT3) (via T4 and T3 deiodination), 3,3′-diiodothyronine (3,3′-T2) and 3,5-diiodothyronine (T2) (via T4, T3, and rT3 deiodination), and 3-iodothyronamine (T1AM) and thyronamine (TOAM) (via T4 and T3 deiodination and amino acid decarboxylation), as well as for TH structural analogs, such as 3,5,3′-triiodothyropropionic acid (Triprop), 3,5-dibromo-3-pyridazinone-1-thyronine (L-940901), N-[3,5-dimethyl-4-(4′-hydroxy-3′-isopropylphenoxy)-phenyl]-oxamic acid (CGS 23425), 3,5-dimethyl-4-[(4′-hydroxy-3′-isopropylbenzyl)-phenoxy]acetic acid (GC-1), 3,5-dichloro-4-[(4-hydroxy-3-isopropylphenoxy)phenyl]acetic acid (KB-141), and 3,5-diiodothyropropionic acid (DITPA). In some cases, the thyroid hormone signaling pathway activator is a prodrug or prohormone of T3, such as T4 thyroid hormone (e.g., thyroxine or L-3,5,3′,5′-tetraiodothyronine). The thyroid hormone signaling pathway activator can also be an iodothyronine composition described in U.S. Pat. No. 7,163,918, which is incorporated by reference herein in its entirety.

The concentration of the thyroid hormone signaling pathway activator in the medium can be in a range suitable for cell aggregation. In some cases, the concentration of the thyroid hormone signaling pathway activator in the medium is from about 0.1 μM to about 10 μM, such as from about 0.5 μM to about 2 μM, from about 0.8 μM to about 1.5 μM, from about 0.9 μM to about 1.5 μM, from about 0.9 μM to about 1.2 μM, or from about 0.9 μM to about 1.2 μM. In some cases, the contraction of the thyroid hormone signaling pathway activator in the medium is at least about 0.1 μM, 0.2 μM, 0.4 μM, 0.8 μM, 0.9 μM, 1 μM, 1.1 μM, 1.2 μM, 1.3 μM, 1.4 μM, 1.5 μM, 2 μM, 3 μM, 4 μM, 5 μM, 6 μM, 7 μM, 8 μM, 9 μM, or 10 μM. In some case, the contraction of the thyroid hormone signaling pathway activator (e.g., T3) in the medium is about 1 μM.

The TGF-β signaling pathway inhibitor used in the medium herein can be an inhibitor of TGF-β receptor type I kinase (TGF-β RI) signaling. The TGF-β signaling pathway inhibitor can be an activin receptor-like kinase-5 (Alk5) inhibitor, e.g., ALK5 inhibitor II (CAS 446859-33-2, an ATP-competitive inhibitor of TGF-β RI kinase, also known as RepSox, IUPAC Name: 2-[5-(6-methylpyridin-2-yl)-1H-pyrazol-4-yl]-1,5-naphthyridine). In some cases, the TGF-β signaling pathway inhibitor is an analog or derivative of ALK5 inhibitor II, including those described in in U.S. Patent Publication Nos. 2012/0021519, 2010/0267731, 2009/0186076, and 2007/0142376, which are incorporated by reference herein in their entireties. In some cases, examples of TGF-β signaling pathway inhibitor that can be used in the medium herein also include D 4476, SB431542, A-83-01, also known as 3-(6-Methyl-2-pyridinyl)-N-phenyl-4-(4-quinolinyl)-1H-pyrazole-1-carbothioamide; 2-(3-(6-Methylpyridin-2-yl)-1H-pyrazol-4-yl)-1, 5-naphthyridine, Wnt3a/BIO, BMP4, GW788388 (-(4-[3-(pyridin-2-yl)-1H-pyrazol-4-yl]pyridm-2-yl}-N-(tetrahydro-2H-pyran-4-yl)benzamide), SMI 6, 1N-1 130 (3-((5-(6-methylpyridin-2-yl)-4-(quinoxalin-6-yl)-1H-imidazol-2-yl)methyl)benzamide, GW6604 (2-phenyl-4-(3-pyridin-2-yl-1H-pyrazol-4-yl)pyridine), SB-505124 (2-(5-benzo[1,3]dioxol-5-yl-2-tert-butyl-3H-imidazol-4-yl)-6-methylpyridine hydrochloride), SU5416, lerdelimumb (CAT-152), metelimumab (CAT-192), GC-1008, ID11, AP-12009, AP-1 1014, LY550410, LY580276, LY364947, LY2109761, SD-208, SM16, NPC-30345, KÏ26894, SB-203580, SD-093, ALX-270-448, EW-7195, SB-525334, ΓN-1233, SKI2162, Gleevec, 3,5,7,2′,4′-pentahydroxyfiavone (Morin), activin-M108A, P144, soluble TBR2-Fc, pyrimidine derivatives and indolinones. Inhibition of the TGF-β/activin pathway can have similar effects. Thus, any inhibitor (e.g., upstream or downstream) of the TGF-β/activin pathway can be used in combination with, or instead of, TGF-β/ALK5 inhibitors as described herein. Exemplary TGF-β/activin pathway inhibitors include, but are not limited to, TGF-β receptor inhibitors, inhibitors of SMAD 2/3 phosphorylation, inhibitors of the interaction of SMAD 2/3 and SMAD 4, and activators/agonists of SMAD 6 and SMAD 7. Furthermore, the categorizations described herein are merely for organizational purposes and one of skill in the art would know that compounds can affect one or more points within a pathway, and thus compounds may function in more than one of the defined categories. TGF-β receptor inhibitors may include any inhibitors of TGF signaling in general or inhibitors specific for TGF-β receptor (e.g., ALK5) inhibitors, which can include antibodies to, dominant negative variants of, and siRNA and antisense nucleic acids that suppress expression of, TGF-β receptors.

The concentration of the TGF-β signaling pathway inhibitor in the medium can be in a range suitable for cell aggregation. In some cases, the concentration of the TGF-β signaling pathway inhibitor in the medium is from about 1 μM to about 50 μM, such as from about 5 μM to about 15 μM, from about 8 μM to about 12 μM, or from about 9 μM to about 11 μM. In some cases, the contraction of the TGF-β signaling pathway inhibitor in the medium is at least about 1 μM, 5 μM, 8 μM, 9 μM, 10 μM, 11 μM, 12 μM, 13 μM, 14 μM, 15 μM, 20 μM, 25 μM, 30 μM, 35 μM, 40 μM, 45 μM, or 50 μM. In some case, the contraction of the TGF-β signaling pathway inhibitor (e.g., Alk5 inhibitor II) in the medium is about 10 μM.

The medium used to culture the cells dissociated from the first cell cluster can be xeno-free. A xeno-free medium for culturing cells and/or cell clusters of originated from an animal can have no product from other animals. In some cases, a xeno-free medium for culturing human cells and/or cell clusters can have no products from any non-human animals. For example, a xeno-free medium for culturing human cells and/or cell clusters can comprise human platelet lysate (PLT) instead of fetal bovine serum (FBS). For example, a medium can comprise from about 1% to about 20%, from about 5% to about 15%, from about 8% to about 12%, from about 9 to about 11% serum. In some cases, medium can comprise about 10% of serum. In some cases, the medium can be free of small molecules and/or FBS. For example, a medium can comprise MCDB131 basal medium supplemented with 2% BSA. In some cases, the medium is serum-free. In some examples, a medium can comprise no exogenous small molecules or signaling pathway agonists or antagonists, such as, growth factor from fibroblast growth factor family (FGF, such as FGF2, FGF8B, FGF 10, or FGF21), Sonic Hedgehog Antagonist (such as Sant1, Sant2, Sant 4, Sant4, Cur61414, forskolin, tomatidine, AY9944, triparanol, cyclopamine, or derivatives thereof), Retinoic Acid Signaling agonist (e.g., retinoic acid, CD1530, AM580, TTHPB, CD437, Ch55, BMS961, AC261066, AC55649, AM80, BMS753, tazarotene, adapalene, or CD2314), inhibitor of Rho-associated, coiled-coil containing protein kinase (ROCK) (e.g., Thiazovivin, Y-27632, Fasudil/HA1077, or 14-1152), activator of protein kinase C (PKC) (e.g., phorbol 12,13-dibutyrate (PDBU), TPB, phorbol 12-myristate I3-acetate, bryostatin 1, or derivatives thereof), antagonist of TGF beta super family (e.g, Alk5 inhibitor II (CAS 446859-33-2), A83-01, SB431542, D4476, GW788388, LY364947, LY580276, SB505124, GW6604, SB-525334, SD-208, SB-505124, or derivatives thereof), inhibitor of Bone Morphogenic Protein (BMP) type 1 receptor (e.g., LDN193189 or derivatives thereof), thyroid hormone signaling pathway activator (e.g., T3 or derivatives thereof), gamma-secretase inhibitor (e.g., XXI, DAPT, or derivatives thereof), activator of TGF-β signaling pathway (e.g., WNT3a or Activin A) growth factor from epidermal growth factor (EGF) family (e.g., betacellulin or EGF), broad kinase (e.g., staurosporine or derivatives thereof), non-essential amino acids, vitamins or antioxidants (e.g., cyclopamine, vitamin D, vitamin C, vitamin A, or derivatives thereof), or other additions like N-acetyl cysteine, zinc sulfate, or heparin. In some cases, the reaggregation medium can comprise no exogenous extracellular matrix molecule. In some cases, the reaggregation medium does not comprise Matrigel™ In some cases, the reaggregation medium does not comprise other extracellular matrix molecules or materials, such as, collagen, gelatin, poly-L-lysine, poly-D-lysine, vitronectin, laminin, fibronectin, PLO laminin, fibrin, thrombin, and RetroNectin and mixtures thereof, for example, or lysed cell membrane preparations.

A person of ordinary skill in the art will appreciate that that the concentration of BSA supplemented into the medium may vary. For example, a medium (e.g., MCDB131) can comprise about 0.01%, 0.05%, 0.1%, 1%, about 2%, about 3%, about 4%, about 5%, about 10%, or about 15% BSA. The medium used (e.g., MCDB131 medium) can contain components not found in traditional basal media, such as trace elements, putrescine, adenine, thymidine, and higher levels of some amino acids and vitamins. These additions can allow the medium to be supplemented with very low levels of serum or defined components. The medium can be free of proteins and/or growth factors, and may be supplemented with EGF, hydrocortisone, and/or glutamine.

The medium can comprise one or more extracellular matrix molecules (e.g., extracellular proteins). Non-limiting exemplary extracellular matrix molecules used in the medium can include collagen, placental matrix, fibronectin, laminin, merosin, tenascin, heparin, heparin sulfate, chondroitin sulfate, dermatan sulfate, aggrecan, biglycan, thrombospondin, vitronectin, and decorin. In some cases, the medium comprises laminin, such as LN-332. In some cases, the medium comprises heparin.

The medium can be changed periodically in the culture, e.g., to provide optimal environment for the cells in the medium. When culturing the cells dissociated from the first cell cluster for re-aggregation, the medium can be changed at least or about every 4 hours, 12 hours, 24 hours, 48 hours, 3 days or 4 days. For example, the medium can be changed about every 48 hours.

Cells dissociated from the first cell cluster can be seeded in a container for re-aggregation. The seeding density can correlate with the size of the re-aggregated second cell cluster. The seeding density can be controlled so that the size of the second cell cluster can be similar to an endogenous pancreatic islet. In some cases, the seeding density is controlled so that the size of the second cell cluster can be from about 75 μm to about 250 μm. Cells dissociated from the first cell cluster can be seeded at a density of from about 0.1 million cells per mL to about 10 million cells per mL, e.g., from about 0.5 million cells per mL to about 1.5 million cells per mL, from about 0.8 million cells per mL to about 1.2 million cells per mL, from about 0.9 million cells per mL to about 1.1 million cells per mL, from about 2 million cells per mL to about 3 million cells per mL. In some cases, the cells dissociated from the first cell cluster can be seeded at a density of about 1 million cells per mL. In some cases, the cells dissociated from the first cell cluster can be seeded at a density of about 1.5 million cells per mL. In some cases, the cells dissociated from the first cell cluster can be seeded at a density of about 2 million cells per mL. In some cases, the cells dissociated from the first cell cluster can be seeded at a density of about 2.5 million cells per mL. In some cases, the cells dissociated from the first cell cluster can be seeded at a density of about 3 million cells per mL.

The cell dissociated from the first cell cluster can be cultured in a culture vessel. The culture vessel can be suitable for culturing a suspension of culture of cells. The culture vessel used for culturing the cells or cell clusters herein can include, but is not limited to: flask, flask for tissue culture, dish, petri dish, dish for tissue culture, multi dish, micro plate, micro-well plate, multi plate, multi-well plate, micro slide, chamber slide, tube, tray, culture bag, and roller bottle, stir tank bioreactors, or polymer (e.g., biopolymer or gel) encapsulation as long as it is capable of culturing the cells therein. The cells and/or cell clusters can be cultured in a volume of at least or about 0.2 ml, 0.5 ml, 1 ml, 5 ml, 10 ml, 20 ml, 30 ml, 40 ml, 50 ml, 100 ml, 150 ml, 200 ml, 250 ml, 300 ml, 350 ml, 400 ml, 450 ml, 500 ml, 600 ml, 800 ml, 1000 ml, 1500 ml, 2000 ml, 3000 ml or any range derivable therein, depending on the needs of the culture.

In some cases, cells can be cultured under dynamic conditions (e.g., under conditions in which the cells are subject to constant movement or stirring while in the suspension culture). For dynamic culturing of cells, the cells can be cultured in a container (e.g., an non-adhesive container such as a spinner flask (e.g., of 200 ml to 3000 ml, for example 250 ml; of 100 ml; or in 125 ml Erlenmeyer), which can be connected to a control unit and thus present a controlled culturing system. In some cases, cells can be cultured under non-dynamic conditions (e.g., a static culture) while preserving their proliferative capacity. For non-dynamic culturing of cells, the cells can be cultured in an adherent culture vessel. An adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, vitronectin, laminin, fibronectin, PLO laminin, fibrin, thrombin, and RetroNectin and mixtures thereof, for example, Matrigel™, and lysed cell membrane preparations.

Medium in a dynamic cell culture vessel (e.g., a spinner flask) can be stirred (e.g., by a stirrer). The spinning speed can correlate with the size of the re-aggregated second cell cluster. The spinning speed can be controlled so that the size of the second cell cluster can be similar to an endogenous pancreatic islet. In some cases, the spinning speed is controlled so that the size of the second cell cluster can be from about 75 μm to about 250 μm. The spinning speed of a dynamic cell culture vessel (e.g., a spinner flask) can be about 20 rounds per minute (rpm) to about 100 rpm, e.g., from about 30 rpm to about 90 rpm, from about 40 rpm to about 60 rpm, from about 45 rpm to about 50 rpm. In some cases, the spinning speed can be about 50 rpm.

The cells dissociated from the first cell cluster can be cultured for a period of time to allow them for re-aggregating. The cells dissociated from the first cell cluster can be cultured for at least 12 hours, 24 hours, 1 day, 2 days, 3 days, 4 days, 5 days, 6 days, 7 days 8 days, 9 days 10 days, 15 days, 20 days, 25 days, or 30 days. In some cases, the cells dissociated from the first cell cluster can be cultured for at least 4 days.

The methods herein can also be used to enrich cells resembling endogenous cells, e.g., endogenous mature pancreatic β cells in a cell cluster. The methods can comprise dissociating a first cell cluster and re-aggregating the cells from the first cluster to a second cluster. The second cluster can comprise more cells resembling endogenous mature pancreatic β cells compared to the first cluster. The dissociating and re-aggregating can be performed using any methods and reagents disclosed through the application.

After re-aggregation, the second cell cluster can comprise more cells expressing one or more markers of an endogenous cell compared to the first cell cluster. For example, the second cluster can comprise more cells expressing one or more markers of an endogenous mature pancreatic β cell, the markers including insulin, C-peptide, PDX1, NKX6.1, CHGA, MAFA, ZNT8, PAX6, NEUROD1, glucokinase (GCK), SLC2A, PCSK1, KCNJ11, ABCC8, SLC30A8, SNAP25, RAB3A, GAD2, and PTPRN, compared to the first cell cluster. In some cases, the second cluster can comprise more cells expressing CHGA. In some cases, the second cluster can comprise more cells expressing NKX6.1. In some cases, the second cluster can comprise more cells expressing C-peptide. In some cases, the second cluster can comprise more cells expressing NKX6.1 and C-peptide. In some cases, the second cluster can comprise more cells expressing CHGA, NKX6.1 and C-peptide.

After re-aggregation, the second cell cluster can have a smaller size (e.g., a smaller diameter) compared to the first cell cluster. The smaller size can allow better exchange of molecules between the cell cluster and the surrounding environment. For example, a smaller size can allow better diffusion of molecules (e.g., reagents, gas, and/or nutrition) from the medium to the cells in a cell cluster. Thus, being in a smaller size, the second cell cluster can exchange molecules with the surrounding environment in a more efficient way compared to the first cell cluster. Thus the second cell cluster can have less dead cells (e.g., cells died due to insufficient nutrition and/or gas) compared to the first cell cluster.

A method provided herein can enrich endocrine cells, e.g., cells expressing chromogranin A (CHGA). For examples, a percentage of cells in the second cell cluster that express chromogranin A is at least 1.2, at least 1.3, at least 1.4, or at least 1.5 times more than a percentage of cells in the first cell cluster that express chromogranin A. In some cases, the second cell cluster comprises at least about 70%, at least about 80%, at least about 90%, at least about 95%, at least about 99%, or 100% cells expressing CHGA. In some cases, at least about 85% cells in the second cell cluster can express CHGA. In some cases, the second cell cluster can comprise about 90% cell expressing CHGA. In some cases, the second cell cluster can comprise about 95% cells expressing CHGA. In certain cases, all cells in the second cell cluster can express CHGA.

A method provided herein can generate or enrich pancreatic β cell. For example, the second cell cluster comprises at least one pancreatic β cell, e.g., at least one non-native pancreatic β cell. For examples, a percentage of cells in the second cell cluster that express both NKX6.1 and C-peptide is at least 1.5, at least 1.75, or at least 2 times more than a percentage of cells in the first cell cluster that express both NKX6.1 and C-peptide. In some cases, the second cell cluster comprises at least about 35%, at least about 40%, at least about 50%, at least about 60%, at least about 70%, at least about 80%, at least about 90%, at least about 95%, at least about 99%, or 100% cells expressing NKX6.1 and C-peptide. In some cases, at least about 35% cells in the second cell cluster can express NKX6.1 and C-peptide. In some cases, a cell cluster can comprise about 60% cells expressing NKX6.1 and C-peptide. In some cases, the second cell cluster can comprise about 75% cell expressing NKX6.1 and C-peptide. In some cases, all cells in the second cell cluster can express NKX6.1 and C-peptide. In some cases, at least about 70% of the at least one non-native pancreatic β cell in the second cell cluster express chromogranin A as measured by flow cytometry. In some cases, at least about 25% of the at least one non-native pancreatic β cell in the second cell cluster express NKX6.1 and C-peptide as measured by flow cytometry.

A method provided herein can reduce or eliminate stem cells or precursor cells of a pancreatic endocrine cell. In some cases, a percentage of cells in the second cell cluster that express SOX2 is at least 2, at least 3, at least 5, or at least 10 times lower than a percentage of cells in the first cell cluster that express LIN28, Ki67, SOX2, or SOX9. For example, the second cell cluster can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing LIN28. In some examples, the second cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing Ki67. For example, the second cell cluster can comprise at most 3% cells, at most about 2% cells, at most about 1% cells, at most about 0.5% cells, at most about 0.1% cells, at most about 0.05% cells, at most about 0.01% cells, or no cells expressing SOX2. In some cases, the second cell cluster can comprise about 1% cells expressing SOX2. In some cases, the second cell cluster can comprise about 0.6% cells expressing SOX2. In some cases, the second cell cluster can comprise about 0.3% cells expressing SOX2. In some cases, the second cell cluster can comprise about 0.1% cells expressing SOX2. For examples, the second cell cluster can comprise at most 10% cells, at most about 8% cells, at most about 6% cells, at most about 5% cells, at most about 2% cells, at most about 1% cells, at most about 0.5% cells, at most about 0.1% cells, at most about 0.05% cells, at most about 0.01% cells, or no cells expressing SOX9. In some cases, the second cell cluster can comprise about 2% cells expressing SOX9. In some cases, the second cell cluster can comprise about 6% cells expressing SOX9. In some cases, the second cell cluster can comprise about 1.2% cells expressing SOX9.

The second cell cluster can also function more similarly to an endogenous pancreatic islet compared to the first cell cluster. The second cell cluster can have a higher insulin content than the first cell cluster, for instance, at least 1.1, at least 1.25 or at least 1.5 times higher insulin content as compared to the first cell cluster. The second cluster can exhibit a greater in vitro GSIS than the first cell cluster, as measured by stimulation indexes. The second cluster can also exhibit a greater in vivo GSIS than the first cell cluster, as measured by stimulation indexes. In some cases, the second cluster can exhibit a greater in vitro GSIS and a greater in vivo GSIS compared to the first cell cluster, as measured by stimulation indexes. For example, the second cell cluster can secrete more insulin than the first cell cluster under the same stimulation conditions. The second cell cluster can also exhibit insulin secretion response to a potassium challenge (K⁺), e.g., a concentration of KCl, e.g., 30 mM KCl.

In some cases, the method provided herein can retain a large percentage of cells from the first cell cluster in the second cell cluster, e.g., pancreatic β cells or endocrine cells. For example, at least about 95%, at least about 98%, or at least about 99% of cells that express both NKX6.1 and C-peptide in the first cell cluster can be retained in the second in vitro cell cluster. In some cases, at most about 5%, at most about 2%, at most about 1%, at most about 0.5%, or at most about 0.1% of cells that express both NKX6.1 and C-peptide in the first cell cluster are lost during the dissociation and reaggregation process.

In some cases, the cell cluster as described herein is generated from any starting cell population in vitro. For example, the starting cell can include, without limitation, insulin-positive endocrine cells (e.g., chromogranina A-positive cells) or any precursor thereof, such as a Nkx6.1-positive pancreatic progenitor cell, a Pdx1-positive pancreatic progenitor cell, and a pluripotent stem cell, an embryonic stem cell, and induced pluripotent stern cell. In some cases, the method include differentiation of a reprogrammed cell, a partially reprogrammed cell (e.g., a somatic cell, e.g., a fibroblast which has been partially reprogrammed such that it exists in an intermediate state between an induced pluripotency cell and the somatic cell from which it has been derived), a transdifferentiated cell. In some cases, the cell cluster comprising the pancreatic β cell disclosed herein can be differentiated in vitro from an insulin-positive endocrine cell or a precursor thereof. In some cases, the cell cluster comprising the pancreatic β cell is differentiated in vitro from a precursor selected from the group consisting of a NKX6.1-positive pancreatic progenitor cell, a Pdx1-positive pancreatic progenitor cell, and a pluripotent stem cell. In some cases, the pluripotent stem cell is selected from the group consisting of an embryonic stem cell and induced pluripotent stem cell. As discussed above, the non-native pancreatic β cells can also be referred to as stem cell-derived β cells (SC-β cells) as they can be derived from stem cells in vitro. In some cases, the SC-β cell or the pluripotent stem cell from which the SC-β cell is derived is human. In some cases, the SC-β cell is human.

One aspect of the present disclosure provides a method of generating non-native pancreatic β cells. In some cases, the method can be any currently available protocol, such as those described in U.S. patent application Ser. Nos. 14/684,129 and 14/684,101, each of which is incorporated herein by its entirety. Aspects of the disclosure involve definitive endoderm cells, Definitive endoderm cells of use herein can be derived from any source or generated in accordance with any suitable protocol. In some aspects, pluripotent stem cells, e.g., iPSCs or hESCs, are differentiated to endoderm cells. In some aspects, the endoderm cells (stage 1) are further differentiated, e.g., to primitive gut tube cells (stage 2), Pdx1-positive pancreatic progenitor cells (stage 3), NKX6.1-positive pancreatic progenitor cells (stage 4), or Ngn3-positive endocrine progenitor cells or insulin-positive endocrine cells (stage 5), followed by induction or maturation to SC-β cells (stage 6).

In some cases, definitive endoderm cells can be obtained by differentiating at least some pluripotent cells in a population into definitive endoderm cells, e.g., by contacting a population of pluripotent cells with i) at least one growth factor from the TGF-β superfamily, and ii) a WNT signaling pathway activator, to induce the differentiation of at least some of the pluripotent cells into definitive endoderm cells, wherein the definitive endoderm cells express at least one marker characteristic of definitive endoderm.

Any growth factor from the TGF-β superfamily capable of inducing the pluripotent stem cells to differentiate into definitive endoderm cells (e.g., alone, or in combination with a WNT signaling pathway activator) can be used in the method provided herein. In some cases, the at least one growth factor from the TGF-β superfamily comprises Activin A. In some cases, the at least one growth factor from the TGF-β superfamily comprises growth differentiating factor 8 (GDF8). Any WNT signaling pathway activator capable of inducing the pluripotent stem cells to differentiate into definitive endoderm cells (e.g., alone, or in combination with a growth factor from the TGF-β superfamily) can be used in the method provided herein. In some cases, the WNT signaling pathway activator comprises CHIR99Q21. In some cases, the WNT signaling pathway activator comprises Wnt3a recombinant protein.

In some cases, differentiating at least some pluripotent cells in a population into definitive endoderm cells is achieved by a process of contacting a population of pluripotent cells with i) Activin A, and ii) CHIR99021 for a period of 3 days, to induce the differentiation of at least some of the pluripotent cells in the population into definitive endoderm cells, wherein the definitive endoderm cells express at least one marker characteristic of definitive endoderm.

In some cases, a definitive endoderm cell produced by the methods as disclosed herein expresses at least one marker selected from the group consisting of: Nodal, Tmprss2, Tmem30b, St14, Spink3, Sh3g12, Ripk4, Rab1S, Npnt, Clic6, CldnS, Cacnalb, Bnipl, Anxa4, Emb, FoxA1, Sox17, and Rbm35a, wherein the expression of at least one marker is upregulated to by a statistically significant amount in the definitive endoderm cell relative to the pluripotent stem cell from which it was derived. In some cases, a definitive endoderm cell produced by the methods as disclosed herein does not express by a statistically significant amount at least one marker selected the group consisting of: Gata4, SPARC, AFP and Dab2 relative to the pluripotent stem cell from which it was derived. In some cases, a definitive endoderm cell produced by the methods as disclosed herein does not express by a statistically significant amount at least one marker selected the group consisting of: Zicl, Pax6, Flkl and CD31 relative to the pluripotent stem cell from which it was derived. In some cases, a definitive endoderm cell produced by the methods as disclosed herein has a higher level of phosphorylation of Smad2 by a statistically significant amount relative to the pluripotent stem cell from which it was derived. In some cases, a definitive endoderm cell produced by the methods as disclosed herein has the capacity to form gut tube in vivo. In some cases, a definitive endoderm cell produced by the methods as disclosed herein can differentiate into a cell with morphology characteristic of a gut cell, and wherein a cell with morphology characteristic of a gut cell expresses FoxA2 and/or Claudin6, In some cases, a definitive endoderm cell produced by the methods as disclosed herein can be further differentiated into a cell of endoderm origin.

In some cases, a population of pluripotent stem cells are cultured in the presence of at least one 3 cell maturation factor prior to any differentiation or during the first stage of differentiation. One can use any pluripotent stem cell, such as a human pluripotent stem cell, or a human iPS cell or any of pluripotent stem cell as discussed herein or other suitable pluripotent stem cells. In some cases, a β cell maturation factor as described herein can be present in the culture medium of a population of pluripotent stem cells or may be added in bolus or periodically during growth (e.g. replication or propagation) of the population of pluripotent stem cells. In certain examples, a population of pluripotent stem cells can be exposed to at least one β cell maturation factor prior to any differentiation. In other examples, a population of pluripotent stem cells may be exposed to at least one β cell maturation factor during the first stage of differentiation.

Aspects of the disclosure involve primitive gut tube cells. Primitive gut tube cells of use herein can be derived from any source or generated in accordance with any suitable protocol. In some aspects, definitive endoderm cells are differentiated to primitive gut tube cells. In some aspects, the primitive gut tube cells are further differentiated, e.g., to Pdx1-positive pancreatic progenitor cells, NKX6.1-positive pancreatic progenitor cells, Ngn3-positive endocrine progenitor cells, insulin-positive endocrine cells, followed by induction or maturation to SC-β cells.

In some cases, primitive gut tube cells can be obtained by differentiating at least some definitive endoderm cells in a population into primitive gut tube cells, e.g., by contacting definitive endoderm cells with at least one growth factor from the fibroblast growth factor (FGF) family, to induce the differentiation of at least some of the definitive endoderm cells into primitive gut tube cells, wherein the primitive gut tube cells express at least one marker characteristic of primitive gut tube cells.

Any growth factor from the FGF family capable of inducing definitive endoderm cells to differentiate into primitive gut tube cells (e.g., alone, or in combination with other factors) can be used in the method provided herein. In some cases, the at least one growth factor from the FGF family comprises keratinocyte growth factor (KGF). In some cases, the at least one growth factor from the FGF family comprises FGF2. In some cases, the at least one growth factor from the FGF family comprises FGF8B. In some cases, the at least one growth factor from the FGF family comprises FGF 10. In some cases, the at least one growth factor from the FGF family comprises FGF21.

In some cases, primitive gut tube cells can be obtained by differentiating at least some definitive endoderm cells in a population into primitive gut tube cells, e.g., by contacting definitive endoderm cells with KGF for a period of 2 days, to induce the differentiation of at least some of the definitive endoderm cells into primitive gut tube cells.

Aspects of the disclosure involve Pdx1-positive pancreatic progenitor cells. Pdx1-positive pancreatic progenitor cells of use herein can be derived from any source or generated in accordance with any suitable protocol. In some aspects, primitive gut tube cells are differentiated to Pdx1-positive pancreatic progenitor cells. In some aspects, the Pdx1-positive pancreatic progenitor cells are further differentiated, e.g., NKX6.1-positive pancreatic progenitor cells, Ngn3-positive endocrine progenitor cells, insulin-positive endocrine cells, followed by induction or maturation to SC-β cells,

In some aspects, Pdx1-positive pancreatic progenitor cells can be obtained by differentiating at least some primitive gut tube cells in a population into Pdx1-positive pancreatic progenitor cells, e.g., by contacting primitive gut tube cells with i) at least one bone morphogenic protein (BMP) signaling pathway inhibitor, ii) at least one growth factor from the FGF family, in) at least one SHH pathway inhibitor, iv) at least one retinoic acid (RA) signaling pathway activator; and v) at least one protein kinase C activator, to induce the differentiation of at least some of the primitive gut tube cells into Pdx1-positive pancreatic progenitor cells, wherein the Pdx1-positive pancreatic progenitor cells express Pdx1. In some cases, Pdx1-positive pancreatic progenitor cells can be obtained by differentiating at least some primitive gut tube cells in a population into Pdx1-positive pancreatic progenitor cells, e.g., by contacting primitive gut tube cells with i) at least one growth factor from the FGF family, and ii) at least one retinoic acid (RA) signaling pathway activator, to induce the differentiation of at least some of the primitive gut tube cells into Pdx1-positive pancreatic progenitor cells, wherein the Pdx1-positive pancreatic progenitor cells express Pdx1.

Any BMP signaling pathway inhibitor capable of inducing primitive gut tube cells to differentiate into Pdx1-positive pancreatic progenitor cells (e.g., alone, or with any combination of at least one growth factor from the FGF family, at least one SHH pathway inhibitor, at least one retinoic acid signaling pathway activator, and at least one protein kinase C activator) can be used in the method provided herein. In some cases, the BMP signaling pathway inhibitor comprises LDN193189.

Any growth factor from the FGF family capable of inducing primitive gut tube cells to differentiate into Pdx1-positive pancreatic progenitor cells (e.g., alone, or with any combination of at least one BMP signaling pathway inhibitor, at least one SHH pathway inhibitor, at least one retinoic acid signaling pathway activator, and at least one protein kinase C activator) can be used. In some cases, the at least one growth factor from the FGF family comprises keratinocyte growth factor (KGF). In some cases, the at least one growth factor from the FGF family is selected from the group consisting of FGF2, FGF8B, FGF 1 0, and FGF21.

Any SHH pathway inhibitor capable of inducing primitive gut tube cells to differentiate into Pdx1-positive pancreatic progenitor cells (e.g., alone, or with any combination of at least one BMP signaling pathway inhibitor, at least one growth factor from the FGF family, at least one retinoic acid signaling pathway activator, and at least one protein kinase C activator) can be used. In some cases, the SHH pathway inhibitor comprises Sant 1.

Any RA signaling pathway activator capable of inducing primitive gut tube cells to differentiate into Pdx1-positive pancreatic progenitor cells (e.g., alone, or with any combination of at least one BMP signaling pathway inhibitor, at least one growth factor from the FGF family, at least one SHH pathway inhibitor, and at least one protein kinase C activator) can be used. In some cases, the RA signaling pathway activator comprises retinoic acid.

Any PKC activator capable of inducing primitive gut tube cells to differentiate into Pdx1-positive pancreatic progenitor cells (e.g., alone, or with any combination of at least one BMP signaling pathway inhibitor, at least one growth factor from the FGF family, at least one SHH pathway inhibitor, and at least one RA signaling pathway activator) can be used. In some cases, the PKC activator comprises PdbU. In some cases, the PKC activator comprises TPB.

In some cases, Pdx1-positive pancreatic progenitor cells can be obtained by differentiating at least some primitive gut tube cells in a population into Pdx1-positive pancreatic progenitor cells, e.g., by contacting primitive gut tube cells with retinoic acid, KGF, Sant1, LDN193189, PdBU for a period of 2 days. In some cases, Pdx1-positive pancreatic progenitor cells can be obtained by differentiating at least some primitive gut tube cells in a population into Pdx1-positive pancreatic progenitor cells, e.g., by contacting primitive gut tube cells with retinoic acid and KGF for a period of 2 days. In some cases, Pdx1-positive pancreatic progenitor cells can be obtained by differentiating at least some primitive gut tube cells in S3 medium

Aspects of the disclosure involve NKX6.1-positive pancreatic progenitor cells. NKX6.1-positive pancreatic progenitor cells of use herein can be derived from any source or generated in accordance with any suitable protocol. In some aspects, Pdx1-positive pancreatic progenitor cells are differentiated to NKX6.1-positive pancreatic progenitor cells. In some aspects, the NKX6.1-positive pancreatic progenitor cells are further differentiated, e.g., to Ngn3-positive endocrine progenitor cells, or insulin-positive endocrine cells, followed by induction or maturation to SC-β cells.

In some aspects, a method of producing a NKX6.1-positive pancreatic progenitor cell from a Pdx 1-positive pancreatic progenitor cell comprises contacting a population of cells (e.g., under conditions that promote cell clustering) comprising Pdx1-positive pancreatic progenitor cells with at least two β cell-maturation factors comprising a) at least one growth factor from the fibroblast growth factor (FGF) family, b) a sonic hedgehog pathway inhibitor, and optionally c) a low concentration of a retinoic acid (RA) signaling pathway activator, to induce the differentiation of at least one Pdx1-positive pancreatic progenitor cell in the population into NKX6.1-positive pancreatic progenitor cells, wherein the NKX6.1-positive pancreatic progenitor cells expresses NKX6.1.

In some cases, the Pdx1-positive, NKX6.1-positive pancreatic progenitor cells are obtained by contacting Pdx1-positive pancreatic progenitor cells under conditions that promote cell clustering with i) at least one growth factor from the FGF family, ii) at least one SHH pathway inhibitor, and optionally iii) low concentrations of a RA signaling pathway activator, to induce the differentiation of at least some of the Pdx1-positive pancreatic progenitor cells into Pdx1-positive, NKX6.1-positive pancreatic progenitor cells, wherein the Pdx1-positive, NKX6.1-positive pancreatic progenitor cells expresses Pdx1 and NKX6.1. In some cases, the Pdx1-positive, NKX6.1-positive pancreatic progenitor cells are obtained by contacting Pdx1-positive pancreatic progenitor cells under conditions that promote cell clustering with i) at least one growth factor from the FGF family, ii) at least one SHH pathway inhibitor, and optionally iii) low concentrations of a RA signaling pathway activator, iv) ROCK inhibitor, and v) at least one growth factor from the TGF-β superfamily, to induce the differentiation of at least some of the Pdx1-positive pancreatic progenitor cells into Pdx1-positive, NKX6.1-positive pancreatic progenitor cells. In some cases, the Pdx1-positive, NKX6.1-positive pancreatic progenitor cells are obtained by contacting Pdx1-positive pancreatic progenitor cells under conditions that promote cell clustering with at least one growth factor from the FGF family.

In some cases, the Pdx1-positive pancreatic progenitor cells are produced from a population of pluripotent cells. In some cases, the Pdx1-positive pancreatic progenitor cells are produced from a population of iPS cells. In some cases, the Pdx1-positive pancreatic progenitor cells are produced from a population of ESC cells. In some cases, the Pdx1-positive pancreatic progenitor cells are produced from a population of definitive endoderm cells. In some cases, the Pdx1-positive pancreatic progenitor cells are produced from a population of primitive gut tube cells.

Any growth factor from the FGF family capable of inducing Pdx1-positive pancreatic-progenitor cells to differentiate into NKX6.1-positive pancreatic progenitor cells (e.g., alone, or with any combination of at least one SHH pathway inhibitor, or optionally at least one retinoic acid signaling pathway activator) can be used in the method provided herein. In some cases, the at least one growth factor from the FGF family comprises keratinocyte growth factor (KGF). In some cases, the at least one growth factor from the FGF family is selected from the group consisting of FGF2, FGF8B, FGF 10, and FGF21.

Any SHH pathway inhibitor capable of inducing Pdx1-positive pancreatic progenitor cells to differentiate into NKX6.1-positive pancreatic progenitor cells (e.g., alone, or with any combination of at least one growth factor from the FGF family, at least one retinoic acid signaling pathway activator, ROCK inhibitor, and at least one growth factor from the TGF-β superfamily) can be used in the method provided herein. In some cases, the SHH pathway inhibitor comprises Sant-1.

Any RA signaling pathway activator capable of inducing Pdx1-positive pancreatic progenitor cells to differentiate into NKX6.1-positive pancreatic progenitor cells (e.g., alone, or with any combination of at least one growth factor from the FGF family, at least one SHH pathway inhibitor, ROCK inhibitor, and at least one growth factor from the TGF-β superfamily) can be used. In some cases, the RA signaling pathway activator comprises retinoic acid.

Any ROCK inhibitor capable of inducing Pdx1-positive pancreatic progenitor cells to differentiate into NKX6.1-positive pancreatic progenitor cells (e.g., alone, or with any combination of at least one growth factor from the FGF family, at least one SHH pathway inhibitor, a RA signaling pathway activator, and at least one growth factor from the TGF-β superfamily) can be used. In some cases, the ROCK inhibitor comprises Thiazovivin, Y-27632, Fasudil/HA1077, or 14-1152.

Any activator from the TGF-β superfamily capable of inducing Pdx1-positive pancreatic progenitor cells to differentiate into NKX6.1-positive pancreatic progenitor cells (e.g., alone, or with any combination of at least one growth factor from the FGF family, at least one SHH pathway inhibitor, a RA signaling pathway activator, and ROCK inhibitor) can be used. In some cases, the activator from the TGF-β superfamily comprises Activin A or GDF8.

In some cases, the Pdx1-positive, NKX6.1-positive pancreatic progenitor cells are obtained by contacting Pdx1-positive pancreatic progenitor cells under conditions that promote cell clustering with KGF, Sant1, and RA, for a period of 5 days. In some cases, the Pdx1-positive, NKX6.1-positive pancreatic progenitor cells are obtained by contacting Pdx1-positive pancreatic progenitor cells under conditions that promote cell clustering with KGF, Sant1, RA, Y27632, and Activin A, for a period of 5 days. In some cases, the Pdx1-positive, NKX6.1-positive pancreatic progenitor cells are obtained by contacting Pdx1-positive pancreatic progenitor cells under conditions that promote cell clustering with KGF for a period of 5 days. In some cases, the Pdx1-positive, NKX6.1-positive pancreatic progenitor cells are obtained by contacting Pdx1-positive pancreatic progenitor cells in a S3 medium.

Aspects of the disclosure involve insulin-positive endocrine cells. Insulin-positive endocrine cells of use herein can be derived from any source or generated in accordance with any suitable protocol. In some aspects, NKX6.1-positive pancreatic progenitor cells are differentiated to insulin-positive endocrine cells, In some aspects, the insulin-positive endocrine cells are further differentiated, e.g., by induction or maturation to SC-β cells.

In some aspects, a method of producing an insulin-positive endocrine cell from an NKX6.1-positive pancreatic progenitor cell comprises contacting a population of cells (e.g., under conditions that promote cell clustering) comprising NKX6-1-positive pancreatic progenitor cells with a) a TGF-β signaling pathway inhibitor, and b) a thyroid hormone signaling pathway activator, to induce the differentiation of at least one NKX6.1-positive pancreatic progenitor cell in the population into an insulin-positive endocrine cell, wherein the insulin-positive endocrine ceil expresses insulin. In some cases, insulin-positive endocrine cells express Pdx1, NKX6.1, NKX2.2, Mafb, glis3, Sur 1, Kir6.2, Znt8, SLC2A1, SLC2A3 and/or insulin.

Any TGF-β signaling pathway inhibitor capable of inducing the differentiation of NKX6.1-positive pancreatic progenitor cells to differentiate into insulin-positive endocrine cells (e.g., alone, or in combination with other β cell-maturation factors, e.g., a thyroid hormone signaling pathway activator) can be used. In some cases, the TGF-β signaling pathway comprises TGF-β receptor type I kinase signaling. In some cases, the TGF-β signaling pathway inhibitor comprises Alk5 inhibitor II.

Any thyroid hormone signaling pathway activator capable of inducing the differentiation of NKX6.1-positive pancreatic progenitor cells to differentiate into insulin-positive endocrine cells (e.g., alone, or in combination with other β cell-maturation factors, e.g., a TGF-β signaling pathway inhibitor) can be used. In some cases, the thyroid hormone signaling pathway activator comprises triiodothyronine (T3).

In some cases, the method comprises contacting the population of cells (e.g., NKX6.1-positive pancreatic progenitor cells) with at least one additional factor. In some cases, the method comprises contacting the Pdx1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, and optionally v) a protein kinase inhibitor.

In some cases, the method comprises contacting the population of cells (e.g., NKX6.1-positive pancreatic progenitor cells) with at least one additional factor. In some cases, the method comprises contacting the Pdx1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, and v) at least one bone morphogenic protein (BMP) signaling pathway inhibitor.

Any γ-secretase inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator). In some cases, the γ-secretase inhibitor comprises XXI. In some cases, the γ-secretase inhibitor comprises DAPT.

Any growth factor from the EGF family capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some cases, the at least one growth factor from the EG F family comprises betacellulin. In some cases, at least one growth factor from the EGF family comprises EGF.

Any RA signaling pathway activator capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some cases, the RA signaling pathway activator comprises RA.

Any SHH pathway inhibitor capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used in the method provided herein. In some cases, the SHH pathway inhibitor comprises Sant1.

Any BMP signaling pathway inhibitor capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some cases, the BMP signaling pathway inhibitor comprises LDN193189.

In some cases, the population of cells is optionally contacted with a protein kinase inhibitor. In some cases, the population of cells is not contacted with the protein kinase inhibitor. In some cases, the population of cells is contacted with the protein kinase inhibitor. Any protein kinase inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator). In some cases, the protein kinase inhibitor comprises staurosporine.

In some cases, the method comprises contacting the population of cells (e.g., NKX6.1-positive pancreatic progenitor cells) with XXI, Alk5i, T3, RA, Sant1, and betacellulin for a period of 7 days, to induce the differentiation of at least one NKX6.1-positive pancreatic progenitor cell in the population into an insulin-positive endocrine cell, wherein the insulin-positive endocrine cell expresses insulin. In some cases, the method comprises contacting the population of cells (e.g., NKX6.1-positive pancreatic progenitor cells) with XXI, Alk5i, T3, RA, Sant1, betacellulin, and LDN193189 for a period of 7 days, to induce the differentiation of at least one NKX6.1-positive pancreatic progenitor cell in the population into an insulin-positive endocrine cell, wherein the insulin-positive endocrine ceil expresses insulin.

In some cases, the method comprises culturing the population of cells (e.g., NKX6.1-positive pancreatic progenitor cells) in a BE5 medium, to induce the differentiation of at least one NKX6.1-positive pancreatic progenitor cell in the population into an insulin-positive endocrine cell, wherein the insulin-positive endocrine cell expresses insulin.

Aspects of the disclosure involve generating non-native pancreatic β cells which resemble endogenous mature β cells in form and function, but nevertheless are distinct from native β cells.

In some cases, the insulin-positive endocrine cells generated using the method provided herein can form a cell cluster, alone or together with other types of cells, e.g., precursors thereof, e.g., stem cell, definitive endoderm cells, primitive gut tube cell, Pdx1-positive pancreatic progenitor cells, or NKX6.1-positive pancreatic progenitor cells. In some cases, the cell cluster comprising the insulin-positive endocrine cells can be reaggregated using the method provided herein. The reaggregation of the cell cluster can enrich the insulin-positive endocrine cells. In some cases, the insulin-positive endocrine cells in the cell cluster can be further matured into pancreatic β cells. For example, after reaggregation, the second cell cluster can exhibit in vitro GSIS, resembling native pancreatic islet. For example, after reaggregation, the second cell cluster can comprise non-native pancreatic β cell that exhibits in vitro GSIS.

Stage 6 cells as provided herein may or may not be subject to the dissociation and reaggregation process as described herein. In some cases, the cell population comprising the insulin-positive endocrine cells can be directly induced to mature into Sc-β cells. In some cases, the maturation factors can comprise at least one inhibitor of TGF-β signaling pathway and thyroid hormone signaling pathway activator as described herein. In some cases, Sc-β cells can be obtained by contacting a population of cells comprising insulin-positive endocrine cells with Alk5i and T3. In some cases, the insulin-positive endocrine cells can be matured in a CMRLs medium supplemented with 10% FBS. In other cases, Sc-β cells can be obtained by culturing the population of cells containing the insulin-positive endocrine cells in a MCDB131 medium that can be supplemented by 2% BSA. In some cases, the MCDB131 medium with 2% BSA for maturation of insulin-positive endocrine cells into Sc-β cells can be comprise no small molecule factors as described herein. In some case, the MCDB131 medium with 2% BSA for maturation of insulin-positive endocrine cells into Sc-β cells can comprise no serum (e.g., no FBS).

One aspect of the present disclosure provides a method of cryopreservation. As provided herein, the cell population comprising non-native pancreatic β cells can be stored via cryopreservation. For instances, the cell population comprising non-native β cells, e.g., Stage 6 cells in some cases, can be dissociated into cell suspension, e.g., single cell suspension, and the cell suspension can be cryopreserved, e.g., frozen in a cryopreservation solution. The dissociation of the cells can be conducted by any of the technique provided herein, for example, by enzymatic treatment. The cells can be frozen at a temperature of at highest −20° C., at highest −30° C., at highest −40° C., at highest −50° C., at highest −60° C., at highest −70° C., at highest −80° C., at highest −90° C., at highest −100° C., at highest −110° C., at highest −120° C., at highest −130° C., at highest −140° C., at highest −150° C., at highest −160° C., at highest −170° C., at highest −180° C., at highest −190° C., or at highest −200° C. In some cases, the cells are frozen at a temperature of about −80° C. In some cases, the cells are frozen at a temperature of about −195° C. Any cooling methods can be used for providing the low temperature needed for cryopreservation, such as, but not limited to, electric freezer, solid carbon dioxide, and liquid nitrogen. In some cases, any cryopreservation solution available to one skilled in the art can be used for incubating the cells for storage at low temperature, including both custom made and commercial solutions. For example, a solution containing a cryoprotectant can be used. The cryoprotectant can be an agent that is configured to protect the cell from freezing damage. For instance, a cryoprotectant can be a substance that can lower the glass transition temperature of the cryopreservation solution. Exemplary cryoprotectants that can be used include DMSO (dimethyl sulfoxide), glycols (e.g., ethylene glycol, propylene glycol and glycerol), dextran (e.g., dextran-40), and trehalose. Additional agents can be added in to the cryopreservation solution for other effects. In some cases, commercially available cryopreservation solutions can be used in the method provided herein, for instance, FrostaLife™, pZerve™, Prime-XV©, Gibco Synth-a-Freeze Cryopreservation Medium, STEM-CELLBANKER®, CryoStor® Freezing Media, HypoThermosol® FRS Preservation Media, and CryoDefend® Stem Cells Media.

In some cases, a cell cluster can be cryopreserved before subject to reaggregation using the method provided herein. In some cases, a cell cluster can be dissociated into cell suspension as provided herein and then cryopreserved. After cryopreservation for a certain period of time, the cryopreserved cells can be thawed and cultured for reaggregation using the method as provided herein. Cryopreservation as provided herein can prolong the availability of the pancreatic β cells or their precursors. In some cases, during differentiation of non-native pancreatic β cells from precursors thereof or stem cells, the intermediate cell population can be preserved following the method provided herein until the non-native pancreatic β cells are desired, e.g., for transplanting into a human patient. In some cases, the cells can be cryopreserved for any desired period of time before their further use or further processing of the cells, e.g., reaggregation. For example, the cells can be cryopreserved for at least 1 day, at least 5 days, at least 10 days, at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, at least 3 years, at least 4 years, at least 5 years, or at least 10 years.

Sc-β cells can exhibit a response to at least one glucose challenge. In some cases, the SC-β cells exhibit a response to at least two sequential glucose challenges. In some cases, the SC-β cells exhibit a response to at least three sequential glucose challenges. In some cases, the SC-β cell exhibits a response to multiple (e.g., sequential) glucose challenges that resembles the response of endogenous human islets to multiple glucose challenges, In some cases, the SC-β cells are capable of releasing or secreting insulin in response to two consecutive glucose challenges. In some cases, the SC-β cells are capable of releasing or secreting insulin in response to three consecutive glucose challenges. In some cases, the SC-β cells are capable of releasing or secreting insulin in response to four consecutive glucose challenges. In some cases, the SC-β cells are capable of releasing or secreting insulin in response to five consecutive glucose challenges. In some cases, the SC-β cells release or secrete insulin in response to perpetual consecutive glucose challenges. In some cases, cells can be assayed to determine whether they respond to sequential glucose challenges by determining whether they repeatedly increase intracellular Ca²⁺, as described in the examples herein.

In some cases, a method as provided herein can start with a cell population comprising NKX6.1-positive pancreatic progenitor cells. NKX6.1-positive cells can be differentiated into NKX6.1-positive and C-peptide-positive endocrine cells by contacting the NKX6.1-positive cells with at least one factor from EGF superfamily, e.g., betacellulin. In some cases, NKX6.1-positive and C-peptide-positive endocrine cells can also be referred to as insulin-positive endocrine cells. In some cases, one characteristic of insulin-positive endocrine cells can be expression of chromogranin A. In some cases, the population comprising insulin-endocrine cells can be dissociated and reaggregated into a cell cluster as described above.

In some cases, conditions that promote cell clustering comprise a suspension culture. In some cases, the period of time comprises a period of time sufficient to maximize the number of cells co-expressing C-peptide and Nkx6-1. In some cases, the period of time is at least 5 days. In some cases, the period of time is between 5 days and 7 days. In some cases, the period of time is at least 7 days. In some cases, the suspension culture is replenished every day (e.g., with β cell-maturation factors). In some cases, a period of time of between 5 days and 7 days maximizes the number of cells co-expressing C-peptide and NKX6.1.

In some cases, at least 15% of the NKX6.1-positive pancreatic progenitor cells in the population are induced to differentiate into insulin-positive endocrine cells. In some cases, at least 99% of the NKX6.1-positive pancreatic progenitor cells in the population are induced to differentiate into insulin-positive endocrine cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into Pdx1-positive pancreatic progenitor cells by a process of contacting the primitive gut tube cells with i) retinoic acid signaling pathway activator, ii) at least one factor from the FGF family, iii) a SHH pathway inhibitor, iv) a BMP signaling pathway inhibitor, v) a PKC activator, and optionally vi) a ROCK inhibitor, for a period of 2 days; d) differentiating at least some of the Pdx1-positive pancreatic progenitor cells into Pdx1-positive, NKX6.1-positive pancreatic progenitor cells by a process of contacting the Pdx1-positive pancreatic progenitor cells under conditions that promote cell clustering with i) at least one growth factor from the FGF family, ii) at least one SHH pathway inhibitor, and optionally iii) a RA signaling pathway activator, and optionally iv) ROCK inhibitor and v) at least one factor from TGFβ superfamily, every other day for a period of 5 days, wherein the NKX6.1-positive pancreatic progenitor cells expresses Pdx1 and NKX6.1; e) differentiating at least some of the Pdx1-positive, NKX6.1-positive pancreatic progenitor cells into Pdx1-positive, NKX6.1-positive, insulin-positive endocrine cells by a process of contacting the Pdx1-positive, NKX6.1-positive pancreatic progenitor cells under conditions that promote cell clustering with i) a TGF-β signaling pathway inhibitor, ii) a TH signaling pathway activator, iii) at least one SHH pathway inhibitor, iv) a RA signaling pathway activator, v) a γ-secretase inhibitor, and vi) at least one growth factor from the epidermal growth factor (EGF) family, every other day for a period of between five and seven days, wherein the Pdx1-positive, NKX6.1, insulin-positive endocrine cells express Pdx1, NKX6.1, NKX2.2, Mafb, glis3, Sur 1, Kir6.2, Znt8, SLC2A1, SLC2A3 and/or insulin; and f) differentiating at least some of the Pdx1-positive, NKX6.1-positive, insulin-positive endocrine cells into SC-β cells by a process of contacting the Pdx1-positive, NKX6.1-positive, insulin-positive endocrine cells under conditions that promote cell clustering with i) a transforming growth factor β (TGF-β) signaling pathway inhibitor, ii) a thyroid hormone signaling pathway activator, and optionally iii) a protein kinase inhibitor, every other day for a period of between 7 and 14 days to induce the in vitro maturation of at least some of the Pdx1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into Pdx1-positive pancreatic progenitor cells by a process of contacting the primitive gut tube cells with i)retinoic acid signaling pathway activator and ii) at least one factor from the FGF family for a period of 2 days; d) differentiating at least some of the Pdx1-positive pancreatic progenitor cells into Pdx1-positive, NKX6.1-positive pancreatic progenitor cells by a process of contacting the Pdx1-positive pancreatic progenitor cells under conditions that promote cell clustering with at least one growth factor from the FGF family every other day for a period of 5 days, wherein the NKX6.1-positive pancreatic progenitor cells expresses Pdx1 and NKX6.1; e) differentiating at least some of the Pdx1-positive, NKX6.1-positive pancreatic progenitor cells into Pdx1-positive, NKX6.1-positive, insulin-positive endocrine cells by a process of contacting the Pdx1-positive, NKX6.1-positive pancreatic progenitor cells under conditions that promote cell clustering with i) a TGF-β signaling pathway inhibitor, ii) a TH signaling pathway activator, iii) at least one SHH pathway inhibitor, iv) a RA signaling pathway activator, v) a γ-secretase inhibitor, vi) at least one growth factor from the epidermal growth factor (EGF) family, and vii) BMP signaling pathway inhibitor, every other day for a period of between five and seven days, wherein the Pdx1-positive, NKX6.1, insulin-positive endocrine cells express Pdx1, NKX6.1, NKX2.2, Mafb, glis3, Sur 1, Kir6.2, Znt8, SLC2A1, SLC2A3 and/or insulin; and f) differentiating at least some of the Pdx1-positive, NKX6.1-positive, insulin-positive endocrine cells into SC-β cells by culturing the Pdx1-positive, NKX6.1-positive, insulin-positive endocrine cells in MCBD131 medium that is supplemented with 2% BSA to induce the in vitro maturation of at least some of the Pdx1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into Pdx1-positive, NKX6.1-positive pancreatic progenitor cells under suitable conditions; b) differentiating at least some of the Pdx1-positive, NKX6.1-positive pancreatic progenitor cells into Pdx1-positive, NKX6.1-positive, insulin-positive endocrine cells by a process of contacting the Pdx1-positive, NKX6.1-positive pancreatic progenitor cells under conditions that promote cell clustering with i) a TGF-β signaling pathway inhibitor, ii) a TH signaling pathway activator, iii) at least one SHH pathway inhibitor, iv) a RA signaling pathway activator, v) a γ-secretase inhibitor, vi) at least one growth factor from the epidermal growth factor (EGF) family, and vii) BMP signaling pathway inhibitor, every other day for a period of between five and seven days, wherein the Pdx1-positive, NKX6.1, insulin-positive endocrine cells express Pdx1, NKX6.1, NKX2.2, Mafb, glis3, Surn, Kir6.2, Znt8, SLC2A1, SLC2A3 and/or insulin; and c) differentiating at least some of the Pdx1-positive, NKX6.1-positive, insulin-positive endocrine cells into SC-β cells by culturing the Pdx1-positive, NKX6.1-positive, insulin-positive endocrine cells in MCBD131 medium that is supplemented with 2% BSA to induce the in vitro maturation of at least some of the Pdx1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating a cell cluster containing pancreatic β cells, the method comprising: a) obtaining a cell population comprising NKX6.1-positive pancreatic progenitor cells; b) differentiating at least some of the NKX6.1-positive pancreatic progenitor cells into NKX6.1-positive, insulin-positive (or C-peptide-positive) endocrine cells by a process of contacting the NKX6.1-positive pancreatic progenitor cells with at least one growth factor from the epidermal growth factor (EGF) family, wherein the Pdx1-positive, NKX6.1, insulin-positive endocrine cells express Pdx1, NKX6.1, NKX2.2, Mafb, glis3, Sur 1, Kir6.2, Znt8, SLC2A1, SLC2A3 and/or insulin; and c) differentiating at least some of the Pdx1-positive, NKX6.1-positive, insulin-positive endocrine cells into pancreatic β cells by culturing the Pdx1-positive, NKX6.1-positive, insulin-positive endocrine cells in MCBD131 medium that is supplemented with 2% BSA to induce the in vitro maturation of at least some of the Pdx1-positive, NKX6.1-positive, insulin-positive endocrine cells into pancreatic β cells, wherein the pancreatic β cells exhibit a GSIS response in vitro and/or in vivo. In some cases, the GSIS response resembles the GSIS response of an endogenous mature β cells.

### Methods of Enriching Stem Cell Derived Beta Cells

Provided herein are methods of isolating or enriching for a population of β cells (e.g., stem cell derived β cells) from a heterogeneous population of cells, e.g., a mixed population of cells comprising β cells (e.g., stem cell derived β cells) or precursors thereof from which the β cells (e.g., stem cell derived 3 cells) cells were derived. A population of β cells (e.g., stem cell derived β cells) produced by any of the above-described processes can be enriched, isolated and/or purified by using a cell surface marker (e.g., CD49a, CD29, CD99, CD10, CD59, CD141, CD165, G46-2.6, CD44, CD57) present on the β cells (e.g., stem cell derived β cells), which is not present on the insulin-positive endocrine cell or precursor thereof from which it was derived, enterochromaffin cells (EC cells), and/or α-cells (e.g., stem cell derived α cells). Such cell surface markers are also referred to as an affinity tag which is specific for a β cell (e.g., stem cell derived β cell). In some embodiments, the cell surface marker is an inducible cell surface marker. For example, CD49a can be induced to the surface by certain signals.

In some embodiments, differentiated β cells (e.g., stem cell derived β cells) can be sorted and enriched from other cells, including a cells and endocrine cells. In some embodiments, differentiated 3 cells (e.g., stem cell derived β cells) can be sorted and enriched from other cells, including a cells, EC cells, and immature insulin-positive endocrine cells, by contacting the population of cells with an agent that binds CD49a. In some embodiments, the agent is an antibody or antigen binding fragment thereof that binds CD49a expressed on the surface of differentiated β cells (e.g., stem cell derived β cells).

In some embodiments, the agent is a ligand or other binding agent that specifically binds CD49a that is present on the cell surface of a differentiated β cells (e.g., stem cell derived β cells) In some embodiments, an antibody which binds to CD49a present on the surface of a SC-β cell (e.g. a human SC-β cell) is used as an affinity tag for the enrichment, isolation or purification of chemically induced (e.g. by contacting with at least one β cell maturation factor as described herein)SC-β cells produced by the methods described herein. Such antibodies are known and commercially available.

The skilled artisan will readily appreciate the processes for using antibodies for the enrichment, isolation and/or purification of SC-β cell. For example, in some embodiments, the reagent, such as an antibody, is incubated with a cell population comprising SC-β cells, wherein the cell population has been treated to reduce intercellular and substrate adhesion. The cell population is then washed, centrifuged and resuspended. In some embodiments, if the antibody is not already labeled with a label, the cell suspension is then incubated with a secondary antibody, such as an FACS-conjugated antibody that is capable of binding to the primary antibody. The SC-β cells are then washed, centrifuged, and resuspended in buffer. The SC-β cell suspension is then analyzed and sorted using a fluorescence activated cell sorter (FACS). Antibody-bound, fluorescent reprogrammed cells are collected separately from non-bound, non-fluorescent cells (e.g. immature insulin-producing cells, a cells, and endocrine cells), thereby resulting in the isolation of SC-β cells from other cells present in the cell suspension, e.g. insulin-positive endocrine cells or precursors thereof, or immature, insulin-producing cell (e.g. other differentiated cell types).

In some embodiments, the isolated cell composition that comprises differentiated β cells (e.g., stem cell derived β cells) can be further purified by using an alternate affinity-based method or by additional rounds of sorting using the same or different markers that are specific for differentiated β cells (e.g., stem cell derived β cells). For example, in some embodiments, FACS sorting is used to first isolate a SC-β cell which expresses NKX6-1, either alone or with the expression of C-peptide, or alternatively with a β cell marker disclosed herein from cells that do not express one of those markers (e.g. negative cells) in the cell population. A second FACS sorting, e.g. sorting the positive cells again using FACS to isolate cells that are positive for a different marker than the first sort enriches the cell population for reprogrammed cells. In an alternative embodiment, FACS sorting is used to separate cells by negatively sorting for a marker that is present on most insulin-positive endocrine cells or precursors thereof but is not present on SC-β cells.

In some embodiments, differentiated β cells (e.g., stem cell derived β cells) are fluorescently labeled without the use of an antibody then isolated from non-labeled cells by using a fluorescence activated cell sorter (FACS). In such embodiments, a nucleic acid encoding GFP, YFP or another nucleic acid encoding an expressible fluorescent marker gene, such as the gene encoding luciferase, is used to label reprogrammed cells using the methods described above. For example, in some embodiments, at least one copy of a nucleic acid encoding GFP or a biologically active fragment thereof is introduced into at least one insulin-positive endocrine cell which is first chemically induced into a SC-β cell, where a downstream of a promoter expressed in SC-β cell, such as the insulin promoter, such that the expression of the GFP gene product or biologically active fragment thereof is under control of the insulin promoter.

In addition to the procedures just described, chemically induced SC-β cells may also be isolated by other techniques for cell isolation. Additionally, SC-β cells may also be enriched or isolated by methods of serial subculture in growth conditions which promote the selective survival or selective expansion of the SC-β cell. Such methods are known by persons of ordinary skill in the art, and may include the use of agents such as, for example, insulin, members of the TGF-beta family, including Activin A, TGF-beta1, 2, and 3, bone morphogenic proteins (BMP-2, -3, -4, -5, -6, -7, -11, -12, and -13), fibroblast growth factors-1 and -2, platelet-derived growth factor-AA, and —BB, platelet rich plasma, insulin-like growth factors (IGF-I, II) growth differentiation factor (GDF-5, -6, -7, -8, -10, -11, -15), vascular endothelial cell-derived growth factor (VEGF), Hepatocyte growth factor (HGF), pleiotrophin, endothelin, Epidermal growth factor (EGF), beta-cellulin, among others. Other pharmaceutical compounds can include, for example, nicotinamide, glucagon like peptide-1 (GLP-1) and II, GLP-1 and 2 mimetibody, Exendin-4, retinoic acid, parathyroid hormone.

Using the methods described herein, enriched, isolated and/or purified populations of differentiated β cells (e.g., stem cell derived β cells) can be produced in vitro from insulin-positive endocrine cells or precursors thereof (which were differentiated from pluripotent stem cells by the methods described herein). In some embodiments, preferred enrichment, isolation and/or purification methods relate to the in vitro production of human differentiated β cells (e.g., stem cell derived β cells) from human insulin-positive endocrine cells or precursors thereof, which were differentiated from human pluripotent stem cells, or from human induced pluripotent stem (iPS) cells. In such an embodiment, where SC-β cells are differentiated from insulin-positive endocrine cells, which were previously derived from definitive endoderm cells, which were previously derived from iPS cells, the SC-β cell can be autologous to the subject from whom the cells were obtained to generate the iPS cells.

Using the methods described herein, isolated cell populations of differentiated β cells (e.g., stem cell derived β cells) are enriched in differentiated β cell (e.g., stem cell derived β cell) content by at least about 2- to about 1000-fold as compared to a population of cells before the chemical induction of the insulin-positive endocrine cell or precursor population. In some embodiments, differentiated β cells (e.g., stem cell derived β cells) can be enriched by at least about 5- to about 500-fold as compared to a population before the chemical induction of an insulin-positive endocrine cell or precursor population. In other embodiments, differentiated β cells (e.g., stem cell derived β cells) cells can be enriched from at least about 10- to about 200-fold as compared to a population before the chemical induction of insulin-positive endocrine cell or precursor population. In still other embodiments, differentiated β cells (e.g., stem cell derived β cells) cell can be enriched from at least about 20- to about 100-fold as compared to a population before the chemical induction of insulin-positive endocrine cell or precursor population. In yet other embodiments, differentiated β cells (e.g., stem cell derived β cells) can be enriched from at least about 40- to about 80-fold as compared to a population before the chemical induction of insulin-positive endocrine cell or precursor population. In certain embodiments, differentiated β cells (e.g., stem cell derived β cells) can be enriched from at least about 2- to about 20-fold as compared to a population before the chemical induction of insulin-positive endocrine cell or precursor population.

Provided herein is a method of selecting a target cell (e.g., differentiated β cells (e.g., stem cell derived β cells)) from a population of cells comprising contacting the target cell with a stimulating compound, wherein the contacting induces a selectable marker (e.g., CD49a) of the target cell to localize to a cell surface of the target cell, and selecting the target cell (e.g., differentiated β cells (e.g., stem cell derived β cells)) based on the localization of the selectable marker (e.g., CD49a) at the cell surface. In some embodiments, the selectable marker comprises CD49a. In some embodiments, the selecting the target cell is by cell sorting. In some embodiments, the selecting comprises contacting the selectable marker of the target cell with an antigen binding polypeptide when the selectable marker is localized to the surface of the target cell. In some embodiments, the antigen binding polypeptide comprises an antibody. In some embodiments, the antigen binding polypeptide binds to the CD49a. In some embodiments, the method further comprises treating the population of cells with a compound (e.g., enzyme) that removes the selectable marker from a cell surface of at least one cell of the target cell population. In some embodiments, the population of target cells is treated with the compound prior to the contacting of the target cell with the stimulating compound. In some embodiments, the compound cleaves the selectable marker from the cell surface of the at least one cell. In some embodiments, the target cell is an endocrine cell. In some embodiments, the stimulating compound comprises glucose. In some embodiments, the endocrine cell is a β cell. In some embodiments, the β cell is an SC-β cell. In some embodiments, selecting the target cell separates the target cell from the one or more cells of the population of cells.

### Pharmaceutical Compositions

In some embodiments, the present disclosure provides pharmaceutical compositions that can utilize non-native pancreatic beta cell populations and cell components and products in various methods for treatment of a disease (e.g., diabetes). Certain cases encompass pharmaceutical compositions comprising live cells (e.g., non-native pancreatic beta cells alone or admixed with other cell types). Other cases encompass pharmaceutical compositions comprising non-native pancreatic beta cell components (e.g., cell lysates, soluble cell fractions, conditioned medium, ECM, or components of any of the foregoing) or products (e.g., trophic and other biological factors produced by non-native pancreatic beta cells or through genetic modification, conditioned medium from non-native pancreatic beta cell culture). In either case, the pharmaceutical composition may further comprise other active agents, such as anti-inflammatory agents, exogenous small molecule agonists, exogenous small molecule antagonists, anti-apoptotic agents, antioxidants, and/or growth factors known to a person having skill in the art.

Pharmaceutical compositions of the present disclosure can comprise non-native pancreatic beta cell, or components or products thereof, formulated with a pharmaceutically acceptable carrier (e.g. a medium or an excipient). The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are suitable for use in contact with the tissues of human beings and animals without excessive toxicity, irritation, allergic response, or other complication. Suitable pharmaceutically acceptable carriers can include water, salt solution (such as Ringer's solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical compositions comprising cellular components or products, but not live cells, can be formulated as liquids. Pharmaceutical compositions comprising living non-native pancreatic beta cells can be formulated as liquids, semisolids (e.g., gels, gel capsules, or liposomes) or solids (e.g., matrices, scaffolds and the like).

Pharmaceutical compositions may comprise auxiliary components as would be familiar to a person having skill in the art. For example, they may contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, beta-mercaptoethylamine, glutathione and similar species, although other anti-oxidant agents suitable for renal administration, e.g. ascorbic acid and its salts or sulfite or sodium metabisulfite may also be employed.

A buffering agent may be used to maintain the pH of formulations in the range of about 4.0 to about 8.0; so as to minimize irritation in the target tissue. For direct intraperitoneal injection, formulations should be at pH 7.2 to 7.5, preferably at pH 7.35-7.45. The compositions may also include tonicity agents suitable for administration to the kidney. Among those suitable is sodium chloride to make formulations approximately isotonic with blood.

In certain cases, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Pharmaceutical compositions comprising cells, cell components or cell products may be delivered to the kidney of a patient in one or more of several methods of delivery known in the art. In some cases, the compositions are delivered to the kidney (e.g., on the renal capsule and/or underneath the renal capsule). In another embodiment, the compositions may be delivered to various locations within the kidney via periodic intraperitoneal or intrarenal injection. Alternatively, the compositions may be applied in other dosage forms known to those skilled in the art, such as pre-formed or in situ-formed gels or liposomes.

Pharmaceutical compositions comprising live cells in a semi-solid or solid carrier are may be formulated for surgical implantation on or beneath the renal capsule. It should be appreciated that liquid compositions also may be administered by surgical procedures. In particular cases, semi-solid or solid pharmaceutical compositions may comprise semi-permeable gels, lattices, cellular scaffolds and the like, which may be non-biodegradable or biodegradable. For example, in certain cases, it may be desirable or appropriate to sequester the exogenous cells from their surroundings, yet enable the cells to secrete and deliver biological molecules (e.g., insulin) to surrounding cells or the blood stream. In these cases, cells may be formulated as autonomous implants comprising living non-native pancreatic beta cells or cell population comprising non-native pancreatic beta cell surrounded by a non-degradable, selectively permeable barrier that physically separates the transplanted cells from host tissue. Such implants are sometimes referred to as “immunoprotective,” as they have the capacity to prevent immune cells and macromolecules from killing the transplanted cells in the absence of pharmacologically induced immunosuppression.

In other cases, various degradable gels and networks can be used for the pharmaceutical compositions of the present disclosure. For example, degradable materials particularly suitable for sustained release formulations include biocompatible polymers, such as poly(lactic acid), poly (lactic-co-glycolic acid), methylcellulose, hyaluronic acid, collagen, and the like.

In other cases, it may be desirable or appropriate to deliver the cells on or in a biodegradable, preferably bioresorbable or bioabsorbable, scaffold or matrix. These typically three-dimensional biomaterials contain the living cells attached to the scaffold, dispersed within the scaffold, or incorporated in an extracellular matrix entrapped in the scaffold. Once implanted into the target region of the body, these implants become integrated with the host tissue, wherein the transplanted cells gradually become established.

Examples of scaffold or matrix (sometimes referred to collectively as “framework”) material that may be used in the present disclosure include nonwoven mats, porous foams, or self-assembling peptides. Nonwoven mats, for example, may be formed using fibers comprising a synthetic absorbable copolymer of glycolic and lactic acids (PGA/PLA), foams, and/or poly(epsilon-caprolactone)/poly(glycolic acid) (PCL/PGA) copolymer.

In another embodiment, the framework is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another embodiment, cells are seeded onto foam scaffolds that may be composite structures. In many of the abovementioned cases, the framework may be molded into a useful shape. Furthermore, it will be appreciated that non-native pancreatic beta cells may be cultured on pre-formed, non-degradable surgical or implantable devices.

The matrix, scaffold or device may be treated prior to inoculation of cells in order to enhance cell attachment. For example, prior to inoculation, nylon matrices can be treated with 0.1 molar acetic acid and incubated in polylysine, PBS, and/or collagen to coat the nylon. Polystyrene can be similarly treated using sulfuric acid. The external surfaces of a framework may also be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

In one aspect, the present disclosure provided devices comprising a cell cluster comprising at least one pancreatic β cell. A device provided herein can be configured to produce and release insulin when implanted into a subject. A device can comprise a cell cluster comprising at least one pancreatic β cell, e.g., a non-native pancreatic β cell. A cell cluster in the device can exhibit in vitro GSIS. A device can further comprise a semipermeable membrane. The semipermeable membrane can be configured to retain the cell cluster in the device and permit passage of insulin secreted by the cell cluster. In some cases of the device, the cell cluster can be encapsulated by the semipermeable membrane. The encapsulation can be performed by any technique available to one skilled in the art. The semipermeable membrane can also be made of any suitable material as one skilled in the art would appreciate and verify. For example, the semipermeable membrane can be made of polysaccharide or polycation. In some cases, the semipermeable membrane can be made of poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates, biodegradable polyurethanes, albumin, collagen, fibrin, polyamino acids, prolamines, alginate, agarose, agarose with gelatin, dextran, polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, polyethylene oxide, or any combinations thereof. In some cases, the semipermeable membrane comprises alginate. In some cases, the cell cluster is encapsulated in a microcapsule that comprises an alginate core surrounded by the semipermeable membrane. In some cases, the alginate core is modified, for example, to produce a scaffold comprising an alginate core having covalently conjugated oligopeptides with an RGD sequence (arginine, glycine, aspartic acid). In some cases, the alginate core is modified, for example, to produce a covalently reinforced microcapsule having a chemoenzymatically engineered alginate of enhanced stability. In some cases, the alginate core is modified, for example, to produce membrane-mimetic films assembled by in-situ polymerization of acrylate functionalized phospholipids, In some cases, microcapsules are composed of enzymatically modified alginates using epimerases, In some cases, microcapsules comprise covalent links between adjacent layers of the microcapsule membrane. In some embodiment, the microcapsule comprises a subsieve-size capsule comprising alginate coupled with phenol moieties. In some cases, the microcapsule comprises a scaffold comprising alginate-agarose. In some cases, the SC-β cell is modified with PEG before being encapsulated within alginate. In some cases, the isolated populations of cells, e.g., SC-β cells are encapsulated in photoreactive liposomes and alginate. It should be appreciated that the alginate employed in the microcapsules can be replaced with other suitable biomaterials, including, without limitation, polyethylene glycol (PEG), chitosan, polyester hollow fibers, collagen, hyaluronic acid, dextran with ROD, BHD and polyethylene glycol-diacrylate (PEGDA), poly(MPC-co-n-butyl methacrylate-co-4-vinylphenyl boronic acid) (PMBV) and poly(vinyl alcohol) (PVA), agarose, agarose with gelatin, and multilayer cases of these.

### Methods of Treatment

Further provided herein are methods for treating or preventing a disease in a subject. A composition comprising the cell clusters resembling endogenous pancreatic islets can be administered into a subject to restore a degree of pancreatic function in the subject. For example, the cell clusters resembling endogenous pancreatic islets can be transplanted to a subject to treat diabetes.

The methods can comprise transplanting the cell cluster disclosed in the application to a subject, e.g., a subject in need thereof. The terms “transplanting” and “administering” can be used interchangeably and can refer to the placement of cells or cell clusters, any portion of the cells or cell clusters thereof, or any compositions comprising cells, cell clusters or any portion thereof, into a subject, by a method or route which results in at least partial localization of the introduced cells or cell clusters at a desired site. The cells or cell clusters can be implanted directly to the pancreas, or alternatively be administered by any appropriate route which results in delivery to a desired location in the subject where at least a portion of the implanted cells or cell remain viable. The period of viability of the cells or cell clusters after administration to a subject can be as short as a few hours, e.g. twenty-four hours, to a few days, to as long as several years. In some instances, the cells or cell clusters, or any portion of the cells or cell clusters thereof, can also be transadministered at a non-pancreatic location, such as in the liver or subcutaneously, for example, in a capsule (e.g., microcapsule) to maintain the implanted cells or cell clusters at the implant location and avoid migration.

A subject that can be treated by the methods herein can be a human or a non-human animal. In some cases, a subject can be a mammal. Examples of a subject include but are not limited to primates, e.g., a monkey, a chimpanzee, a bamboo, or a human. In some cases, a subject is a human. A subject can be non-primate animals, including, but not limited to, a dog, a cat, a horse, a cow, a pig, a sheep, a goat, a rabbit, and the like. In some cases, a subject receiving the treatment is a subject in need thereof, e.g., a human in need thereof.

As used herein, the term “treating” and “treatment” can refer to administering to a subject an effective amount of a composition (e.g., cell clusters or a portion thereof) so that the subject as a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (e.g., partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis.

The methods and compositions provided herein may be used to treat a subject who has, or has a risk (e.g., an increased risk) of developing a disease. In some cases, the disease is diabetes, including, but not limited to, type I diabetes, type II diabetes, type 1.5 diabetes, prediabetes, cystic fibrosis-related diabetes, surgical diabetes, gestational diabetes, and mitochondrial diabetes The disease may also be a diabetes complication, including heart and blood vessel diseases, diabetic nephropathy, diabetic neuropathy, diabetic retinopathy, foot damages, and hearing damages.

The methods can comprise transplanting the cell cluster to a subject using any means in the art. For example the methods can comprise transplanting the cell cluster via the intraperitoneal space, renal subcapsule, renal capsule, omentum, subcutaneous space, or via pancreatic bed infusion. For example, transplanting can be subcapsular transplanting, intramuscular transplanting, or intraportal transplanting, e.g., intraportal infusion. Immunoprotective encapsulation can be implemented to provide immunoprotection to the cell clusters.

### EXAMPLES

The examples below further illustrate the described embodiments without limiting the scope of this disclosure.

### Example 1. Stage 6 Culture Conditions

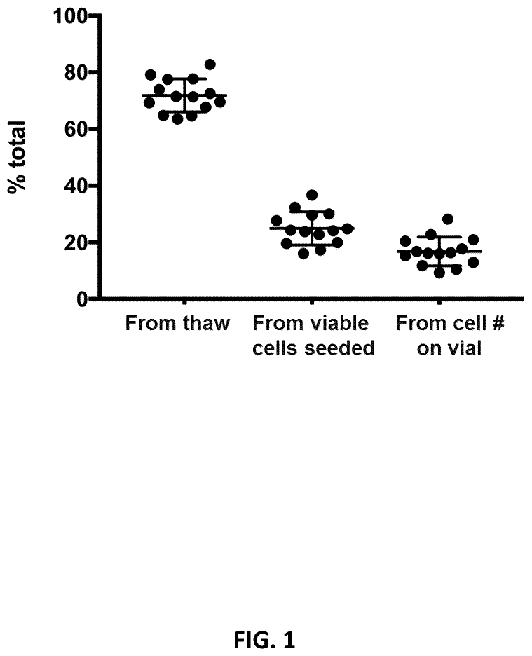

Stage 5 insulin-positive endocrine progenitor cell clusters were differentiated from stem cells as described herein. The insulin-positive endocrine progenitor cell clusters were dissociated such that the majority of the dissociated cells were individual cells, and the cells were cryopreserved, and thawed. The thawed cells are then differentiated into β cell clusters through a stage 6 differentiation. The stage 6 culture medium comprises DMEM F12 base with 1% human serum albumin (HSA), and optionally 1.5 μM zinc (“DS2 medium”). However, the total percentage of cells recovered after stage 6 differentiation using the S6 culture medium was determined to generally be less than 20% of the total number of cells cryopreserved (FIG. 1). Thus, whether alteration of the S6 medium could improve recovery was analyzed.

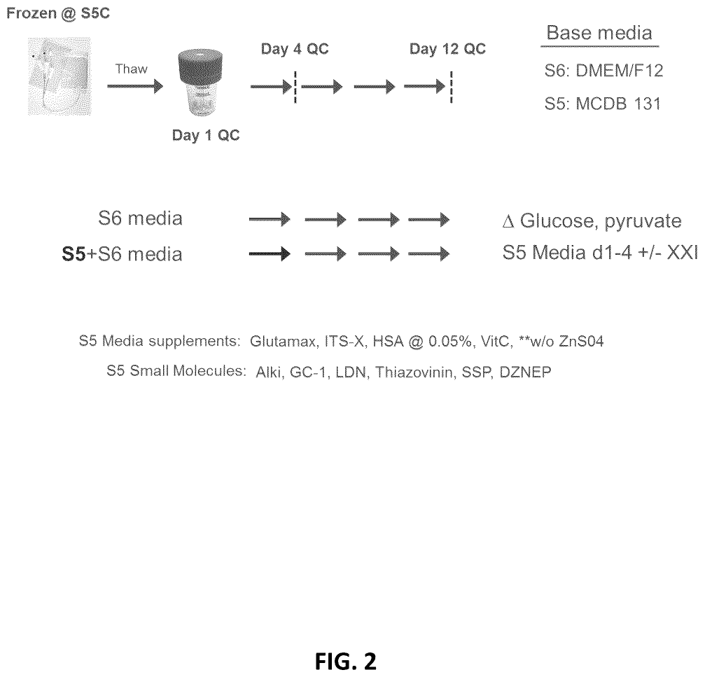

In this instance cryopreserved stage 5 individual cells were thawed and cultured in a modified stage 5 (S5) medium during the first 4 days of Stage 6 (S6d1-d4) (approximately 72 hours), with subsequent culture of the cells in Stage 6 (S6) medium (see experimental outline in FIG. 2).

The original stage 5 culture medium used to differentiate a population of pancreatic progenitor cells into a population of stage 5 insulin-positive endocrine progenitor cell clusters comprises: a base medium of MCDB 131 supplemented with Alk5i (10 μM), GC-1 (1 μM), LDN-193189 (100 nM), thiazovivin (2.5 μM), SSP (3 nM), DZNEP (100 nM), glutamax, ITS-X, low human serum albumin (HSA) (0.05%), vitamin C, XXI, and ZnSO4. The modified stage 5 (S5) medium used during a portion of stage 6 as described herein comprises: a base medium of MCDB 131 supplemented with Alki5i (10 μM), GC-1 (1 μM), LDN-193189 (100 nM), thiazovinin (2.5 μM), SSP (3 nM), DZNEP (100 nM), and low human serum albumin (HSA) (0.05%), and 1 nM zinc (“DS3 medium”).

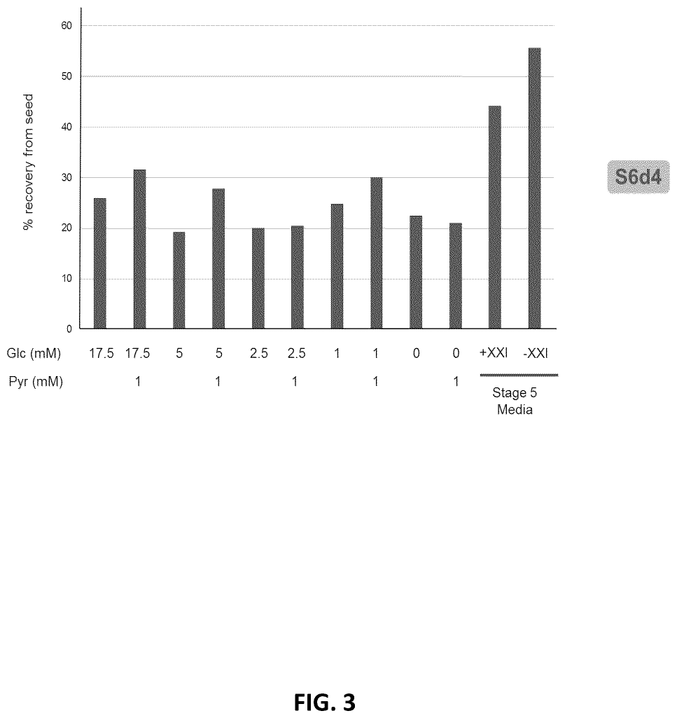

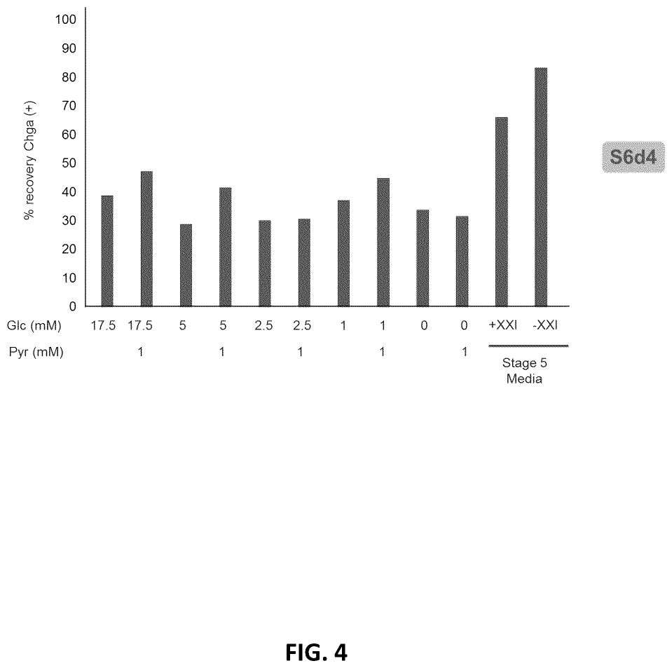

The results indicate that culture of the thawed stage 5 cells in the modified S5 medium for the first 4 days of stage 6 (approximately 72 hours) improves aggregate cell recovery at day 4 (FIG. 3). In addition, the use of the modified S5 resulted in approximately 85% recovery of viable CHGA+ cells at day 4 (approximately 72 hours) (FIG. 4).

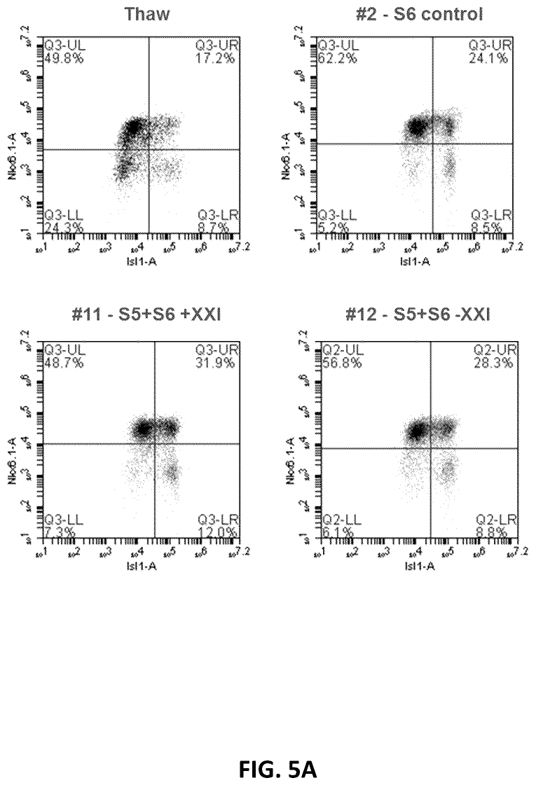

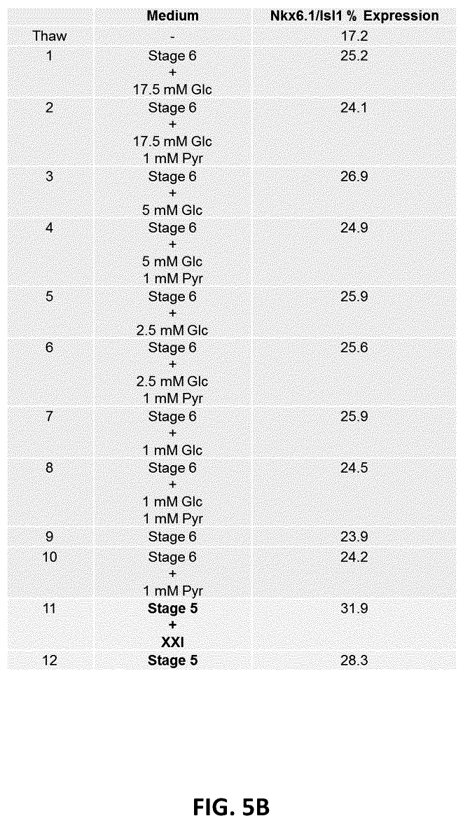

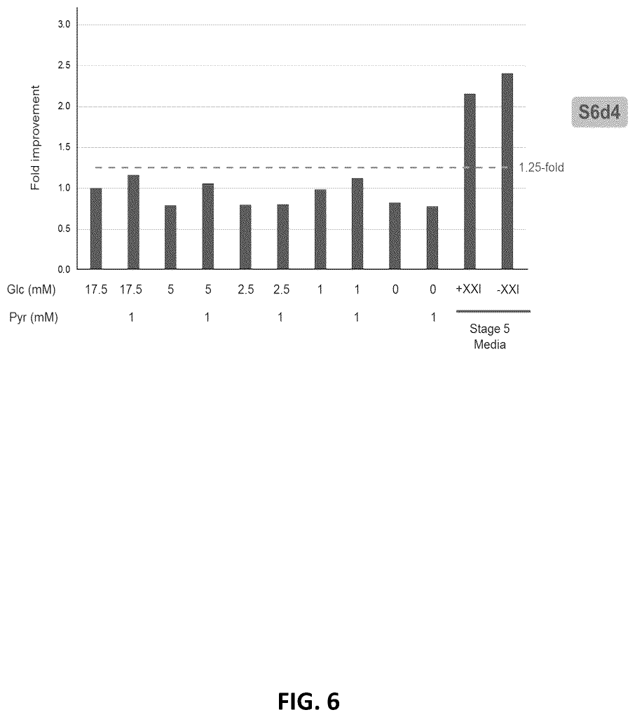

The production of SC-β cells using the modified S5 medium in stage 6 was also analyzed. The results indicate that culturing the thawed stage 5 cells in the modified S5 medium for the first four days stage 6 (approximately 72 hours) results in an increase in the SC-β cell population by day 4 (FIG. 5A bottom FACS plots showing 31.9% (bottom left) and 28.3% (bottom right), Nkx6.1/Isl1 double positive cells, compared to 24.1% Nkx6.1/Isl1 double positive cells with the control S6 medium (top right), see also FIG. 5B). Surprisingly, the use of modified S5 medium to culture the thawed stage 5 cells during the first four days of stage 6 (approximately 72 hours) improved the recovery of SC-β cells approximately 2.5 fold by day 4 of stage 6 (FIG. 6).

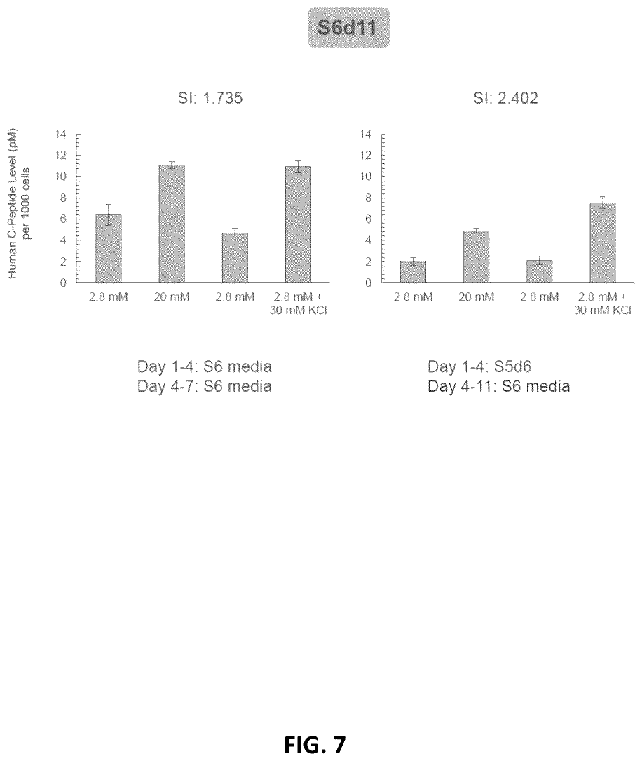

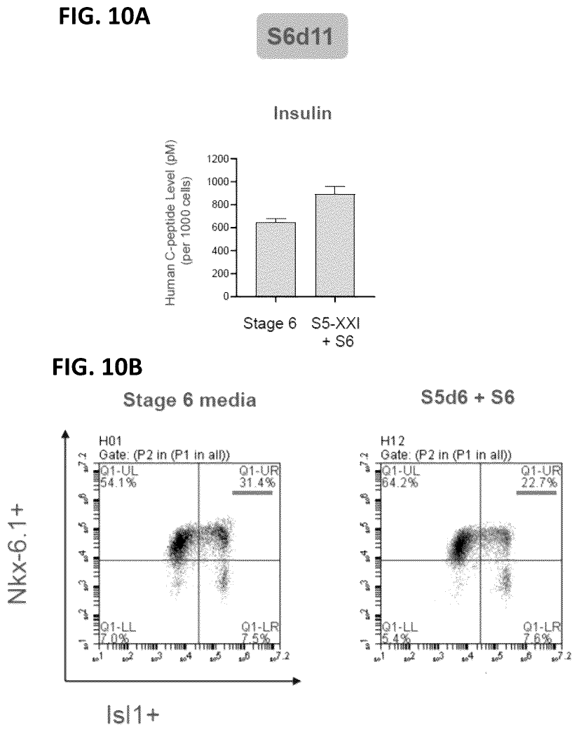

The SC-β cells generated at day 11 of stage 6 (S6d11) using the modified stage S5 medium (days 1-4-approximately 72 hours) and S6 medium (days 4-11) showed GSIS function as well (FIG. 7). There may be a loss in the percent of SC-β cells by day 11 of stage 6 (S6d11) possibly due to the adaptation of the cells from the modified S5 medium to the S6 medium at day 4 (FIG. 10A-B).

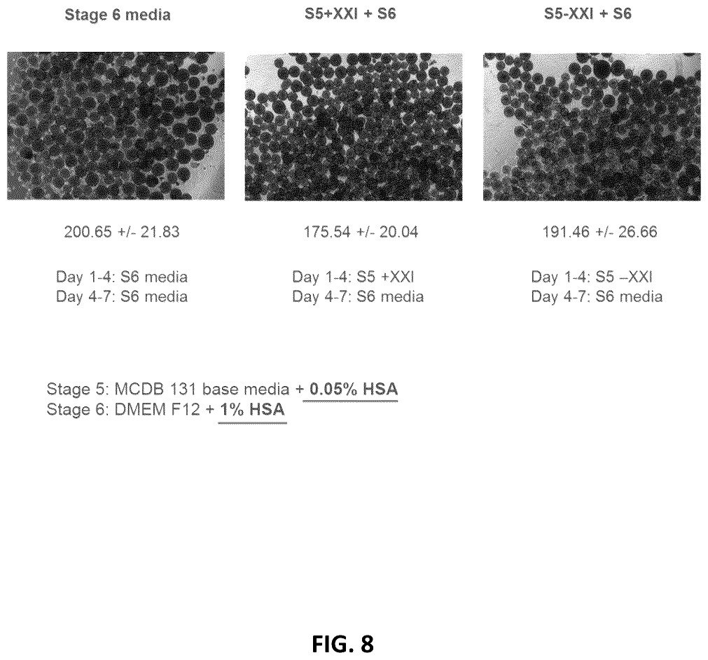

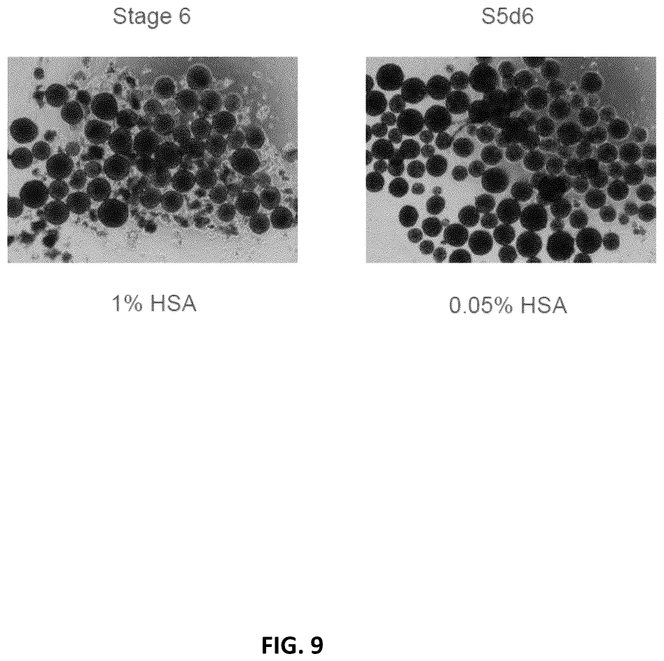

Microscopy analysis also showed that the size and morphology of the cell clusters at day 7 of stage 6 (S6d7) are consistent between cells 1) cultured in the modified S5 medium during days 1-4 of stage 6 (approximately 72 hours) and subsequently cultured in the S6 medium during days 4-7 of stage 6; and 2) cells cultured in the S6 medium during days 1-7 of stage 6 (FIG. 8). Microscopy analysis further shows that the modified stage 5 medium comprising 0.05% HSA enhances the cluster morphology of the stage 6 cells compared to those cultured in only stage 6 medium comprising 1% HSA (FIG. 9).

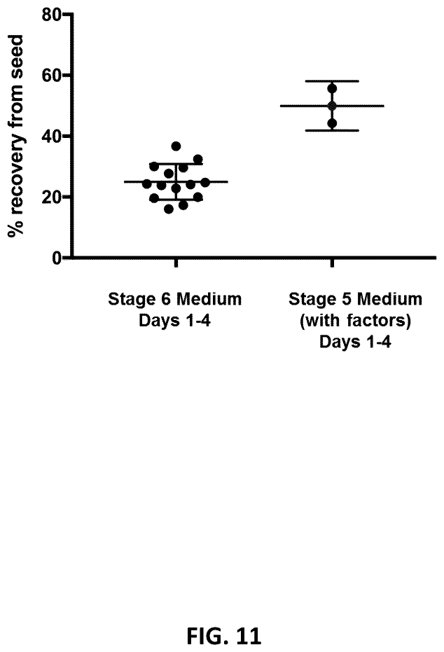

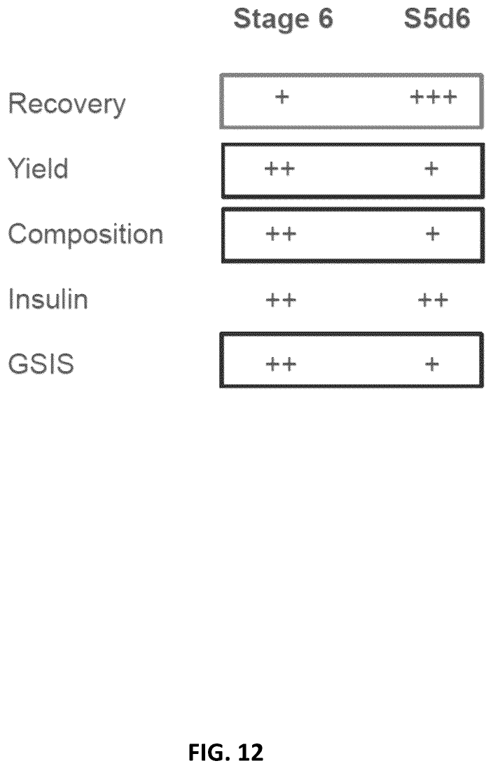

The results overall show an unexpected increase in the percent of cells recovered when the thawed stage 5 cells are cultured in the modified S5 medium during days 1-4 of stage 6 (approximately 72 hours) compared to culturing the thawed stage 5 cells in the S6 medium during days 1-4 of stage 6 (approximately 72 hours), as well as an unexpected enhanced cluster morphology (FIG. 11 and FIG. 12).

### Example 2. Modified Stage 5 (S5) Media for Use in Stage 6

As reported in Example 1, the percent of cells recovered in stage 6 is dramatically increased when the cells are cultured in modified S5 medium for the first 4 days of stage 6 (approximately 72 hours) compared to culturing the cells in S6 medium for the first 4 days (FIG. 11). Therefore, an analysis into the contribution of each factor of the modified S5 medium to the observed increase in cell recovery was analyzed.

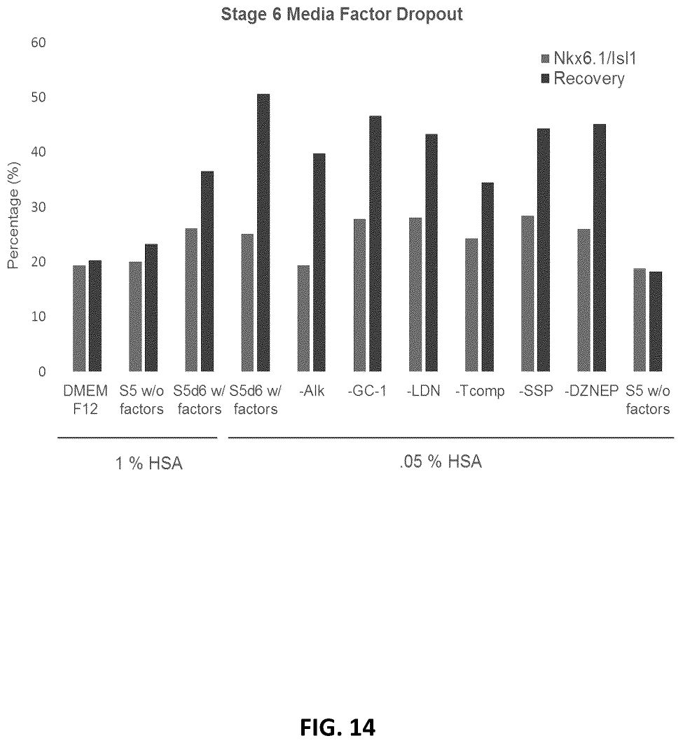

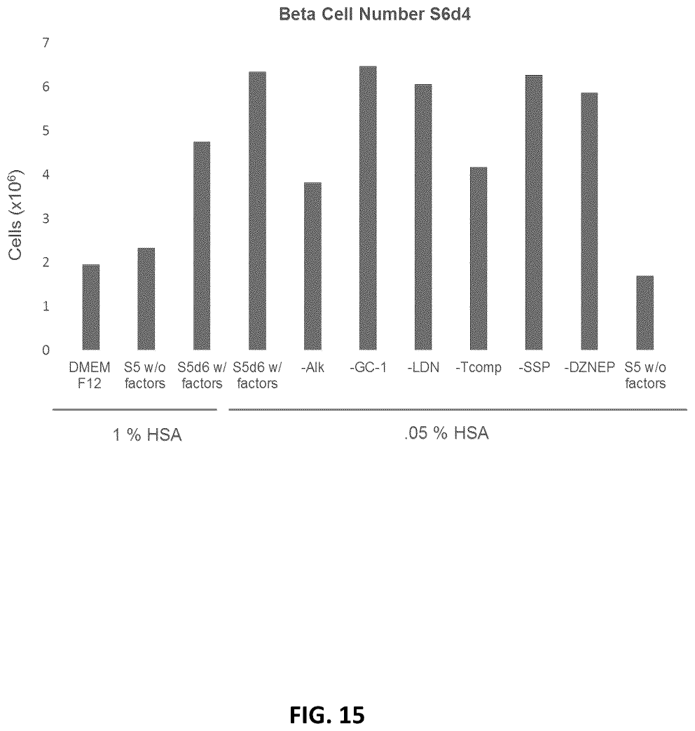

Several factor drop out experiments were conducted to identify the contribution of each factor. Based on this analysis of the individual S5 medium components, low HSA, Alki, and thiazovivin were shown to be key to the improved percentage cell recovery (FIG. 14) and absolute number of cells recovered (FIG. 15), at day 4 of stage 6 (S6d4) observed with the modified S5 medium.

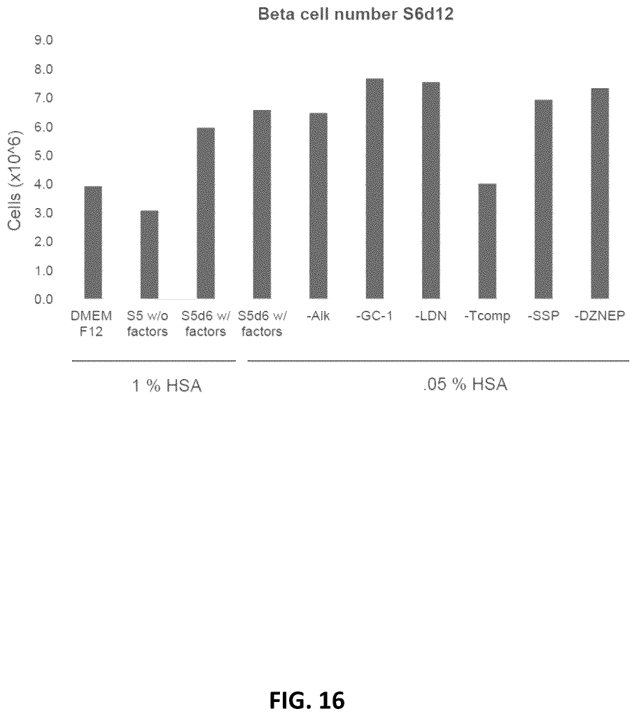

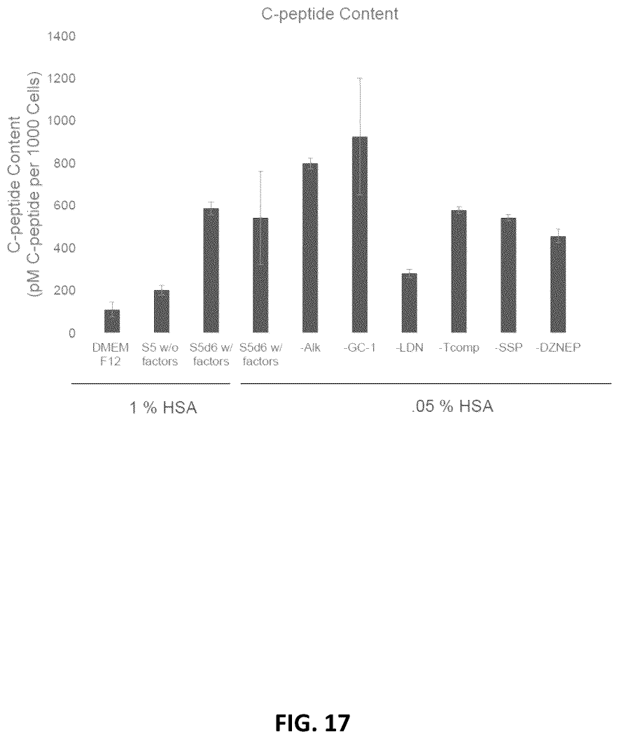

The modified S5 medium was further shown to maintain the number of SC-β cells even after day 4 of stage 6 (S6d4), for example at day 12 (S6d12) (FIG. 16). Likewise, the modified S5 medium was shown to maintain insulin content of the SC-β cells at a level comparable with the use of the S6 medium, as shown by C-peptide content (FIG. 17).

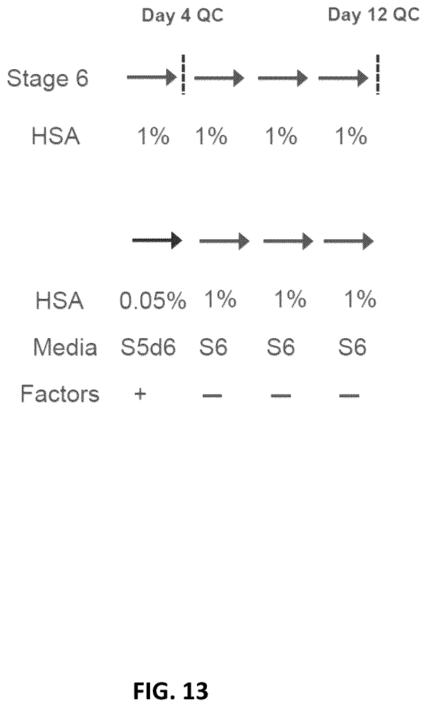

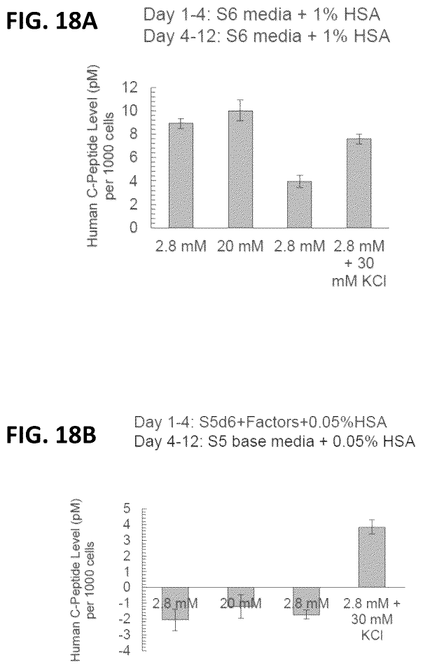

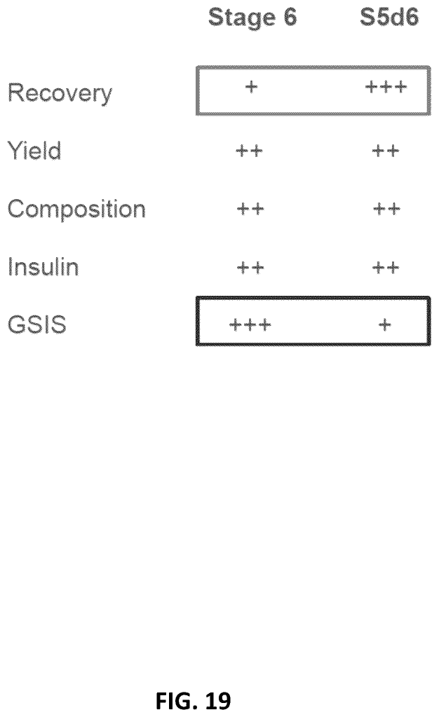

The effect of low HSA in the modified S5 medium was also analyzed. The results indicate that the low HSA in modified S5 media is associated with lower glucose stimulated insulin secretion (GSIS) (FIG. 18A-B). A range of 2.8 mM and 20 mM glucose was used to stimulate GSIS (See experimental protocol FIG. 13).

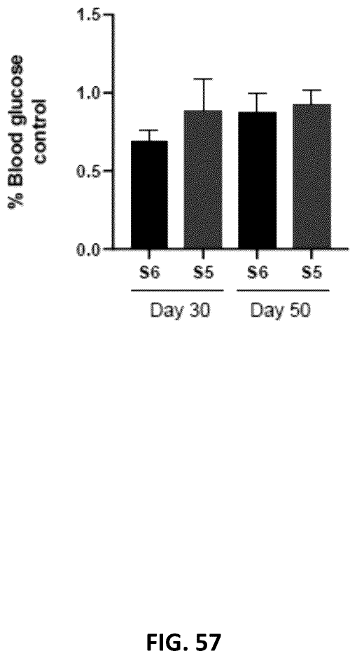

A mouse model was used to examine the in vivo efficacy of SC-islets cultured in the stage 6 medium or the modified stage 5 medium post cryopreservation. The results show that the modified formulation not only improves post-cryopreservation cluster re-aggregation, but the cells also exhibit efficacy in vivo (FIG. 57).

Overall, the results indicate that low HSA, Alki, and thiazovivin are relevant to the improved percentage of cell recovery observed using the modified S5 media; while the low HSA content of the modified S5 medium may correlate with lower GSIS in stage 6 cells. The maintenance of at least some of the stage 5 signaling enhances post-cryopreservation recovery of SC-β cells.

### Example 3. Lipid in Modified Stage 5 (S5) Medium for Use in Stage 6

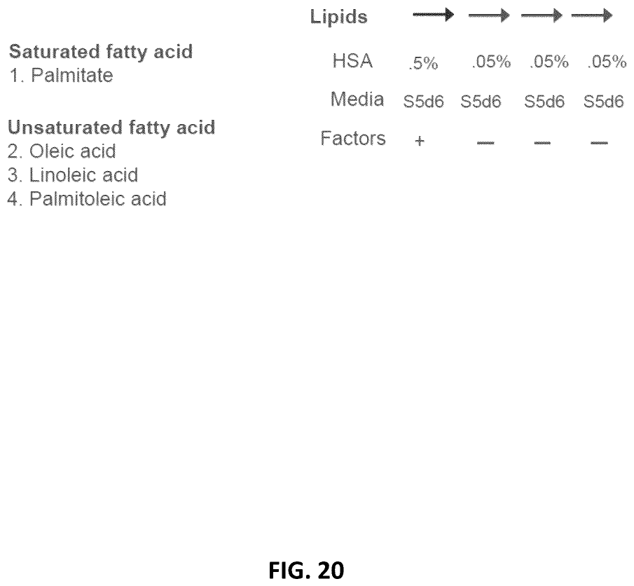

The effect of supplementing the modified S5 medium used in stage 6 with one or more saturated fatty acid of unsaturated fatty acid was analyzed. The fatty acids analyzed included palmitate (saturated fatty acid), oleic acid (unsaturated fatty acid), linoleic acid (unsaturated fatty acid), and palmitoleic acid (unsaturated fatty acid) (see protocol is outlined in FIG. 20). The fatty acid free HSA+fatty acid were stirred for 1 hour at 37° C.

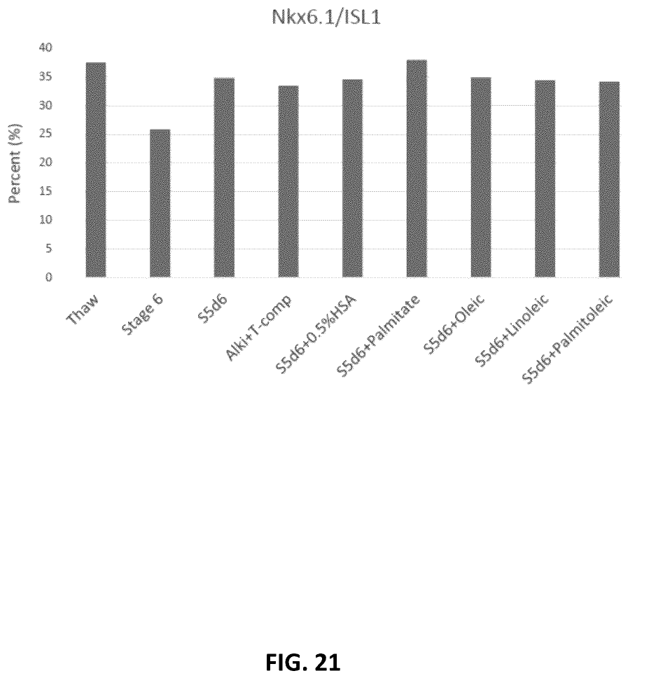

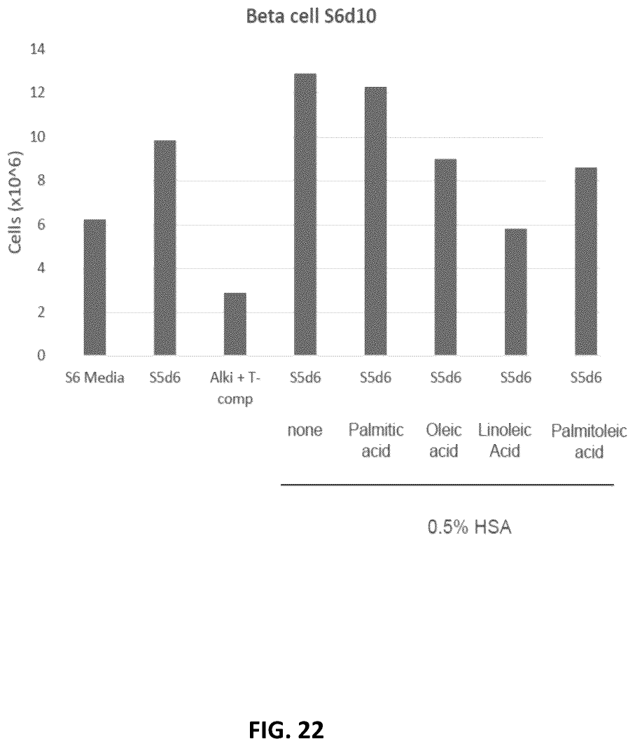

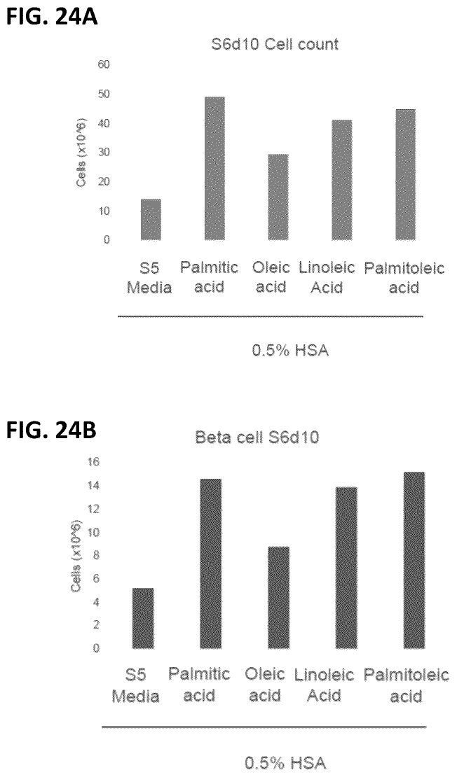

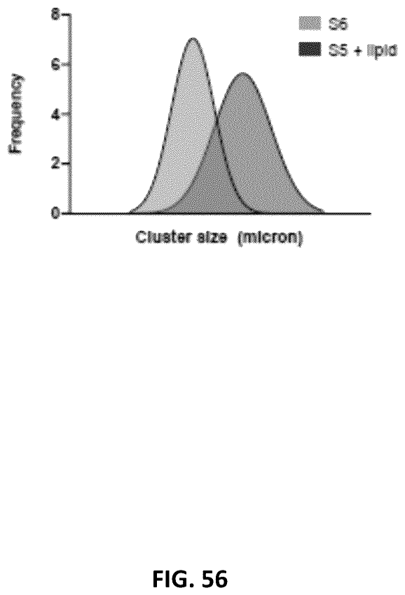

As shown in FIG. 21, the percent of SC-β cells (Nkx6.1/Isl1 double positive cells) recovered was improved with the addition of certain fatty acids to the modified S5 medium, including e.g., palmitate. The effect is further shown in FIG. 22, wherein the addition of palmitic acid and 0.5% HSA enhanced the total number of SC-β cells recovered at day 10 of stage 6. The effect of fatty acid supplementation on the number of cells recovered in stage 6 as well as the number of SC-β cells was also analyzed. The results indicate that supplementation of the modified S5 medium increase both cell yield (FIG. 24A) and SC-β cell numbers (FIG. 24B) at day 10 of stage 6 (S6d10). Lipid supplementation of the modified S5 medium also increases the size of SC-islet clusters (FIG. 56).

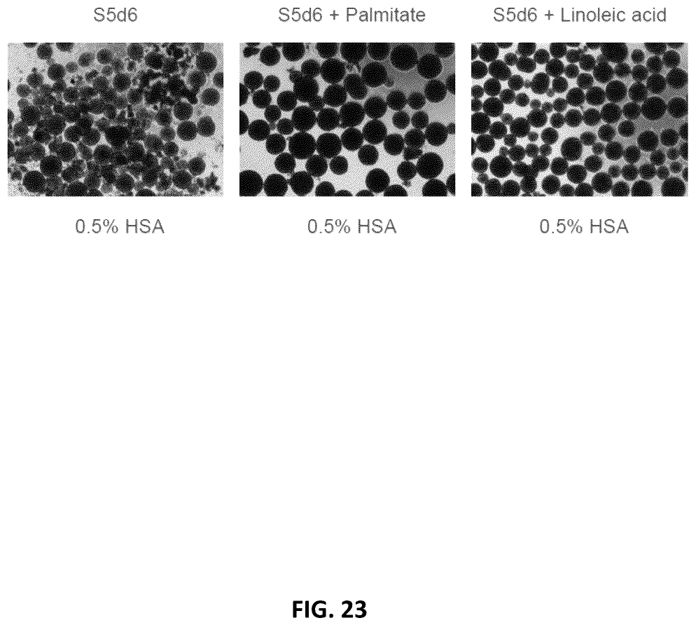

Microscopy analysis showed that the supplementation of fatty acids, including palmitate or linoleic acid enhances the size of the cell clusters at day 6 of stage 6 (S6d6) (FIG. 23).

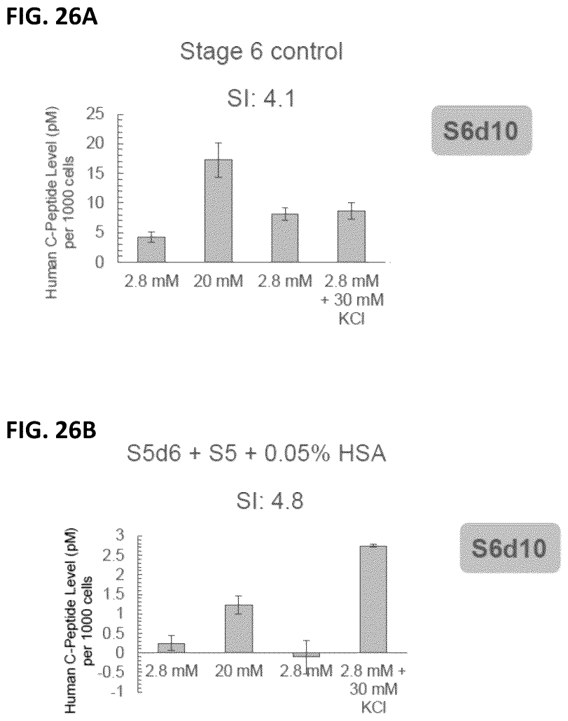

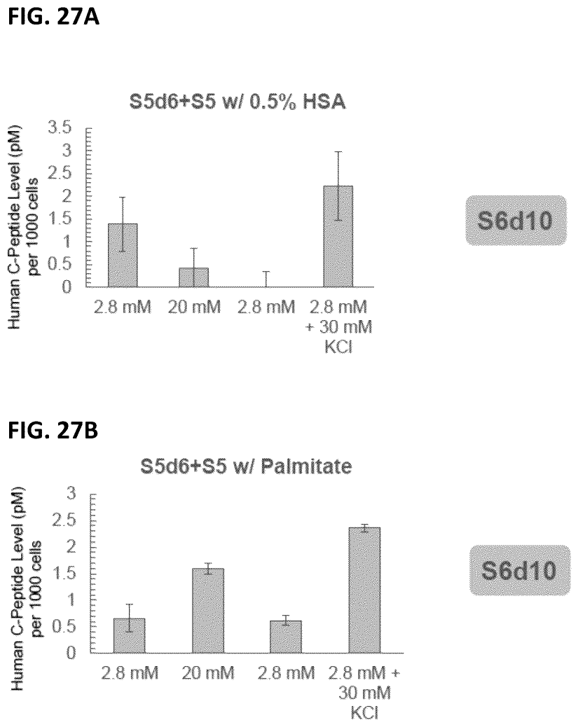

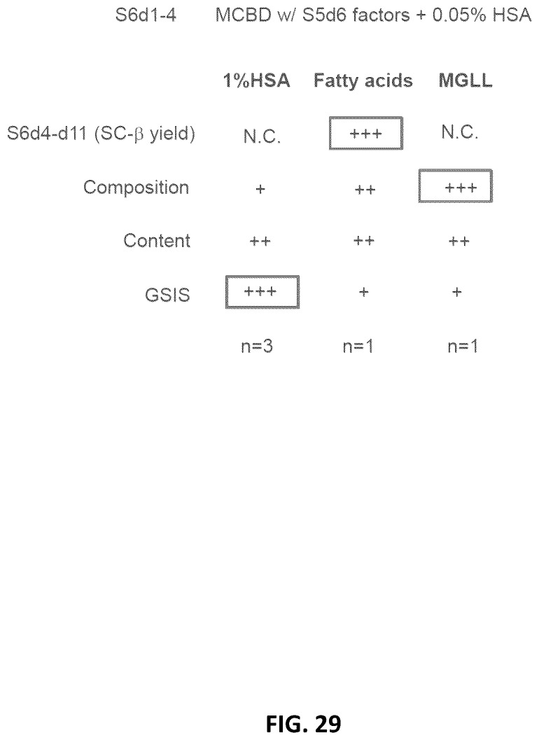

As described in Example 2, the inclusion of the modified S5 medium and low HSA (0.05%) impacts GSIS compared to S6 medium with 1% HSA (FIG. 26A-B). The addition of palmitate post day 4 of stage 6 (S6d4) improves GSIS at day 10 of stage 6 (S6d10) (FIG. 27A-B). The overall results indicate that free fatty acid can increase SC-β cell yield (FIG. 29). The overall results indicate that addition of 1% HSA can increase GSIS function of SC-β cells (FIG. 29). The supplementation of the modified stage 5 media with lipids enhances long term stability of stage 6 SC-islets.

### Example 4. MGLL Inhibitor in Modified Stage 5 (S5) Media for Use in Stage 6

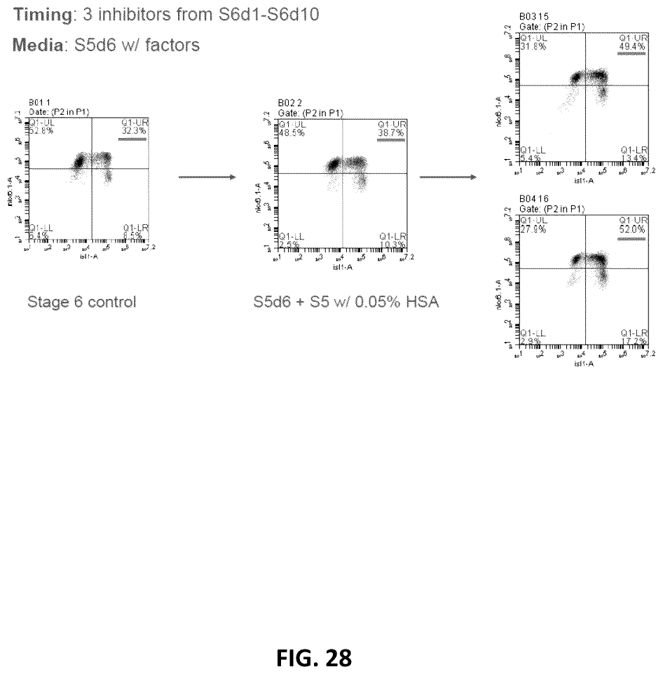

MGLL (monoacylglycerol lipase) is a serine hydrolase that catalyzes the conversion of monoacylglycerides to free fatty acids and glycerol. The effect of three highly specific MGLL inhibitors (JJKK 048 (1 μM), KML 29 (10 μM), NF 1819 (10 μM)) on SC-β cell production was analyzed. The results indicate that when any of the three MGLL inhibitors was added to the culture medium during days 1-10 of stage 6, there is an increase in SC-β cell percent in stage 6 to around 50% (FIG. 28) (Sample 14-JJKK 048 (1 μM); Sample 15-KML 29 (10 μM); Sample 16-NF 1819 (10 μM)). The overall results indicate that MGLL inhibition can increase the percent SC-β cells by selecting against survival of EC-cells (enterochromaffin cells) which are decreased from˜50% to 25% (FIG. 29).

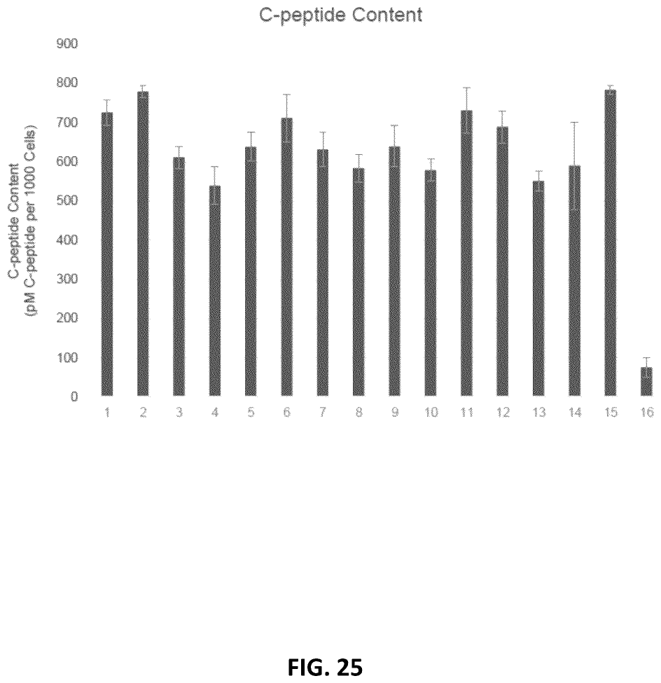

The insulin content across 16 different samples of stage 6 day 14 cells cultured in medium comprising MGLL inhibitors was shown to be consistent (FIG. 25).

### Example 5. Modified Stage 6 Culture Conditions

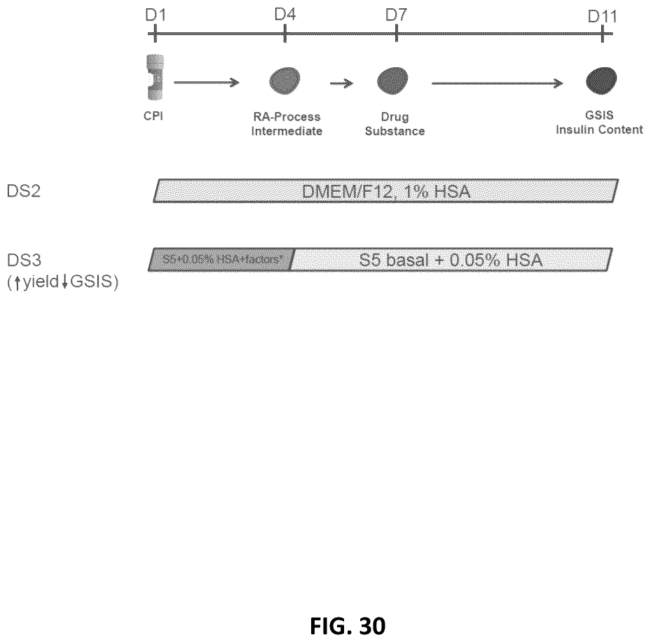

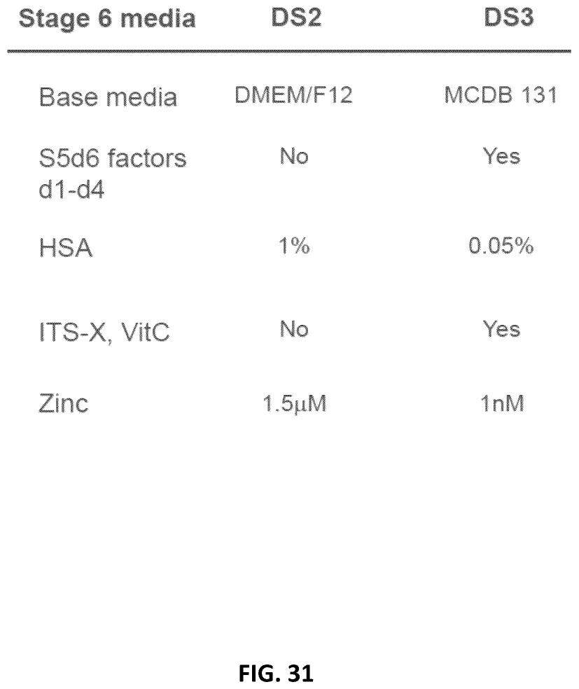

Stage 5 insulin-positive endocrine cell clusters were differentiated from stem cells as described herein. The insulin-positive endocrine cell clusters were dissociated such that the majority of the dissociated cells were now individual cells, and the cells were then cryopreserved, and thawed. The thawed individual cells were then differentiated into β cell clusters through a stage 6 differentiation. The stage 6 culture medium described above comprises MCDB 131 supplemented with S5d6 factors (Alki5i (10 μM), GC-1 (1 μM), LDN-193189 (100 nM), thiazovinin (2.5 μM), SSP (3 nM), DZNEP (100 nM)) throughout days 1-4 of stage 6, along with 0.05% HSA, ITS-X, VitC, and optionally additional agents such as lipids, and MGLL inhibitors (“DS3 medium”). The base medium of MDCB 131 with 0.05% HSA was used for days 5-11 of stage 6 (FIG. 30, FIG. 31).

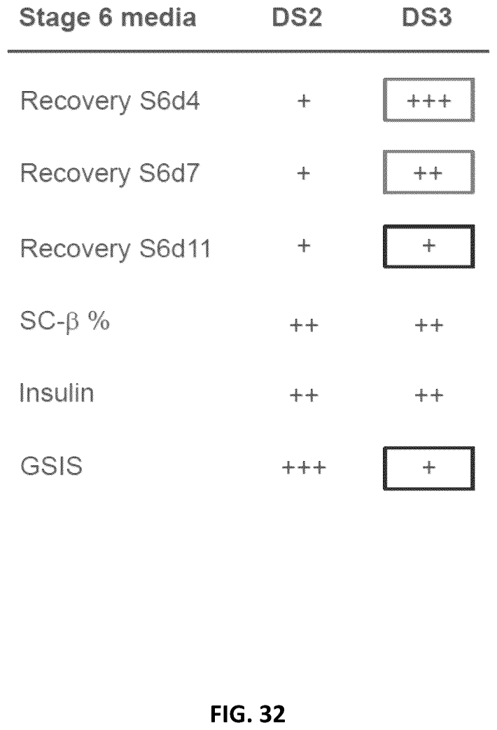

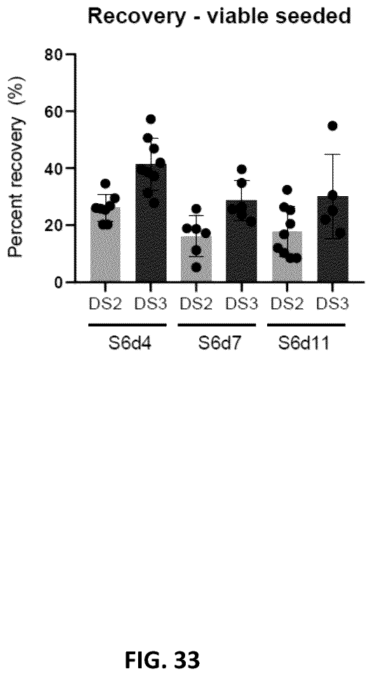

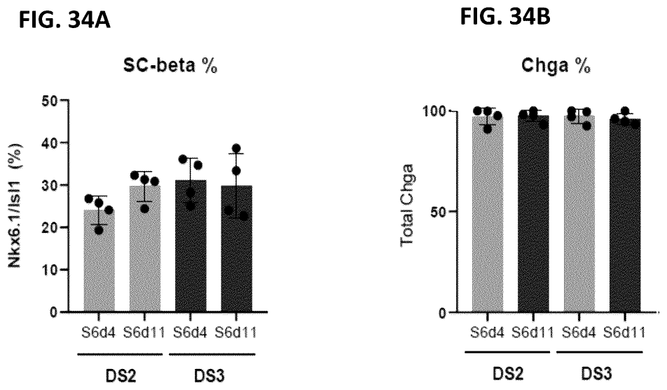

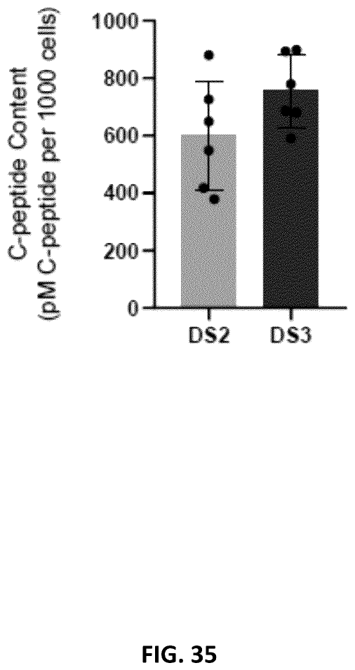

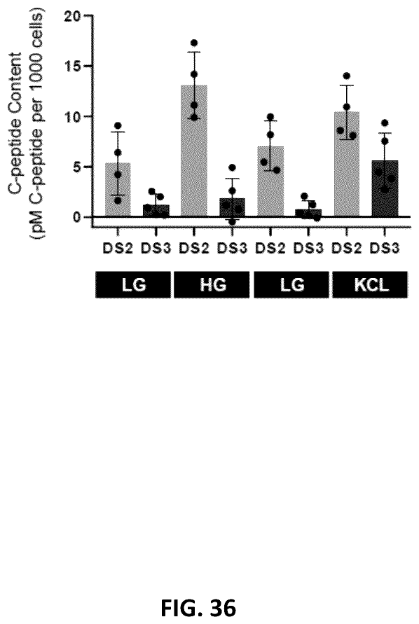

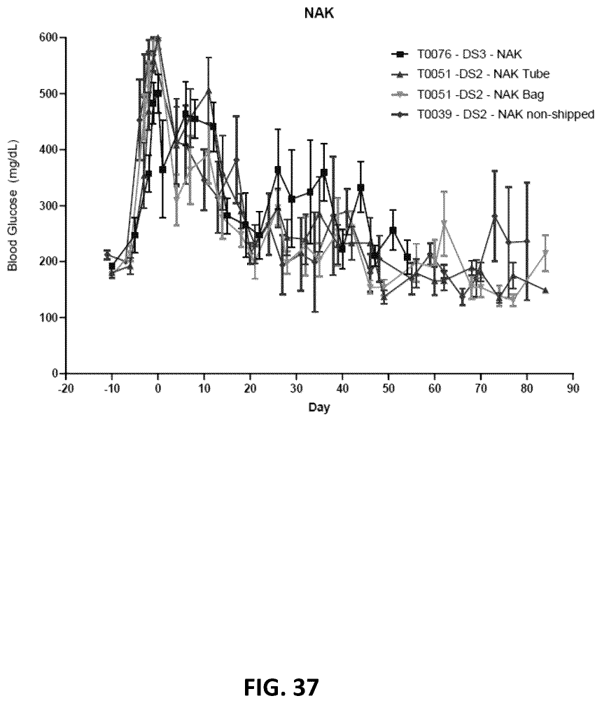

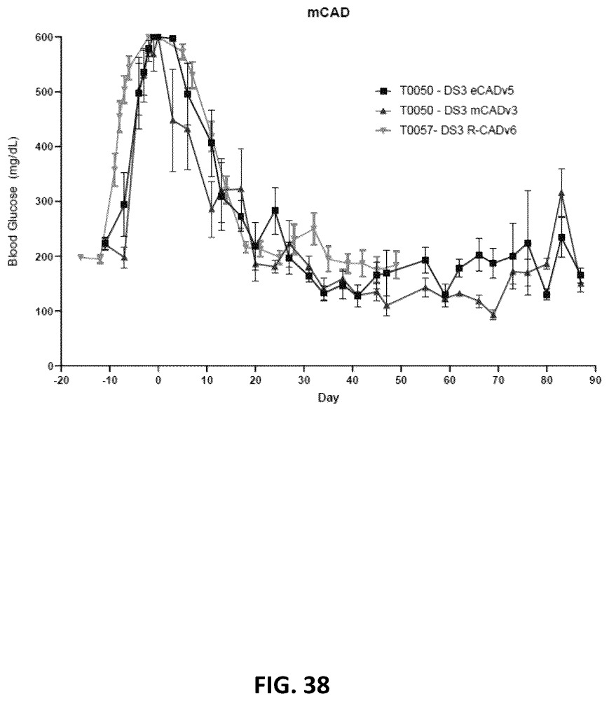

SC-β cells differentiated in DS2 or DS3 medium are capable of functioning in vivo (FIG. 37, FIG. 38). While, the DS3 medium greatly enhanced the recovery of cells at S6d5 and S6d7 (FIG. 33), and retained the percentage of stem cell derived β cells (SC-β cells) (FIG. 34A) and insulin content (FIG. 35) (compared to the original medium DMEM/F12 with 1% HSA (“DS2 medium”)), the DS3 medium did not improve cell recovery at S6d11 and glucose stimulated insulin secretion (GSIS) function in vitro was decreased (FIG. 36) (See summary in FIG. 32). Therefore, a series of experiments were conducted to increase recovery and yield (e.g., maximize re-aggregation efficiency and minimize cell loss over time), improve cell cluster composition (e.g., increase SC-β cell number, and reduce the number of enterochromaffin cell (EC-cells)), and improve function of the cells (e.g., improve SC-β cell GSIS, increase SC-β cell insulin content, and improve glucose control in animals).

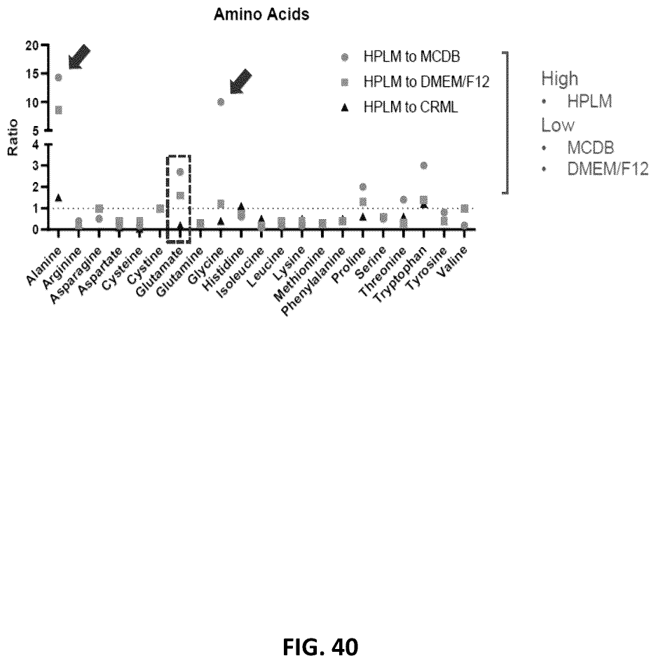

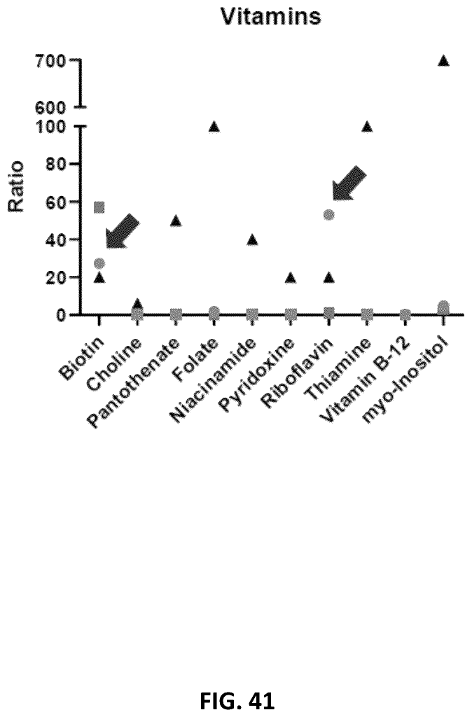

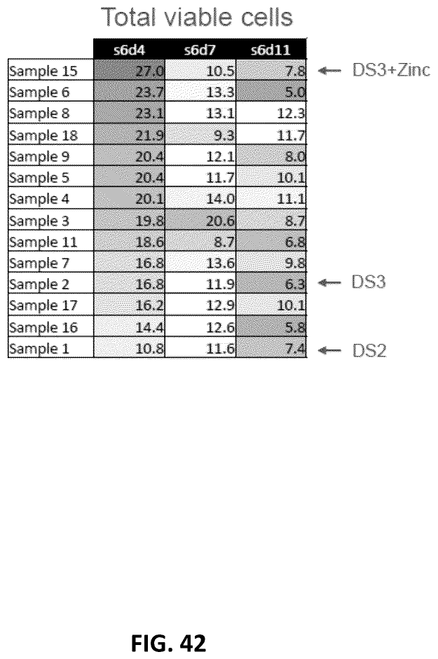

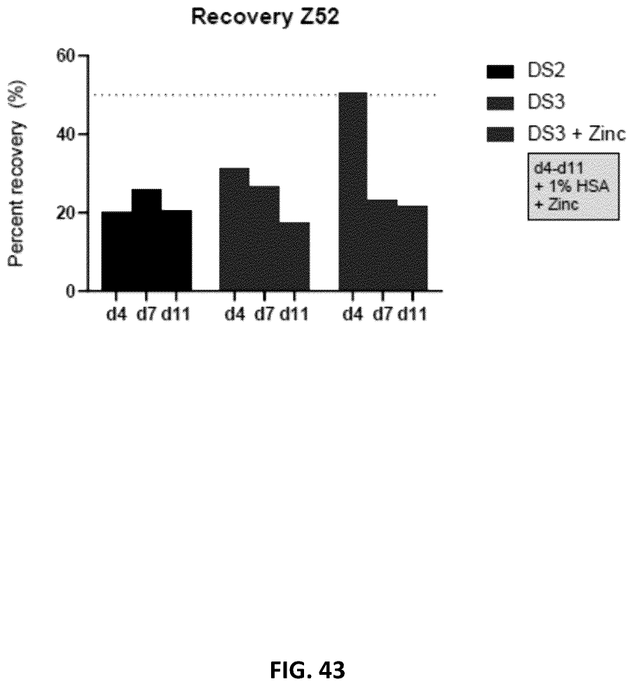

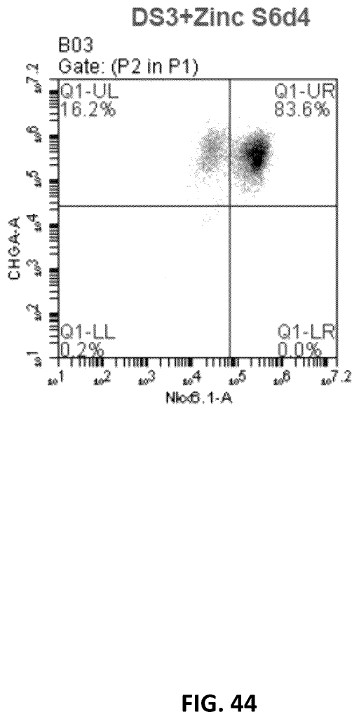

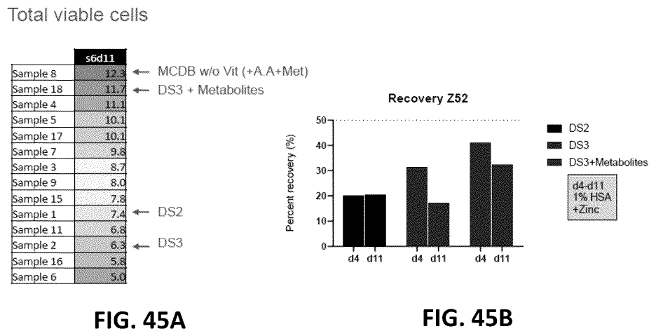

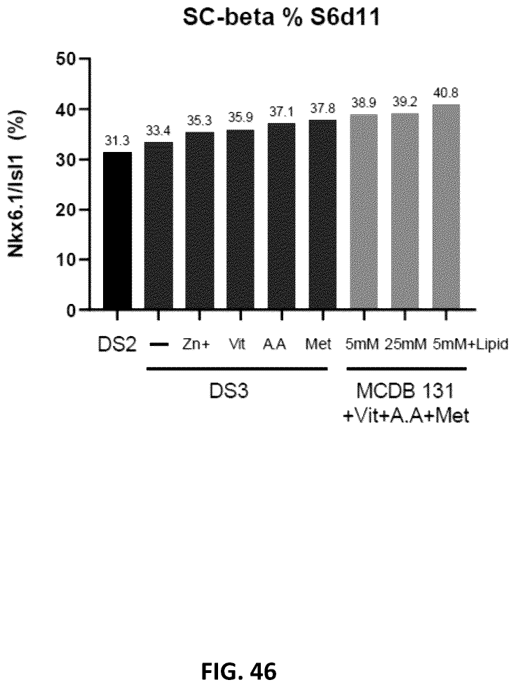

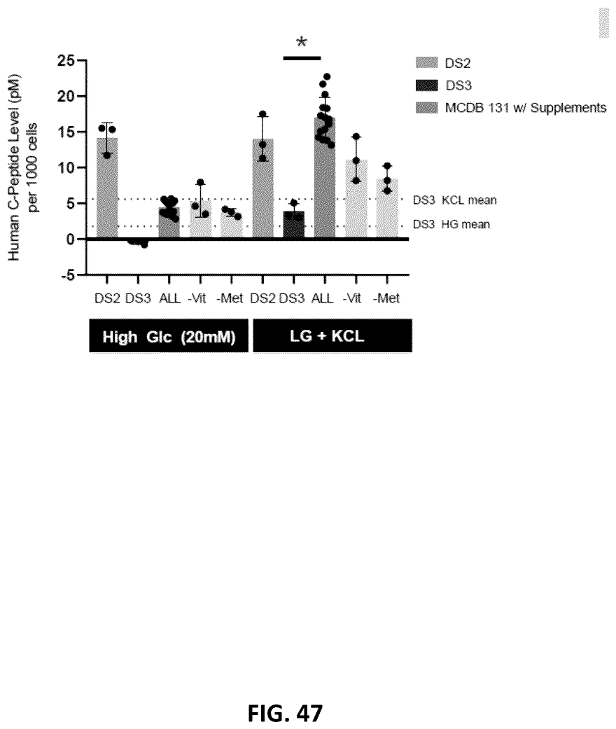

An analysis of the MCDB 131 base medium determined that the MCDB 131 medium contains a lower level of specific amino acids (FIG. 40) and specific vitamins (FIG. 41). As shown in FIG. 45B, inclusion of metabolites and vitamins in DS3 media improved S6d11 recovery (about 30% cell recovery). As shown in FIG. 46, additional supplements such as vitamins, amino acids, metabolites, and lipids to the DS3 medium can improve SC-β cell percentage through S6d11. However, supplementation of DS3 medium with the components vitamins, metabolites, and amino acids was insufficient to improve the high glucose mediated GSIS (FIG. 47). As shown in FIGS. 42, 43, and 44, inclusion of ZnSO4 to the DS3 medium greatly improved S6d4 cell recovery, while there remained a decrease in cell recovery post S6d4.

In summary, the above experiments show that zinc in S6dl-d4 improves recovery of cells from thaw; MCDB 131 supplemented with amino acid, vitamins, and additional metabolites improves cell recovery through S6d11; supplemented MCBD 131 is insufficient to establish high glucose GSIS to DS2 levels; supplemented MCDB generates SC-islets with GSIS (KCL peak) identical to DS2; and MCDB 131 base medium with 1% HSA may be insufficient to stabilize some SC-islets from s6d4-S6d11.

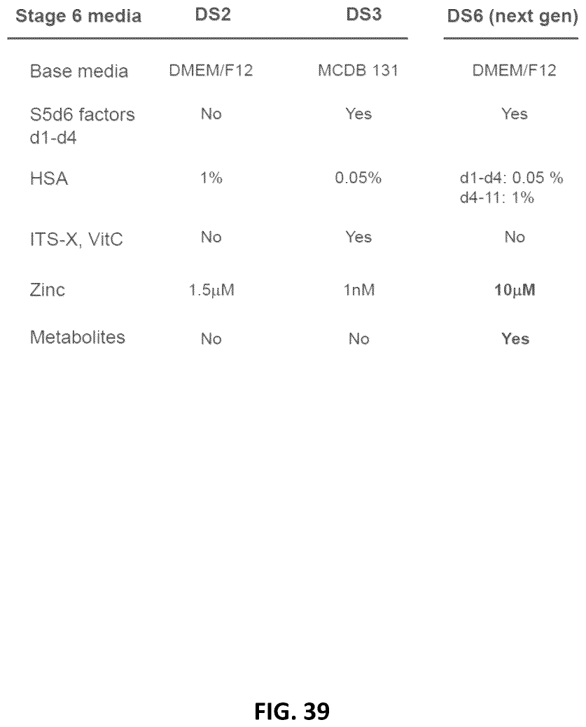

A modified stage 6 medium comprising DMDM/F12 base media, 10 μM zinc, metabolites, S5d6 factors (for days 1-4 of stage 6 only) (Alk5i (10 μM), GC-1 (1 μM), LDN-193189 (100 nM), thiazovinin (2.5 μM), SSP (3 nM), DZNEP (100 nM)), and HSA (0.05% for days 1-4, and 1% for days 5-11) (“DS6 medium”) was made (FIG. 39). A select number of metabolites were included in the DS6 medium, including: glutamate, acetate, β hydroxybutarate, L-caratine, taurine, formate, and biotin.

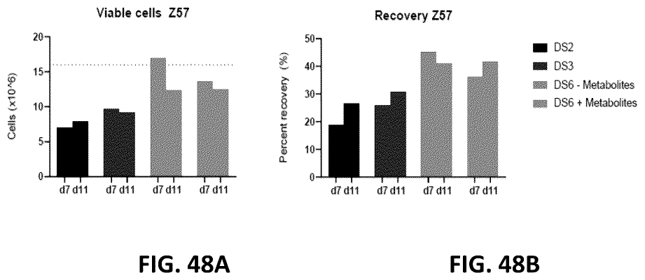

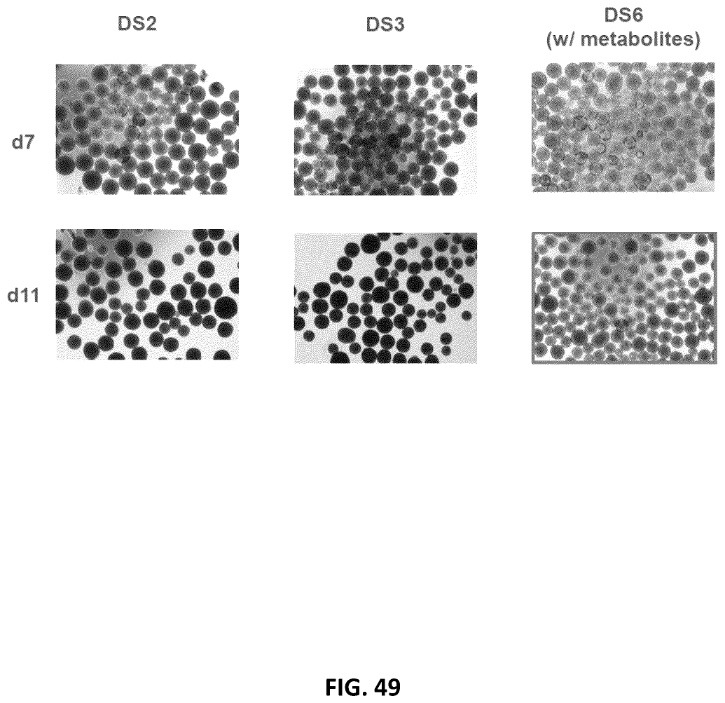

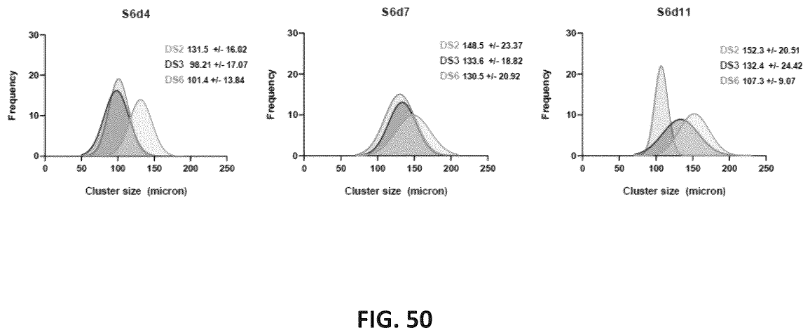

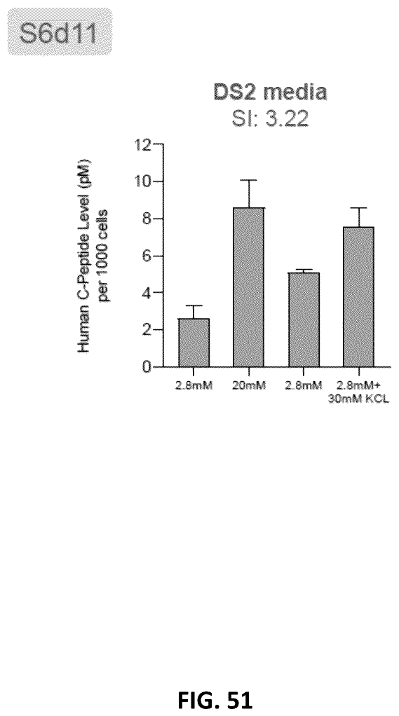

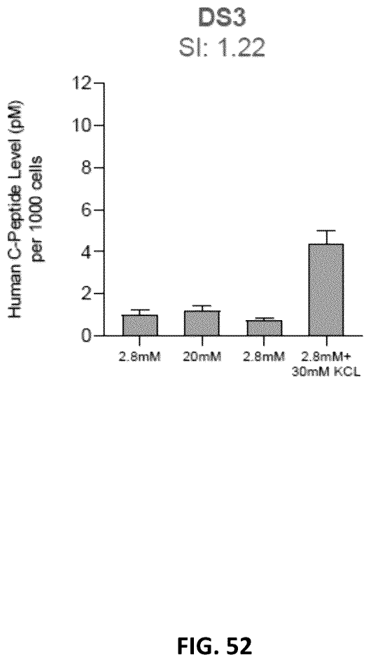

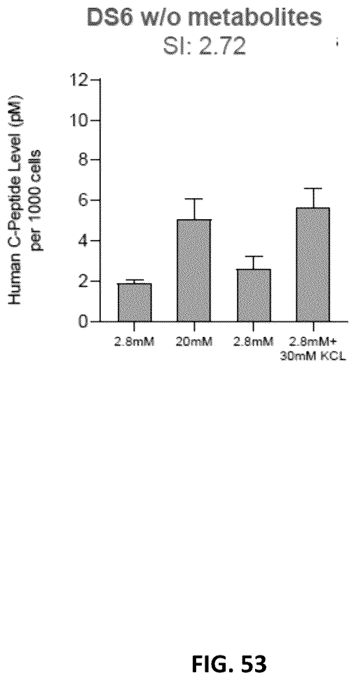

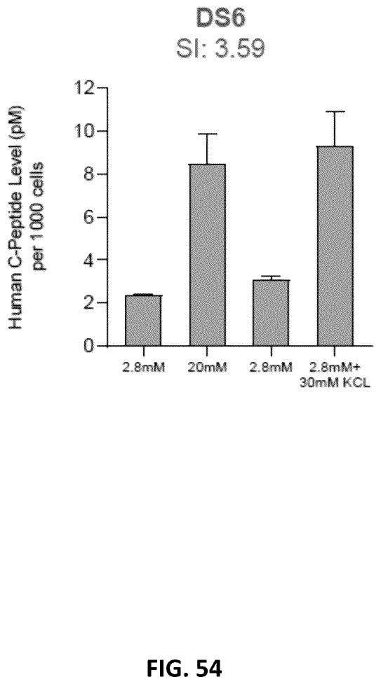

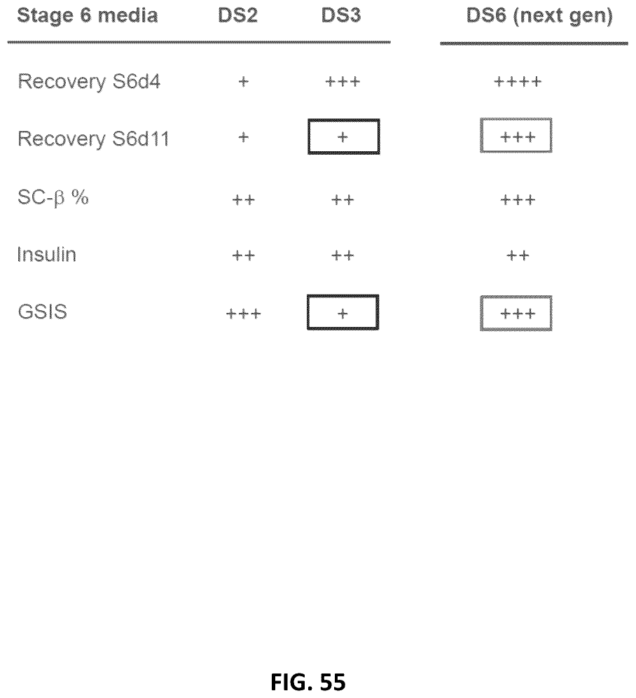

As shown in FIG. 48B, the DS6 medium improves stage 6 cells recovery (40% recovery at S6d11). Furthermore, DS6 cell clusters are smaller (110 microns by S6d11) exhibit more homogeneity through stage 6 as compared to DS2 or DS3 medium (FIG. 49, FIG. 50). SC-islets differentiated in DS3 media do not exhibit GSIS as is observed in SC-islets differentiated in DS2 media (FIG. 51, FIG. 52); while SC-islets cultured in DS6 media without metabolites exhibit improved GSIS at S6d11 (FIG. 53, FIG. 51). However, SC-islets cultured in DS6 media with metabolites exhibit GSIS identical to SC-islets cultured in DS2 media (FIG. 54, FIG. 51). The results indicate that the use of DS6 media in stage 6 results in smaller and more homogenous SC-islets, higher cell recovery, higher cell yield through S6d11, and a better GSIS profile throughout stage 6 (FIG. 55).

What is claimed is:

1. An in vitro composition comprising a plurality of insulin-positive cells and one or more of palmitoleic acid, palmitic acid, oleic acid, and/or linoleic acid.

2. The composition of claim 1, wherein the composition comprises two or more of palmitoleic acid, palmitic acid, oleic acid, and/or linoleic acid.

3. The composition of claim 2, wherein the composition comprises three or more of palmitoleic acid, palmitic acid, oleic acid, and/or linoleic acid.

4. The composition of claim 2, wherein the composition comprises palmitoleic acid, palmitic acid, oleic acid, and/or linoleic acid.

5. The composition of claim 1, wherein the composition comprises a BMP signaling pathway inhibitor.

6. The composition of claim 5, wherein the BMP signaling pathway inhibitor is LDN193189.

7. The composition of claim 1, wherein the composition comprises ZnSO₄.

8. The composition of claim 1, wherein the composition comprises a thyroid hormone signaling pathway activator.

9. The composition of claim 8, wherein the thyroid hormone signaling pathway activator is GC-1 or T3.

10. The composition of claim 1, wherein the composition comprises L-carnitine.

11. The composition of claim 1, wherein the composition comprises palmitoleic acid.

12. The composition of claim 1, wherein the composition comprises palmitic acid.

13. The composition of claim 1, wherein the composition comprises oleic acid.

14. The composition of claim 1, wherein the composition comprises linoleic acid.

15. The composition of claim 1, wherein the plurality of insulin-positive cells are in a plurality of cell clusters.

16. The composition of claim 1, wherein the plurality of insulin-positive cells are dissociated.

17. The composition of claim 1, wherein the composition comprises a plurality of dissociated insulin-positive cells and a plurality of clusters comprise a plurality of insulin-positive cells.

18. The composition of claim 3, wherein the composition comprises ZnSO₄.

19. The composition of claim 18, wherein the composition comprises LDN193189.

20. The composition of claim 19, wherein the composition comprises T3 or GC-1.

21. The composition of claim 3, wherein the composition comprises L-carnitine.

22. The composition of claim 4, wherein the composition comprises ZnSO₄.

23. The composition of claim 22, wherein the composition comprises LDN193189.

24. The composition of claim 23, wherein the composition comprises T3 or GC-1.

25. The composition of claim 4, wherein the composition comprises L-carnitine.

26. The composition of claim 19, wherein the composition comprises L-carnitine.

27. The composition of claim 24, wherein the composition comprises L-carnitine.

28. The composition of claim 20, wherein the plurality of insulin-positive cells are in a plurality of cell clusters.

29. The composition of claim 21, wherein the plurality of insulin-positive cells are in a plurality of cell clusters.

30. The composition of claim 24, wherein the plurality of insulin-positive cells are in a plurality of cell clusters.

31. The composition of claim 27, wherein the plurality of insulin-positive cells are in a plurality of cell clusters.
